SEQUENCE LISTING (37 sequences)

SEQ ID NO: 1 - protein, 379 aa, Homo sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEPVAEEPFTFAMELDDLPKERLKELIFQETARFQPGVLEAP

SEQ ID NO: 2 - protein, 360 aa, Homo sapiens [sequence omitted]

SEQ ID NO: 3 - protein, 53 aa, Homo sapiens
NSDSECPLSHDGYCLHDGVCMYIEALDKYACNCVVGYIGERCQYRDLKWWELR

SEQ ID NO: 4 - DNA, 2599 bp, Homo sapiens
gaaaagactcggggttccgagggccgcagaccgctagccctacgtcacttccgcttcctttcccgcagggcgggtaattcgaacgttttttgcagcgagtggccttcccggttggcgcgcgcccggggcggcggcgctggaggagctcgagacggagcctaagttatgtctgggaggcgaacgcggtccggaggagccgctcagcgctccgggccaagggccccatctcctactaagcctctgcggaggtcccagcggaaatcaggctctgaactcccgagcatcctccctgaaatctggccgaagacacccagtgcggctgcagtcagaaagcccatcgtcttaaagaggatcgtggcccatgctgtagaggtcccagctgtccaatcacctcgcaggagccctaggatttcctttttcttggagaaagaaaacgagccccctggcagggagcttactaaggaggaccttttcaagacacacagcgtccctgccacccccaccagcactcctgtgccgaaccctgaggccgagtccagctccaaggaaggagagctggacgccagagacttggaaatgtctaagaaagtcaggcgttcctacagccggctggagaccctgggctctgcctctacctccaccccaggccgccggtcctgctttggcttcgaggggctgctgggggcagaagacttgtccggagtctcgccagtggtgtgctccaaactcaccgaggtccccagggtttgtgcaaagccctgggccccagacatgactctccctggaatctccccaccacccgagaaacagaaacgtaagaagaagaaaatgccagagatcttgaaaacggagctggatgagtgggctgcggccatgaatgccgagtttgaagctgctgagcagtttgatctcctggttgaatgagatgcagtggggggtgcacctggccagactctccctcctgtcctgtacatagccacctccctgtggagaggacacttagggtcccctcccctggtcttgttacctgtgtgtgtgctggtgctgcgcatgaggactgtctgcctttgagggcttgggcagcagcggcagccatcttggttttaggaaatggggccgcctggcccagccactcactggtgtcctgtctcttgtcgtcctgtccttcctatctccccaaagtaccatagccagtttccagatgggccacagactggggaggagaatcagtggcccagccagaagttaaagggctgagggttgaggtgagaggcacctctgctcttgttgggaggggtggctgcttggaaataggcccaggggctctgccagcctcggcctctccctcctgagttgccttctgttggtggctttcttcttgaacccacctgtgtaaagaggttttcagttccgtgggtttcccctttgattctgtaaatagtcccagagagaattcgtgggctgagggcaattctgtcttggaggaagaagctggacattcagcctgtggagtctgagttttgaaggatgtagggagccttagttgggtctcagaccataagtgtgtactacacagaagctgtgttttctagttctggtctgctgttgagatgtttggtaaatgccaggttgatagggcgctggctgcttggagcaaagggtgcatttcagggtgtggccaccaggtgctgtgagtttctgtggctcatggcctctgggctggtcccttgcacagggcccacgctggagtcttaccactctgctgcaggggtggaaggtggcccctcttgtcacccatacccatttcttacaaaataagttacaccgagtctacttggccctagaagagaaagttgaagagtcccagacctactagcattttgcaactatgcttgtaaagtcctcggaaagtttcctcgcgtaccagacagcggcgggggctgatagcaattttagtttttggcctccctatcctctcacatgagaacactgcctggatgcatctcatgatctctggagaatttccccatctttctcttctttccatcgtgtggattcaatagtgtggatttgaaggctgccctgcccccgactctcctgccgcacccctggccattgtaccttttgatgtttagaagttcgtggaagtagacgctgaggtgtgcagaggagctggtggataacagagaatgccagggaagatgagtgctgggtcagggtacttggatgaaacggtgcaggccaggcgggccctaataaaaccctctgccaggtctgggagtcccaggccatctgctcaacgctctgtggtttgtcagacctgcaagcaagccccctgctggggaagcctaggtgtccttgagctgaaccgcactgaagaactcttgtcctcactggctgatgcagcagaactcttgggaaatgtcttagtcctgcagaatcaggagtcaccagatgatgcagagttgagatcatcattgcaaagttctctgttcctgaggaactaaatttaaggaaaaaatgggattttgttttagagttggaaaaaaagcctgattaaagagtttctgcctgttaaaaaaaaaaaaaaaaaaaaaaa

SEQ ID NO: 5 - protein, 252 aa, Homo sapiens
MSGRRTRSGGAAQRSGPRAPSPTKPLRRSQRKSGSELPSILPEIWPKTPSAAAVRKPIVLKRIVAHAVEVPAVQSPRRSPRISFFLEKENEPPGRELTKEDLFKTHSVPATPTSTPVPNPEAESSSKEGELDARDLEMSKKVRRSYSRLETLGSASTSTPGRRSCFGFEGLLGAEDLSGVSPVVCSKLTEVPRVCAKPWAPDMTLPGISPPPEKQKRKKKKMPEILKTELDEWAAAMNAEFEAAEQFDLLVE

SEQ ID NO: 6 - DNA, 759 bp, Artificial Sequence
  An artificialy synthesized mutant
atgtctgggaggcgaacgcggtccggaggagccgctcagcgctccgggccaagggccccatctcctactaagcctctgcggaggtcccagcggaaatcaggctctgaactcccgagcatcctccctgaaatctggccgaagacacccagtgcggctgcagtcagaaagcccatcgtcttaaagaggatcgtggcccatgctgtagaggtcccagctgtccaatcacctcgcaggagccctaggatttcctttttcttggagaaagaaaacgagccccctggcagggagcttactaaggaggaccttttcaagacacacagcgtccctgccacccccaccagcactcctgtgccgaaccctgaggccgagtccagctccaaggaaggagagctggacgccagagacttggaaatgtctaagaaagtcaggcgttcctacagccggctggagaccctgggctctgcctctacctccaccccaggccgccggtcctgctttggcttcgaggggctgctgggggcagaagacttgtccggagtctcgccagtggtgtgctccaaactcaccgaggtccccagggtttgtgcaaagccctgggccccagacatgactctccctggaatcgccccaccacccgagaaacagaaacgtaagaagaagaaaatgccagagatcttgaaaacggagctggatgagtgggctgcggccatgaatgccgagtttgaagctgctgagcagtttgatctcctggttgaatga

SEQ ID NO: 7 - protein, 252 aa, Artificial Sequence
  An artificialy synthesized mutant
MSGRRTRSGGAAQRSGPRAPSPTKPLRRSQRKSGSELPSILPEIWPKTPSAAAVRKPIVLKRIVAHAVEVPAVQSPRRSPRISFFLEKENEPPGRELTKEDLFKTHSVPATPTSTPVPNPEAESSSKEGELDARDLEMSKKVRRSYSRLETLGSASTSTPGRRSCFGFEGLLGAEDLSGVSPVVCSKLTEVPRVCAKPWAPDMTLPGIAPPPEKQKRKKKKMPEILKTELDEWAAAMNAEFEAAEQFDLLVE

SEQ ID NO: 8 - protein, 9 aa, Artificial
  An artificially synthesized peptide sequence
RKKRRQRRR

SEQ ID NO: 9 - protein, 16 aa, Artificial
  An artificially synthesized peptide sequence
RQIKIWFQNRRMKWKK

SEQ ID NO: 10 - protein, 21 aa, Artificial
  An artificially synthesized peptide sequence
TRSSRAGLQFPVGRVHRLLRK

SEQ ID NO: 11 - protein, 27 aa, Artificial
  An artificially synthesized peptide sequence
GWTLNSAGYLLGKINLKALAALAKKIL

SEQ ID NO: 12 - protein, 18 aa, Artificial
  An artificially synthesized peptide sequence
KLALKLALKALKAALKLA

SEQ ID NO: 13 - protein, 16 aa, Artificial
  An artificially synthesized peptide sequence
AAVALLPAVLLALLAP

SEQ ID NO: 14 - protein, 5 aa, Artificial
  An artificially synthesized peptide sequence
VPMLK

SEQ ID NO: 15 - protein, 5 aa, Artificial
  An artificially synthesized peptide sequence
PMLKE

SEQ ID NO: 16 - protein, 28 aa, Artificial
  An artificially synthesized peptide sequence
MANLGYWLLALFVTMWTDVGLCKKRPKP

SEQ ID NO: 17 - protein, 18 aa, Artificial
  An artificially synthesized peptide sequence
LLIILRRRIRKQAHAHSK

SEQ ID NO: 18 - protein, 21 aa, Artificial
  An artificially synthesized peptide sequence
KETWWETWWTEWSQPKKKRKV

SEQ ID NO: 19 - protein, 18 aa, Artificial
  An artificially synthesized peptide sequence
RGGRLSYSRRRFSTSTGR

SEQ ID NO: 20 - protein, 15 aa, Artificial
  An artificially synthesized peptide sequence
SDLWEMMMVSLACQY

SEQ ID NO: 21 - protein, 12 aa, Artificial
  An artificially synthesized peptide sequence
TSPLNIHNGQKL

SEQ ID NO: 22 - protein, 11 aa, Artificial
  An artificially synthesized peptide sequence
RRRRRRRRRRR

SEQ ID NO: 23 - DNA, 21 bp, Artificial
  An artificially synthesized primer sequence
gaggtgatagcattgctttcg

SEQ ID NO: 24 - DNA, 21 bp, Artificial
  An artificially synthesized primer sequence
caagtcagtgtacaggtaagc

SEQ ID NO: 25 - DNA, 20 bp, Artificial
  An artificially synthesized primer sequence
cgccagagacttggaaatgt

SEQ ID NO: 26 - DNA, 20 bp, Artificial
  An artificially synthesized primer sequence
gtttctgtttctcgggtggt

SEQ ID NO: 27 - DNA, 23 bp, Artificial
  An artificially synthesized primer sequence
gcttgtaaagtcctcggaaagtt

SEQ ID NO: 28 - DNA, 23 bp, Artificial
  An artificially synthesized primer sequence
atctcaactctgcatcatctggt

SEQ ID NO: 29 - RNA, 21 nt, Artificial
  An artificially synthesized origonucleotide sequence for siRNA
nncguacgcggaauacuucga

SEQ ID NO: 30 - RNA, 19 nt, Artificial
  An artificially synthesized origonucleotide sequence for siRNA
ugguuuacaugucgacuaa

SEQ ID NO: 31 - RNA, 19 nt, Artificial
  An artificially synthesized origonucleotide sequence for siRNA
ugguuuacauguuuucuga

SEQ ID NO: 32 - RNA, 19 nt, Artificial
  An artificially synthesized origonucleotide sequence for siRNA
ugguuuacauguuuuccua

SEQ ID NO: 33 - RNA, 19 nt, Artificial
  An artificially synthesized origonucleotide sequence for siRNA
ugguuuacauguuguguga

SEQ ID NO: 34 - RNA, 20 nt, Artificial
  An artificially synthesized origonucleotide sequence for siRNA
gcaguuugaucuccugguuu

SEQ ID NO: 35 - RNA, 21 nt, Artificial
  An artificially synthesized origonucleotide sequence for siRNA
gccagagacuuggaaauguuu

SEQ ID NO: 36 - DNA, 29 bp, Artificial
  CDCA5-F3 primer
ccggaattcatgtctgggaggcgaacgcg

SEQ ID NO: 37 - DNA, 33 bp, Artificial
  CDCA5-R3 primer
ccgctcgagttcaaccaggagatcaaactgctc

(19) United States
(12) Patent Application Publication
(10) Pub. No.: US 2012/0004172 A1
(43) Pub. Date: Jan. 5, 2012

(54) SCREENING METHOD OF ANTI-LUNG OR ESOPHAGEAL CANCER COMPOUNDS

(75) Inventors: Yusuke Nakamura, Tokyo (JP); Yataro Daigo, Tokyo (JP); Takuya Tsunoda, Kanagawa (JP)

(73) Assignee: Oncotherapy Science, Inc., Kanagawa (JP)

(21) Appl. No.: 13/125,549

(22) PCT Filed: Aug. 24, 2009
(86) PCT No.: PCT/JP2009/004054

§ 371 Date: Sep. 13, 2011

Related U.S. Application Data
(60) Provisional application No. 61/197,268, filed on Oct. 27, 2008.

(51) Int. Cl. A61K 38/17; C12N 15/63; A61K 31/7105; C12N 1/21; C12N 1/19; C07K 14/435; C12N 15/11; C12N 5/10

(52) U.S. Cl. 514/9.1; 536/23.1; 435/320.1; 435/358; 435/357; 435/365; 435/348; 435/252.33; 435/252.31; 435/254.2; 530/350; 514/19.3; 514/44 R

(57) ABSTRACT

Disclosed herein is a method for determining a kinase activity of ERK for CDCA5 and methods of screening for modulators of this kinase activity. Also disclosed are methods and pharmaceutical compositions for preventing and/or treating lung cancer or esophageal cancer that use or include such modulators.

### PRIORITY

The present application claims the benefit of U.S. Provisional Application No. 61/197,263, filed Oct. 24, 2008, the entire disclosure of which is hereby incorporated herein by reference.

### TECHNICAL FIELD

The present invention relates to lung and esophageal cancer, more particularly to the diagnosis and treatment thereof.

### BACKGROUND ART

Lung Cancer and Esophageal Cancer

Aerodigestive tract cancer including carcinomas of lung, esophagus, and nasopharynx accounts for nearly one-forth of all cancer deaths in Japan. Lung cancer is the leading cause of cancer-related death in the world, and 1.3 million patients die annually (NPL 1). Two major histologically-distinct types of lung cancer, non-small cell lung cancer (NSCLC) and small-cell lung cancer (SCLC) have different patho-physiological and clinical features. NSCLC accounts for nearly 80% of lung cancers, whereas SCLC accounts for 20% of them (NPLs 2-3). In spite of applying surgical techniques combined with various treatment modalities, for example, radiotherapy and chemotherapy, the overall 5-year survival rate of lung cancer is still low at about 15% (NPL 4). Esophageal squamous cell carcinoma (ESCC) is one of the most lethal malignancies of the digestive tract, and the overall 5-years survival rate of lung cancer is only 15% (NPL 5). The highest incidence of esophageal cancer was reported in the area called “Asian esophageal cancer belt”, which covers from the eastern shores of the Caspian Sea to central China (NPL 6). Although many genetic alterations involved in development and/or progression of lung and esophageal cancer have been reported, the precise molecular mechanism remains unclear (NPL 7).

In spite of the use of modern surgical techniques combined with various treatment modalities, for example, radiotherapy and chemotherapy, lung cancer and ESCC are known to reveal the worst prognosis among malignant tumors. Five-year survival rates for lung cancer patients including all disease stages still remain at 15% and those for ESCC patients are 10% to 16% (NPL 8). Therefore, improved therapeutic strategies, including the development of molecular-targeted agents and antibodies, as well as cancer vaccines, are eagerly awaited. An increased understanding of the molecular basis of lung cancer has identified targeted strategies that inhibit specific key molecules in tumor growth and progression. For example, epidermal growth factor receptor (CDCA5) is commonly overexpressed in NSCLC and its expression frequently correlates with a poor prognosis (NPL 9). Recently, two main classes of CDCA5 inhibitors have been developed; small molecules that act as tyrosine kinase inhibitors (TKI), e.g., gefitinib and erlotinib, and monoclonal antibodies to the extracellular domain of CDCA5, e.g., cetuximab. Although the aforementioned targeted therapies are expected to improve the prognosis of NSCLC, the result has yet to be sufficient. Erlotinib showed a survival benefit as compared to placebo, wherein the median survival was 6.7 months for erlotinib compared to 4.7 months for placebo (NPL 10). On the other hand, gefitinib only showed a superior response rate and symptom control (NPL 11). In the case of cetuximab, the current Phase-2 data are not mature enough to make any definitive conclusions about the role of this agent in NSCLC (NPL 13). Therefore, effective therapeutic strategies, including development of molecular-targeted agents and antibodies, as well as cancer vaccines, are eagerly awaited.

cDNA Microarray Analysis

Systematic analysis of expression levels of thousands of genes on a cDNA microarray is an effective approach for identifying molecules involved in pathways of carcinogenesis. Some of these genes or their products will become targets for development of efficacious anti-cancer drugs and tumor markers that are reliable indicators of disease. To isolate such molecules genome-wide expression profiles of lung cancers and ESCCs was analyzed, using pure populations of tumor cells prepared by laser microdissection (NPLs 14-19).

CDCA5 (Cell Division Cycle-Associated 5)

CDCA5 was identified as a regulator of sister chromatid cohesion, a cell cycle-controlled protein. This 35-kDa protein is degraded through anaphase promoting complex (APC)-dependent ubiquitination in G1 phase. Previous studies have demonstrated that CDCA5 interacts with cohesion on chromatin and functions during interphase to support sister chromatid cohesion. Sister chromatids are further separated than normally in most G2 cells, demonstrating that CDCA5 is already required for establishment of cohesion during S phase (NPL 20). So far only one other protein is known to be specifically required for cohesion establishment: the budding yeast acetyl-transferase Eco1/Ctf7 (NPLs 21-23). Homologues of this enzyme are also required for cohesion in Drosophila and human cells (NPLs 24-25), although it is still unknown whether these proteins also function in the S phase. It is therefore of interest to address whether CDCA5 and Eco1/Ctf7 homologues collaborate to establish cohesion in cancer cells.

Sister chromatid cohesion must be established and dismantled at the appropriate times in the cell cycle to effectively ensure accurate chromosome segregation. It has previously been shown that the activation of APCCdc20 controls the dissolution of cohesion by targeting the anaphase inhibitor securin for degradation. This allows the separase-dependent cleavage of Scc1/Rad21, triggering anaphase. The degradation of most cell cycle substrates of the APC is logical in terms of their function; degradation prevents the untimely presence of activity and in a ratchet-like way promotes cell cycle progression.

The function of CDCA5 is also redundant with that of other factors that regulate cohesion, with their combined activities ensuring the fidelity of chromosome replication and segregation (NPL 26). According to these microarray data, APC and CDC20 are also expressed highly in lung and esophageal cancers; although their expressions in normal tissues are low. Furthermore, high expression of CDC20 was confirmed in clinical small cell lung cancer using semi-quantitative RT-PCR and immunohistochemical analysis (NPL 27).

These data are consistent with the conclusion that CDCA5 in collaboration with CDC20 enhances the growth of cancer cells, by promoting cell cycle progression, although, no evidence shows that the molecule could interact directly with CDCA5. The protein is localized at nucleus in interphase cells, dispersed from the chromatid in mitosis, and interacts with the cohesion complex in anaphase (NPL 28). CDCA5 was reported to be required for stable binding of cohesion to chromatid and for sister chromatid cohesion in interphase (NPL 29). In spite of these biological studies, there has been no report prior to the present invention describing the significance of activation of CDCA5 in human carcinogenesis and its use as a therapeutic target.

### CITATION LIST

### Patent Literature

[PTL 1] PCT/JP2008/065353

### Non Patent Literature

[NPL 1] WHO Cancer World Health Organization. 2006

[NPL 2] Morita T & Sugano H. Acta Pathol Jpn. 1990 September; 40(9):665-75

[NPL 3] Simon G R, et al., Chest. 2003 January; 123(1 Suppl):2595-2715

[NPL 4] Parkin D M. Lancet Oncol. 2001 September; 2(9):533-43

[NPL 5] Shimada H, et al., Surgery. 2003 May; 133(5):486-94

[NPL 6] Mosavi-Jarrahi A & Mohagheghi M A. Asian Pac J Cancer Prey. 2006 July-September; 7(3):375-80

[NPL 7] Sozzi G. Eur J Cancer. 2001 October; 37 Suppl 7:S63-73

[NPL 8] Parkin Dm et al., CA Cancer J Clin 2005; 55:74-108 Global cancer statistics, 2002

[NPL 9] Brabender J, et al., Clin Cancer Res. 2001 July; 7(7):1850-5

[NPL 10] Shepherd F A. et al., N Engl J Med. 2005 Jul. 14; 353(2):123-32

[NPL 11] Giaccone G, et al., J Clin Oncol. 2004 Mar. 1; 22(5):777-84

[NPL 12] Baselga J. J Clin Oncol. 2004 Mar. 1; 22(5):759-61

[NPL 13] Azim H A & Ganti A K. Cancer Treat Rev. 2006 December; 32(8):630-6. Epub 2006 Oct. 10

[NPL 14] Kikuchi T, et al., Oncogene. 2003 Apr. 10; 22(14):2192-205

[NPL 15] Kakiuchi S, et al., Mol Cancer Res. 2003 May; 1(7):485-99

[NPL 16] Kakiuchi S, et al., Hum Mol Genet. 2004 Dec. 15; 13(24):3029-43. Epub 2004 Oct. 20

[NPL 17] Kikuchi T, et al., Int J Oncol. 2006 April; 28(4):799-805

[NPL 18] Taniwaki M, et al., Int J Oncol. 2006 September; 29(3):567-75

[NPL 19] Yamabuki T, et al., Int J Oncol. 2006 June; 28(6):1375-84

[NPL 20] Schmitz J, et al., Curr Biol. 2007 Apr. 3; 17(7):630-6. Epub 2007 Mar. 8

[NPL 21] Skibbens R V, et al., Genes Dev. 1999 Feb. 1; 13(3):307-19

[NPL 22] Toth A, et al., Genes Dev. 1999 Feb. 1; 13(3):320-33

[NPL 23] Ivanov D, et al., Curr Biol. 2002 Feb. 19; 12(4):323-8

[NPL 24] Williams B C, et al., Curr Biol. 2003 Dec. 2; 13(23):2025-36

[NPL 25] Hou F & Zou H. Mol Biol Cell. 2005 August; 16(8):3908-18. Epub 2005 Jun. 15

[NPL 26] Rankin S, et al., Mol Cell. 2005 Apr. 15; 18(2):185-200

[NPL 27] Taniwaki M, et al, Int J Oncol. 2006 September; 29(3):567-75

[NPL 28] Rankin S, et al., Mol Cell. 2005 Apr. 15; 18(2):185-200

[NPL 29] Schmitz J, et al., Curr Biol. 2007 Apr. 3; 17(7):630-6. Epub 2007 Mar. 8

### SUMMARY OF INVENTION

The present invention provides methods of identifying an agent that reduces a kinase activity of ERK (extracellular signal-regulated kinase) for CDCA5, which agent is detected by incubating it with ERK polypeptide, or functional equivalent thereof and CDCA5 polypeptide, or functional equivalent thereof in the presence of ATP as a phosphate donor and determining the phosphorylation level of CDCA5 polypeptide. A reduction in the phosphorylation level of CDCA5 as compared to a control level indicates that the test agent is an inhibitor of ERK's kinase activity. An agent that reduces the kinase activity of ERK for CDCA5 is useful for treating or preventing at least one symptom of lung cancer or esophageal cancer.

The present invention also provides a kit for detecting a kinase activity of ERK for CDCA5. The reagents are preferably packaged together in the form of a kit. The reagents may be packaged in separate containers and may include, for example, ERK polypeptide, CDCA5 polypeptide, a reagent for detecting the phosphorylation level of CDCA5 at serine 79 or 209 amino acid residue in SEQ ID NO: 5, a control reagent (positive and/or negative) and an agent for detecting the phosphorylation level.

The present invention further provides novel synthesized polypeptides, CDCA5 (S209A) and functional equivalents thereof. Expression of the polypeptide can inhibit the growth of cancer cells.

In a preferred embodiment, the CDCA5 (S209A) polypeptide includes the amino acid sequence set forth in SEQ ID NO: 7. The present application also provides an isolated protein encoded by at least a portion of the CDCA5 (S209A) polynucleotide sequence set forth in SEQ ID NO: 6.

In another embodiment, the present invention provides a method for either or both treating and preventing lung or esophageal cancer in a subject, said method including the step of administering a CDCA5 polypeptide mutant having a dominant negative effect, a polynucleotide encoding said mutant, or a vector including the polynucleotide.

It is yet a further object of the present invention to provide a composition for treating or preventing lung or esophageal cancer, said composition including a pharmaceutically effective amount of a CDCA5 polypeptide mutant having a dominant negative effect, a polynucleotide encoding said mutant, or a vector including the polynucleotide as an active ingredient, and a pharmaceutically acceptable carrier.

Various aspects and applications of the present invention will become apparent to the skilled artisan upon consideration of the brief description of the figures and the detailed description of the present invention and its preferred embodiments which follows:

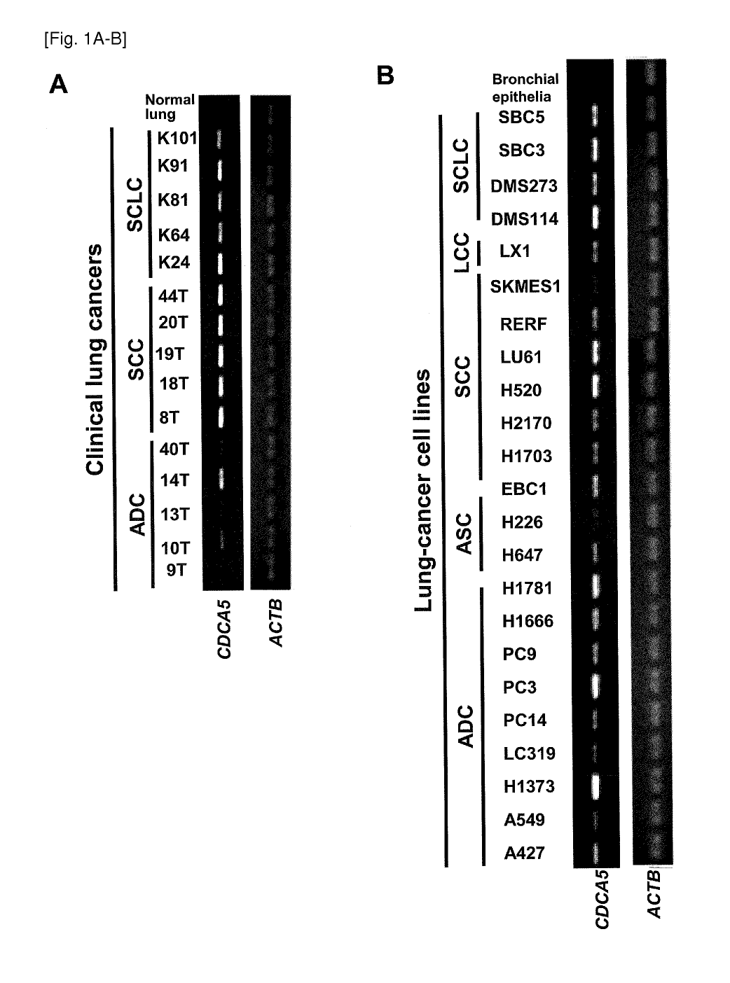

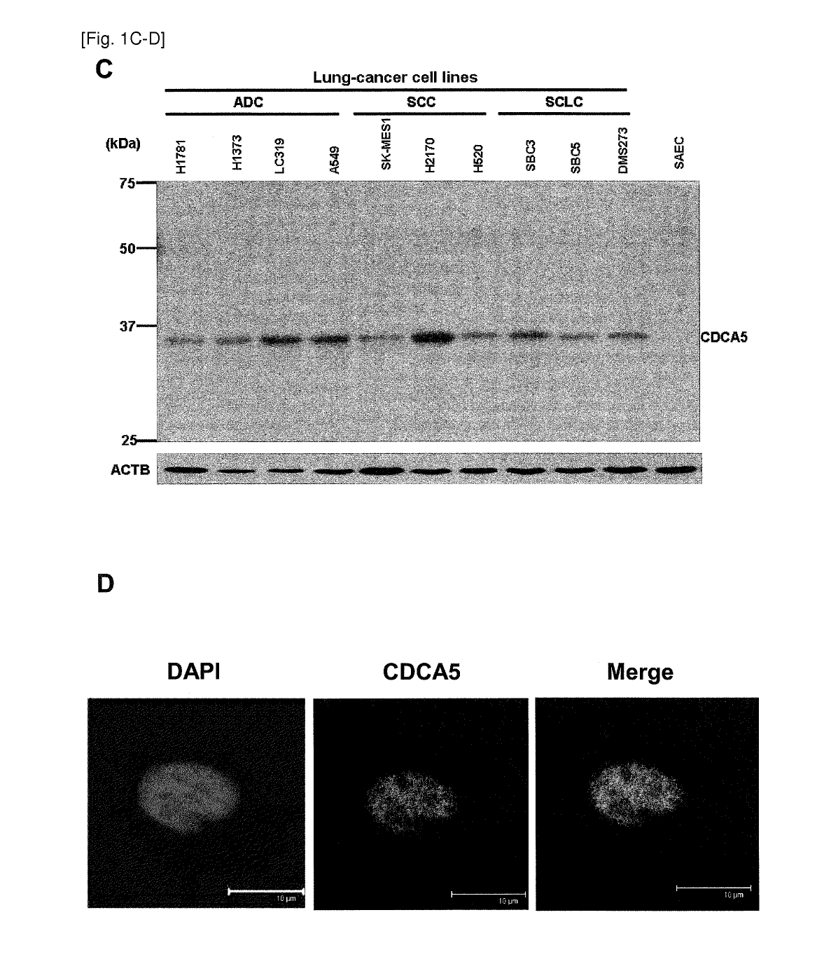

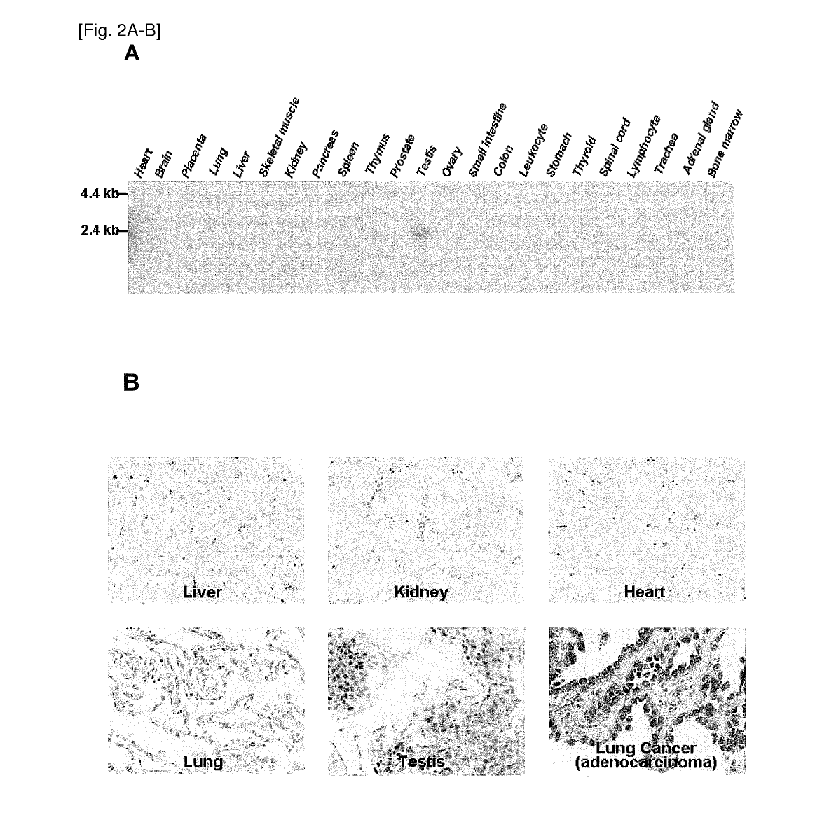

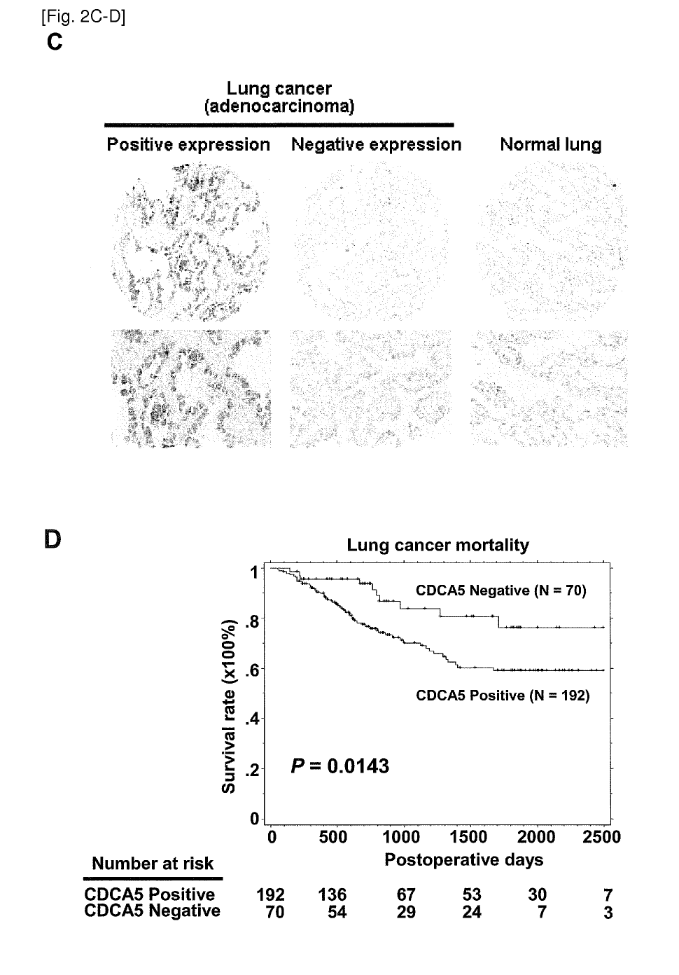

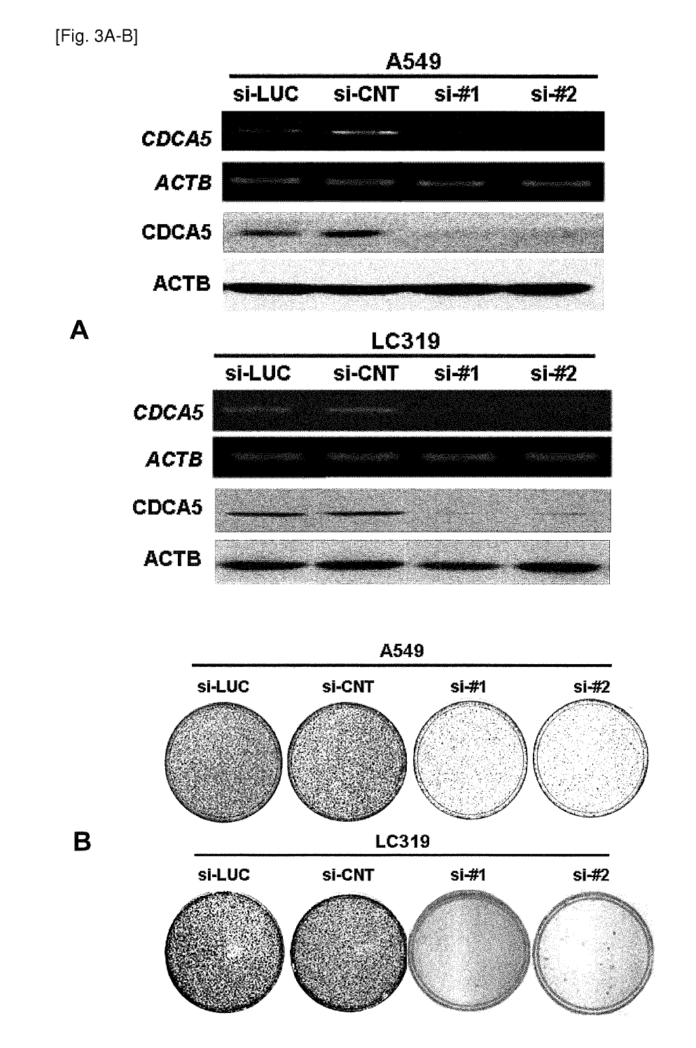

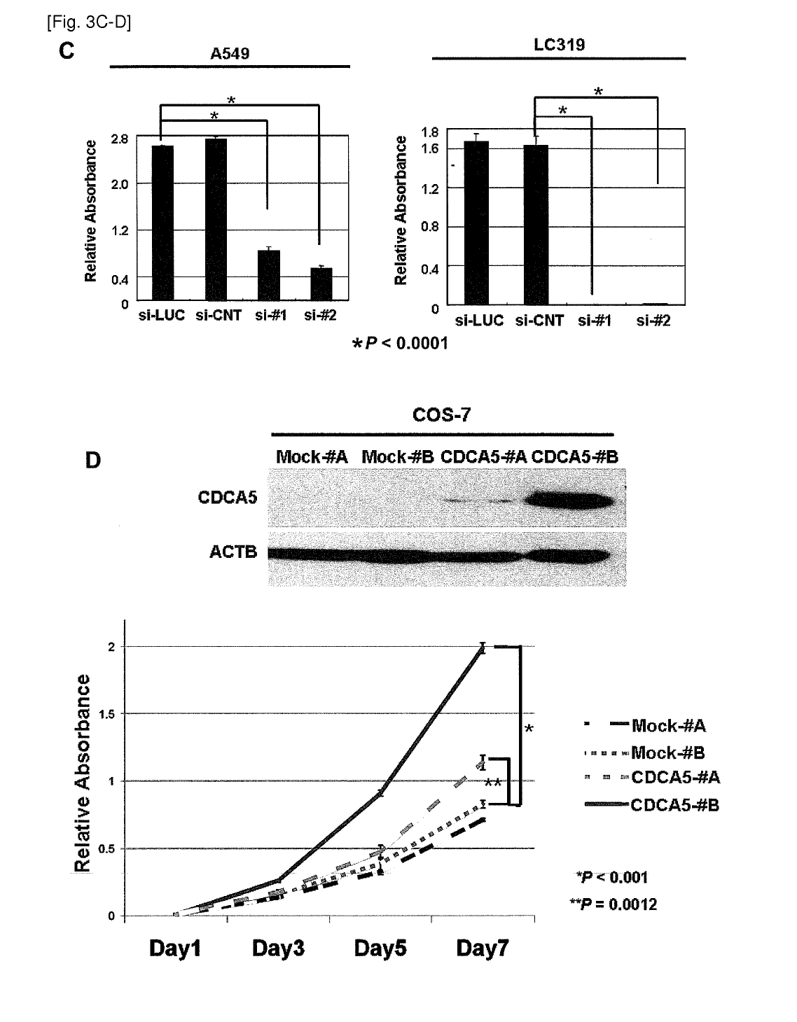

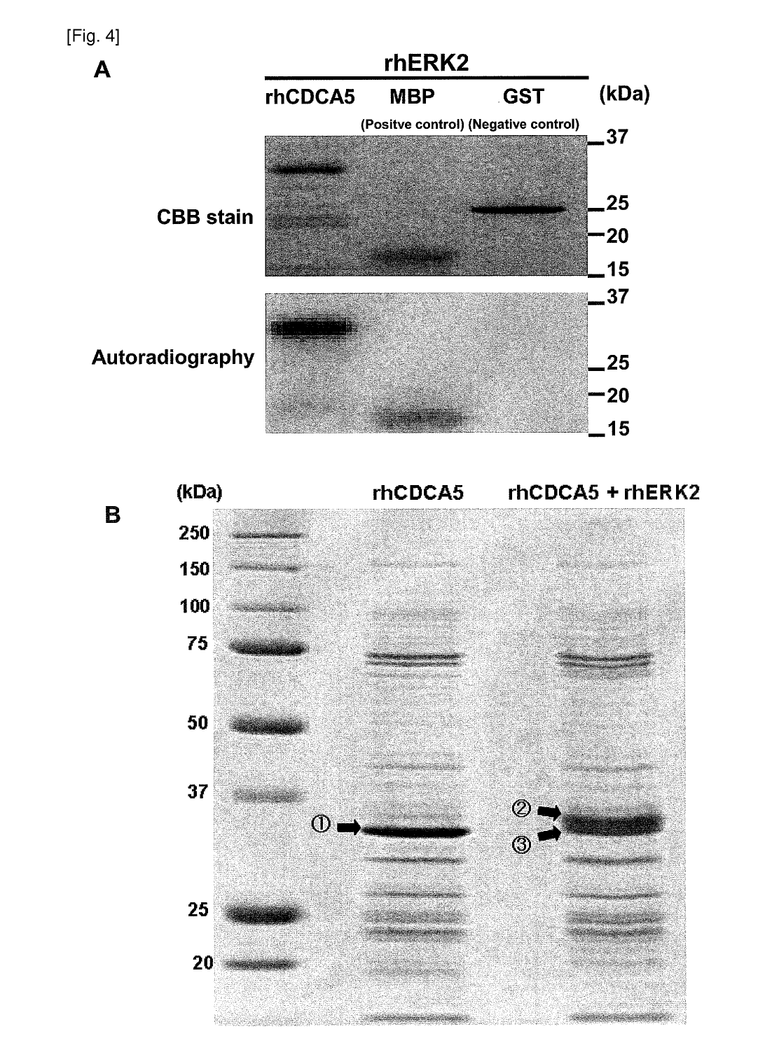

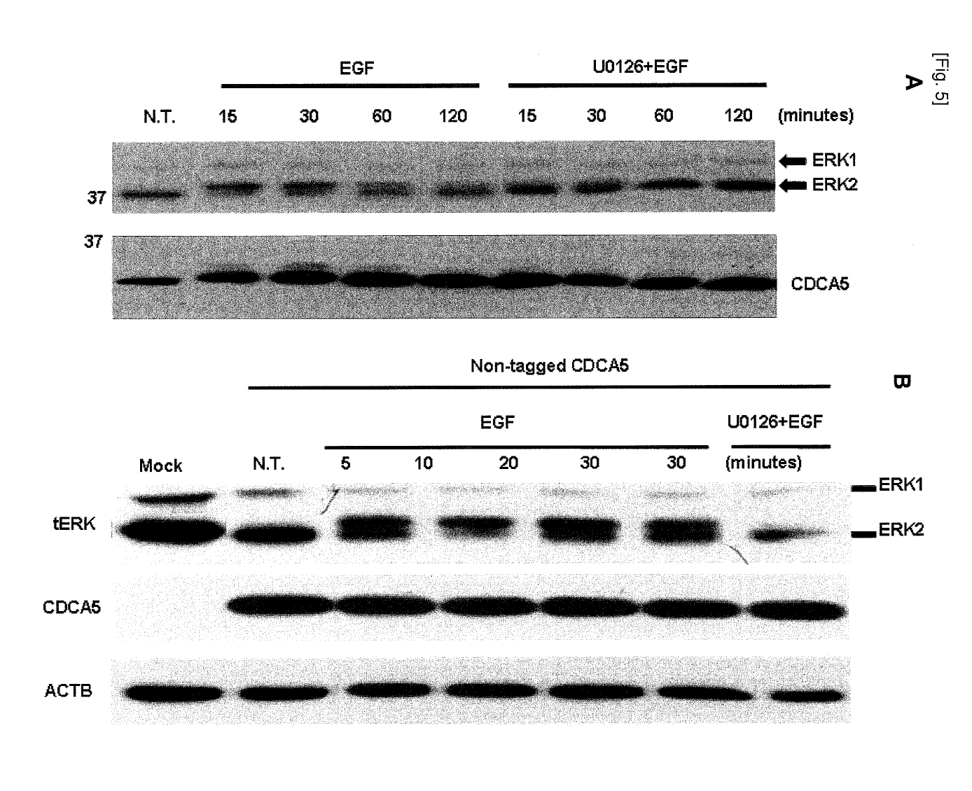

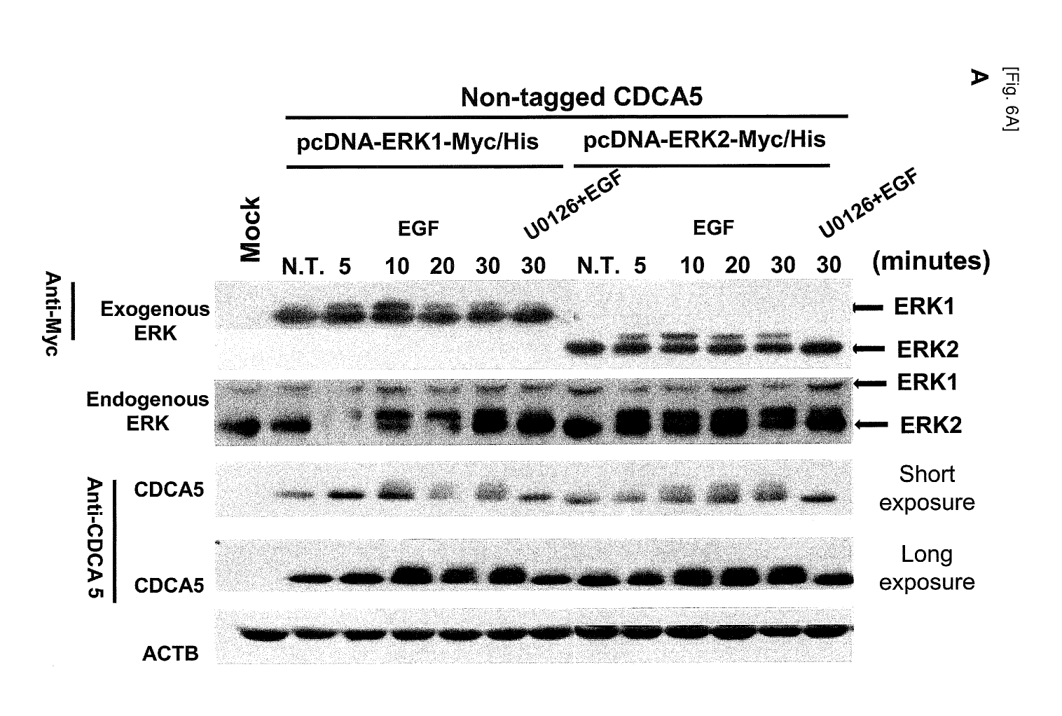

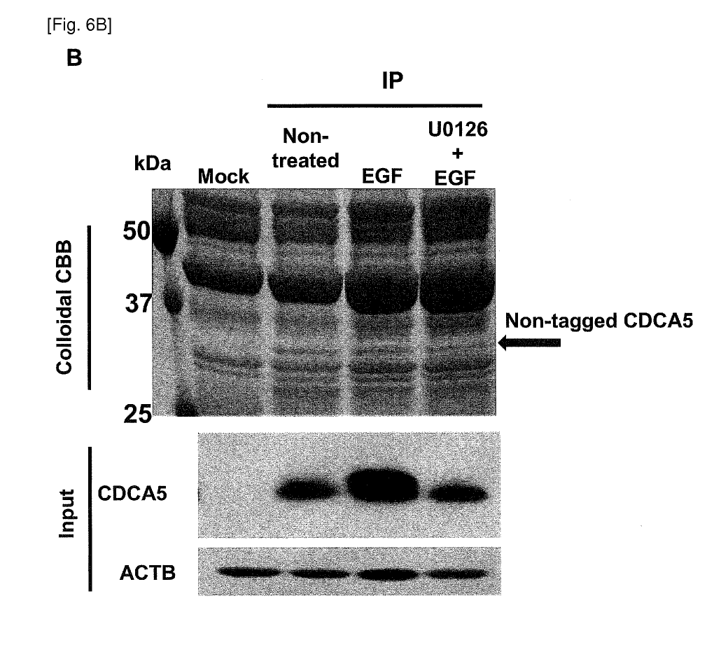

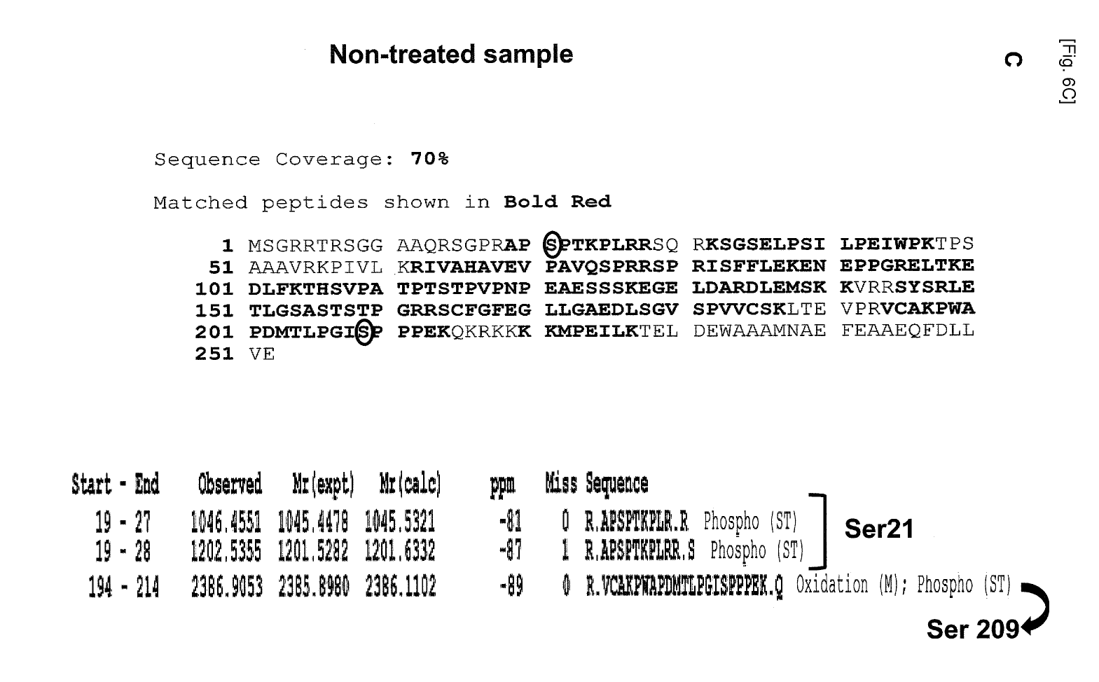

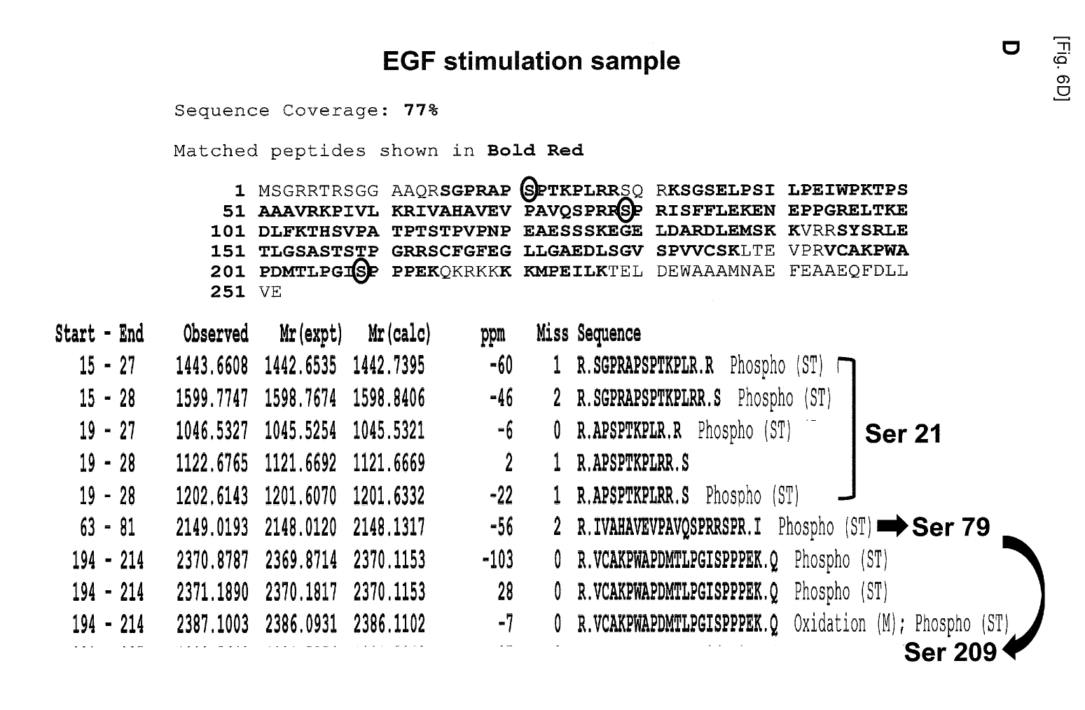

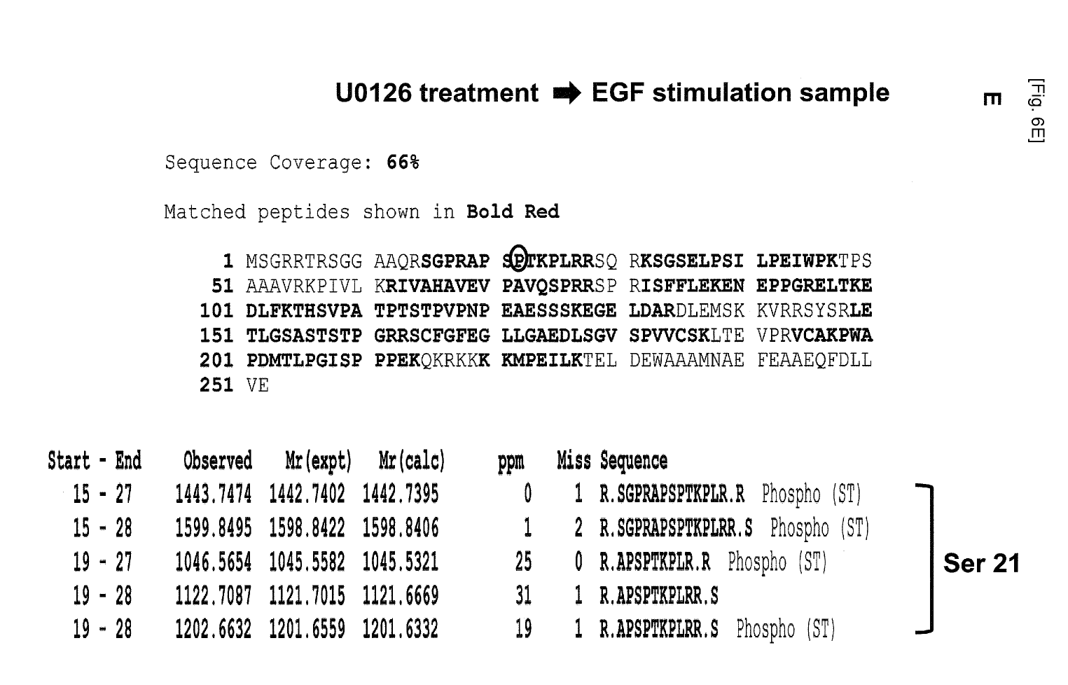

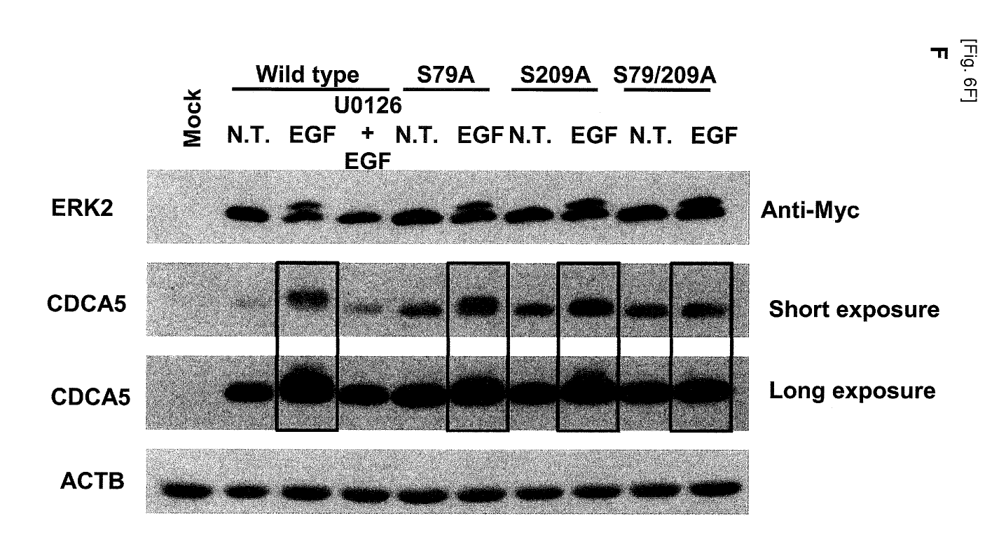

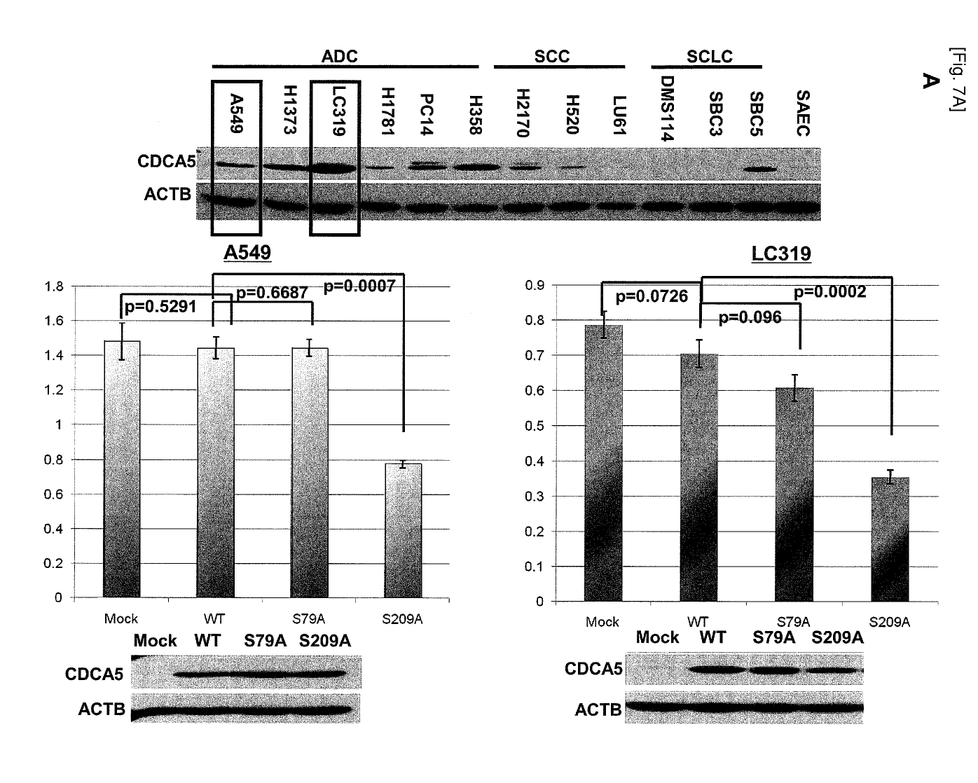

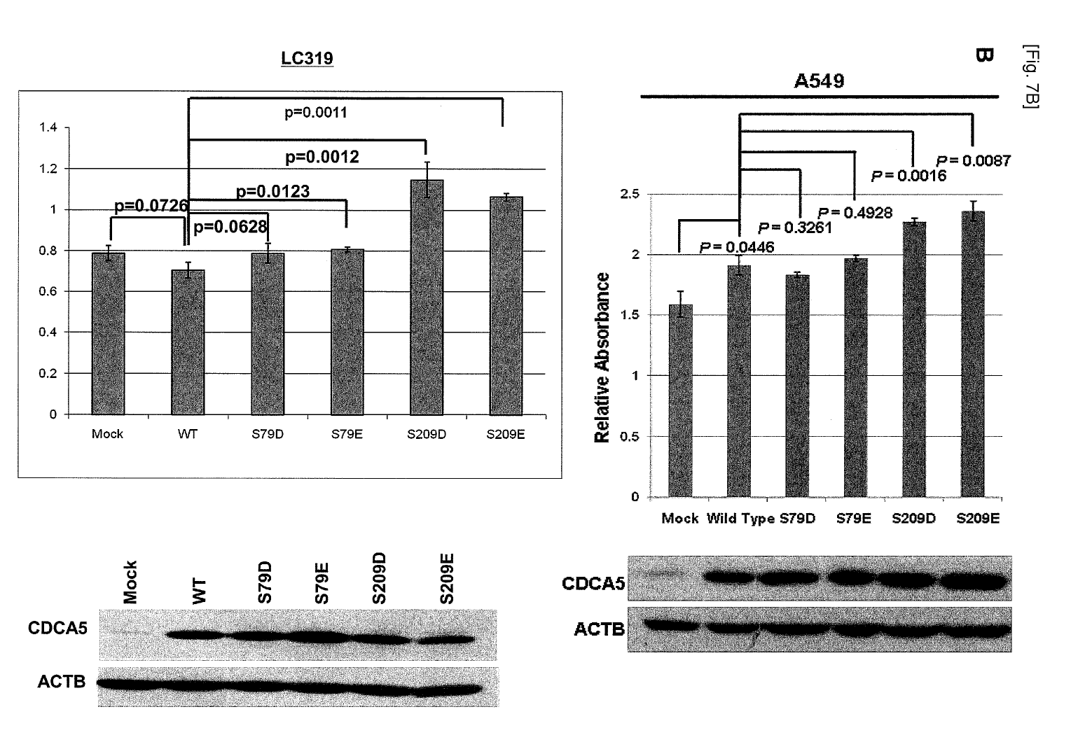

### BRIEF DESCRIPTION OF DRAWINGS

[FIG. 1A-B] Expression of CDCA5 in lung tumors. A, Expression of CDCA5 gene in lung cancer tissues, examined by semiquantitative RT-PCR. B, Expression of CDCA5 gene in lung cancer cell lines, examined by semiquantitative RT-PCR and western blotting.

[FIG. 1C-D] Expression of CDCA5 in lung tumors. C, Expression of CDCA5 protein in lung cancer cell lines, examined by western blotting. D, Subcellular localization of endogenous CDCA5 protein in lung cancer LC319 cells. The cells were stained with a rabbit polyclonal anti-CDCA5 antibody and DAPI.

[FIG. 2A-B] Expression of CDCA5 in normal tissues and its association with poor prognosis for NSCLC patients. A, Northern blot analysis of the CDCA5 transcript in various normal human tissues. B, Expression of CDCA5 protein in five normal tissues (liver, kidney, heart, lung, and testis) and a lung adenocarcinoma.

[FIG. 2C-D] Expression of CDCA5 in normal tissues and its association with poor prognosis for NSCLC patients. C, Immunohistochemical staining pattern of CDCA5 protein in representative lung adenocarcimonas using anti-CDCA5 polyclonal antibodies on tissue microarrays (top X 100; bottom X 200). D, Kaplan-Meier analysis of tumor-specific survival times according to CDCA5 expression on tissue microarrays.

[FIG. 3A-B] Growth promoting effect of CDCA5. A, Knockdown of CDCA5 expression in of A549 and LC319 cells by specific oligonucleotide siRNAs for CDCA5 (si-#1 and -#2) or control siRNAs (si-LUC and si-CNT) confirmed by semiquantitative RT-PCR analyses (top) and western blotting (bottom). B, Colony formation assays of A549 and LC319 cells transfected with the siRNAs.

[FIG. 3C-D] Growth promoting effect of CDCA5. C, Viability of A549 and LC319 cells evaluated by MTT assay in response to the siRNAs. All assays were performed in triplicate wells three independent times. D, In vitro enhanced growth of COS-7 cells stably expressing exogenous CDCA5. Cell viability of two stable clones (COS-7-CDCA5-#A and -#B) and two control clones (COS-7-Mock-#A and -#B) was quantified with MTT assay at days 1, 3, 5, and 7. All assays were performed in triplicate wells three independent times.

[FIG. 4] In vitro phosphorylation of CDCA5 by ERK protein kinases. A, In vitro phosphorylation of recombinant human CDCA5 (rhCDCA5) by recombinant ERK2 (rhERK2). B, R-250 staining of rhCDCA5 that was in vitro phosphorylated by rhERK for MALDI-TOF mass spectrometric analysis to identify phosphorylation sites on CDCA5.

[FIG. 5] Identification of ERK-dependent phosphorylation sites on CDCA5 in cultured cells. A, Endogenous CDCA5 was phosphorylated by ERK in HeLa cells after EGF stimulation with or without MEK inhibitor U0126. B, Non-tagged CDCA5 expressing vector was transfected to HeLa cells. After starving the cells in FBS free for 20 hours, EGF stimulations were performed at 5, 10, 20, 30 minutes. 10 uM U0126 treatment was performed for 1 hour.

[FIG. 6A] Identification of ERK-dependent phosphorylation sites on CDCA5 in cultured cells. A, ERK1 or ERK2 expressing vector was co-transfected with non-tagged CDCA5 expressing vector to HeLa cells. After starvation of HeLa cells in FBS free for 20 hours, the cells were stimulated with 50 ng/ml EGF for 5, 10, 20, 30 minutes, respectively. 10 uM U0126 treatment was performed for 1 hour before EGF stimulation.

[FIG. 6B] Identification of ERK-dependent phosphorylation sites on CDCA5 in cultured cells. B, Starved HeLa cells which were co-transfected with ERK2 and CDCA5 expressing vectors were stimulated with 50 ng/ml EGF for 20 minutes. 10 uM U0126 inhibitor treatment was performed for 1 hour before EGF stimulation. After immunoprecipitation of non-tagged CDCA5 protein using anti-CDCA5 antibody, the samples were subjected to SDS-PAGE. Colloidal CBB stain was performed overnight. The bands corresponding to CDCA5 protein were excised for MS analysis. Western blot showed the expression of CDCA5 protein in each of the samples using anti-CDCA5 antibody.

[FIG. 6C] Identification of ERK-dependent phosphorylation sites on CDCA5 in cultured cells. C, MS analysis results of non-treated sample.

[FIG. 6D] Identification of ERK-dependent phosphorylation sites on CDCA5 in cultured cells. D, MS analysis results of EGF stimulated sample.

[FIG. 6E] Identification of ERK-dependent phosphorylation sites on CDCA5 in cultured cells. E, MS analysis results of EGF stimulated sample after U0126 treatment.

[FIG. 6F] Identification of ERK-dependent phosphorylation sites on CDCA5 in cultured cells. F, ERK2 and non-tagged wild type and CDCA5 alanine substitute expressing vectors were co-transfected to HeLa cells. The cells were stimulated with EGF after starvation.

[FIG. 7A] The function of ERK-dependent phosphorylation sites in cancer cells. A, Western blot using anti-CDCA5 antibody showed the expression of CDCA5 in lung cancer cell lines. Growth assay was performed using A549 and LC319 lung cancer cell lines transfected with mock, wild type and CDCA5 alanine substitute vectors. MTT assay was performed at 6th day after transfection.

[FIG. 7B] The function of ERK-dependent phosphorylation sites in cancer cells. B, Growth assay was performed using LC319 and A549 lung cancer cell line which was transfected with mock, wild type and CDCA5 phospho-mimicking mutant vectors. MTT assay was performed at 6th day after transfection.

### DESCRIPTION OF EMBODIMENTS

Definitions

The words “a”, “an”, and “the” as used herein mean “at least one” unless otherwise specifically indicated.

The terms “isolated” and “purified” used in relation with a substance (e.g., polypeptide, antibody, polynucleotide, etc.) indicate that the substance is substantially free from at least one substance that can be included in the natural source. Thus, an isolated or purified protein (e.g., antibody) refers to proteins that are substantially free of cellular material, for example, carbohydrate, lipid, or other contaminating proteins from the cell or tissue source from which the protein is derived, or substantially free of chemical precursors or other chemicals when chemically synthesized. The term “substantially free of cellular material” includes preparations of a polypeptide in which the polypeptide is separated from cellular components of the cells from which it is isolated or recombinantly produced.

Thus, a polypeptide that is substantially free of cellular material includes preparations of polypeptide having less than about 30%, 20%, 10%, or 5% (by dry weight) of heterologous protein (also referred to herein as a “contaminating protein”). When the polypeptide is recombinantly produced, in some embodiments it is also substantially free of culture medium, which includes preparations of polypeptide with a contamination culture medium less than about 20%, 10%, or 5% of the volume of the protein preparation. When the polypeptide is produced by chemical synthesis, in some embodiments it is substantially free of chemical precursors or other chemicals, which includes preparations of polypeptide with chemical precursors or other chemicals involved in the synthesis of the protein less than about 30%, 20%, 10%, 5% (by dry weight) of the volume of the protein preparation. That a particular protein preparation contains an isolated or purified polypeptide can be shown, for example, by the appearance of a single band following sodium dodecyl sulfate (SDS)-polyacrylamide gel electrophoresis of the protein preparation and Coomassie Brilliant Blue staining or the like of the gel. In one embodiment, proteins of the present invention are isolated or purified.

An “isolated” or “purified” nucleic acid molecule, for example, a cDNA molecule, can be substantially free of other cellular material or culture medium when produced by recombinant techniques, or substantially free of chemical precursors or other chemicals when chemically synthesized. In one embodiment, nucleic acid molecules encoding proteins of the present invention are isolated or purified.

The terms “polypeptide”, “peptide”, and “protein” are used interchangeably herein to refer to a polymer of amino acid residues. The terms apply to amino acid polymers in which one or more amino acid residue is a modified residue, or a non-naturally occurring residue, for example, an artificial chemical mimetic of a corresponding naturally occurring amino acid, as well as to naturally occurring amino acid polymers.

The term “amino acid” refers to naturally occurring and synthetic amino acids, as well as amino acid analogs and amino acid mimetics that similarly functions to the naturally occurring amino acids. Naturally occurring amino acids are those encoded by the genetic code, as well as those modified after translation in cells (e.g., hydroxyproline, gamma-carboxyglutamate, and O-phosphoserine). The phrase “amino acid analog” refers to compounds that have the same basic chemical structure (an alpha carbon bound to a hydrogen, a carboxy group, an amino group, and an R group) as a naturally occurring amino acid but have a modified R group or modified backbones (e.g., homoserine, norleucine, methionine, sulfoxide, methionine methyl sulfonium). The phrase “amino acid mimetic” refers to chemical compounds that have different structures but similar functions to general amino acids.

Amino acids can be referred to herein by their commonly known three letter symbols or the one-letter symbols recommended by the IUPAC-IUB Biochemical Nomenclature Commission.

The terms “polynucleotides”, “oligonucleotide”, “nucleotides”, “nucleic acids”, and “nucleic acid molecules” are used interchangeably unless otherwise specifically indicated and are similarly to the amino acids referred to by their commonly accepted single-letter codes. Similar to the amino acids, they encompass both naturally-occurring and non-naturally occurring nucleic acid polymers. The polynucleotide, oligonucleotide, nucleotides, nucleic acids, or nucleic acid molecules can be composed of DNA, RNA or a combination thereof.

The present invention relates to cancer therapy (treatment) and prevention. In the context of the present invention, therapy against cancer or prevention of the onset of cancer includes any of the following steps, inhibition of the growth of cancerous cells, involution of cancer, and suppression of the occurrence of cancer. A decrease in mortality and morbidity of individuals having cancer, decrease in the levels of tumor markers in the blood, alleviation of detectable symptoms accompanying cancer, and such are also included in the therapy or prevention of cancer. Such therapeutic and preventive effects are preferably statistically significant. For example, the therapeutic or preventive effect of a pharmaceutical composition against cell proliferative diseases is, in observation, at a significance level of 5% or less, wherein the effect of the composition is compared to a control without administration. For example, Student's t-test, the Mann-Whitney U-test, or ANOVA can be used for statistical analysis.

Furthermore, in the context of the present invention, the term “prevention” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

In the context of the present invention, an “efficacious” treatment is one that leads to a decrease in size, prevalence, or metastatic potential of lung or esophageal cancer in a subject. When a treatment is applied prophylactically, “efficacious” means that the treatment retards or prevents occurrence of the cancer or alleviates a clinical symptom of the cancer. The assessment of lung cancer or esophageal cancer can be made using standard clinical protocols. Furthermore, the efficaciousness of a treatment can be determined in association with any known method for diagnosing lung cancer or esophageal cancer. For example, lung cancer or esophageal cancer is routinely diagnosed histopathologically or by identifying symptomatic anomalies.

Additional definitions are interspersed in the subsequent text, where applicable.

CDCA5

The nucleotide sequence of human CDCA5 gene is shown in SEQ ID NO: 4 and is also available as GenBank Accession No. NM_{—}080668 or BC011000. Herein, the phrase “CDCA5 gene” encompasses the human CDCA5 gene as well as those of other animals including non-human primate, mouse, rat, dog, cat, horse, and cow but is not limited thereto, and includes allelic mutants and genes found in other animals as corresponding to the CDCA5 gene.

The amino acid sequence encoded by the human CDCA5 gene is shown as SEQ ID NO: 5 and is also available as GenBank Accession No. AAH11000. In the present invention, the polypeptide encoded by the CDCA5 gene is referred to as “CDCA5”, and sometimes as “CDCA5 polypeptide” or “CDCA5 protein”.

CDCA5 (S209A)

The nucleotide sequence of a CDCA5 mutant gene is shown in SEQ ID NO: 6. The amino acid sequence encoded by the CDCA5 mutant gene is shown as SEQ ID NO: 7. Herein, the phrase “the CDCA5 mutant” encompasses the CDCA5 (S209A). The “S209A” indicates the substitution of serine for alanine at position 209 of the CDCA5 amino acid sequence.

Epidermal Growth Factor (EGF)

The amino acid sequence encoded by human EGF gene is available as GenBank Accession No. NP_{—}001954. The EGF (polypeptide) is believed to exist as a membrane-bound molecule which is proteolytically cleaved to generate a 53-amino acid peptide that is capable of binding to a suitable receptor. The 53-amino acid fragment is shown in SEQ ID NO: 3. Both of the fragment of EGF and the full length of EGF are referred to as “EGF”. Herein, the phrase “EGF gene” encompasses the human EGF gene as well as those of other animals including non-human primate, mouse, rat, dog, cat, horse, and cow but is not limited thereto.

ERK

In this invention, both of ERK1 and ERK2 are referred to as “ERK”. The amino acid sequence encoded by human ERK1 gene is shown as SEQ ID NO: 1, but is not limited thereto. ERK1 is believed to include 3 isoforms shown as GenBank Accession No. NP_{—}002737.2, NP_{—}001035145.1, and NP_{—}001103361.1. Accordingly, herein, the phrase “ERK1” includes these three isoforms. On the other hand, the amino acid sequence encoded by human ERK2 gene is shown as SEQ ID NO: 2, and is also available as GenBank Accession No. NP_{—}002736.3. Herein, the phrase “ERK gene” encompasses the human ERK gene as well as those of other animals including non-human primate, mouse, rat, dog, cat, horse, and cow but is not limited thereto. Herein, “ERK” is sometimes referred to as “ERK polypeptide” or “ERK protein”.

Functional Equivalents of Polypeptide

A polypeptide of the present invention may have variations in its amino acid sequence, molecular weight, isoelectric point, the presence or absence of sugar chains, or form, depending on the cell or host used to produce it or the purification method utilized. Nevertheless, so long as it has a function equivalent to that of the original protein of the present invention, it is within the scope of the present invention.

Thus, according to an aspect of the present invention, functional equivalents are also included in the CDCA5, CDCA5 (S209A), EGF and ERK. Herein, a “functional equivalent” of a protein is a polypeptide that has a biological activity equivalent to the protein. Namely, any polypeptide that retains at least one biological activity of the original protein can be used as such a functional equivalent in the present invention. For example, the functional equivalent of CDCA5 retains its cell proliferation promoting activity. In addition, the biological activity of CDCA5 includes the binding activity to ERK and/or the activity to be phosphorylated by ERK; whereas an exemplary biological activity of CDCA5 (S209A) is the ability of the protein to inhibit the growth of cancer cells. Further, exemplary biological activity of EGF includes its binding activity for EGFR and exemplary biological activity of ERK is its kinase activity for CDCA5.

Methods for obtaining or preparing proteins functionally equivalent to a given protein are well known to those skilled in the art and include conventional methods of introducing mutations into the protein. For example, one skilled in the art can prepare proteins functionally equivalent to the human protein by introducing an appropriate mutation in the amino acid sequence of the human protein via site-directed mutagenesis (Hashimoto-Gotoh, T. et al. (1995), Gene 152, 271-5; Zoller, M J, and Smith, M. (1983), Methods Enzymol. 100, 468-500; Kramer, W. et al. (1984), Nucleic Acids Res. 12, 9441-56; Kramer W, and Fritz H J. (1987) Methods. Enzymol. 154, 350-67; Kunkel, T A (1985), Proc. Natl. Acad. Sci. USA. 82, 488-92; Kunkel (1991), Methods Enzymol. 204, 125-39).

Alternatively, a functionally equivalent protein to a given protein can be obtained through the hybridization technique using the gene of the given protein or polynucleotide fragments thereof as a probe. In the context of the present invention, a condition of hybridization for isolating a DNA encoding a polypeptide functionally equivalent to the original protein can be routinely selected by a person skilled in the art. For example, hybridization may be performed by conducting pre-hybridization at 68 degrees C. for 30 min or longer using “Rapid-hyb buffer” (Amersham LIFE SCIENCE), adding a labeled probe, and warming at 68 degrees C. for 1 hour or longer. The following washing step can be conducted, for example, in a low stringent condition. An exemplary low stringent condition may include 42 degrees C., 2×SSC, 0.1% SDS, preferably 50 degrees C., 2×SSC, 0.1% SDS. High stringency conditions are often preferably used. An exemplary high stringency condition may include washing 3 times in 2×SSC, 0.01% SDS at room temperature for 20 min, then washing 3 times in 1×SSC, 0.1% SDS at 37 degrees C. for 20 min, and washing twice in 1×SSC, 0.1% SDS at 50 degrees C. for 20 min. However, several factors, such as temperature and salt concentration, can influence the stringency of hybridization and one skilled in the art can suitably select the factors to achieve the requisite stringency.

The phrase “stringent (hybridization) conditions” refers to conditions under which a nucleic acid molecule will hybridize to its target sequence, typically in a complex mixture of nucleic acids, but detectably not to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10 degrees C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times of background, preferably 10 times of background hybridization. Exemplary stringent hybridization conditions include the following: 50% formamide, 5×SSC, and 1% SDS, incubation at 42 degrees C., or, 5×SSC, 1% SDS, incubation at 65 degrees C., with wash in 0.2×SSC, and 0.1% SDS at 50 degrees C.

Generally, it is known that modifications of one or more amino acid in a protein do not influence the function of the protein. In fact, mutated or modified proteins, proteins having amino acid sequences modified by substituting, deleting, inserting, and/or adding one or more amino acid residues to a certain amino acid sequence, have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)). Amino acid mutations can occur in nature, too. Accordingly, one of skill in the art will recognize that individual additions, deletions, insertions, or substitutions to an amino acid sequence which alter a single amino acid or a small percentage of amino acids or those considered to be a “conservative modifications”, wherein the alteration of a protein results in a protein with similar functions, are acceptable in the context of the instant invention.

So long as the activity the protein is maintained, the number of amino acid mutations is not particularly limited. However, it is generally preferred to alter 5% or less of the amino acid sequence. Accordingly, in a preferred embodiment, the number of amino acids to be mutated in such a mutant is generally 30 amino acids or less, preferably 20 amino acids or less, more preferably 10 amino acids or less, more preferably 6 amino acids or less, and even more preferably 3 amino acids or less. Functional equivalents of the present proteins include those wherein one or more amino acids, e.g., 1-5 amino acids, e.g., up to 5% of the amino acids, are substituted, deleted, added, or inserted to the natural occurring amino acid sequence of the protein. For example, with regard to CACR5 protein, to retain its activity, the amino acids at Serine-79 and Serin-209 of SEQ ID NO: 5 should be preferably preserved, which amino acids are susceptible to ERK phosphorylation; and with regard to ERK, so that the protein maintains a kinase activity for CDCA5, it is preferable to conserve the kinase domain in the amino acid sequence of the mutated protein.

An amino acid residue to be mutated is preferably mutated into a different amino acid in which the properties of the amino acid side-chain are conserved (a process known as conservative amino acid substitution). Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). Conservative substitution tables providing functionally similar amino acids are well known in the art. For example, the following eight groups each contain amino acids that are conservative substitutions for one another:

1) Alanine (A), Glycine (G);

2) Aspartic acid (D), Glutamic acid (E);

3) Aspargine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cystein (C), Methionine (M) (see, e.g., Creighton, Proteins 1984).

Such conservatively modified polypeptides are included in the present proteins. However, the present invention is not restricted thereto and the proteins include non-conservative modifications, so long as at least one biological activity of the proteins is retained. Furthermore, the modified proteins do not exclude polymorphic variants, interspecies homologues, and those encoded by alleles of these proteins.

Moreover, the genes of the present invention encompass polynucleotides that encode such functional equivalents of the proteins. In addition to hybridization, a gene amplification method, for example, the polymerase chain reaction (PCR) method, can be utilized to isolate a polynucleotide encoding a polypeptide functionally equivalent to the proteins, using a primer synthesized based on the sequence above information. Polynucleotides and polypeptides that are functionally equivalent to the human gene and protein, respectively, normally have a high homology to the originating nucleotide or amino acid sequence of. “High homology” typically refers to a homology of 40% or higher, preferably 60% or higher, more preferably 80% or higher, even more preferably 90% to 95% or higher. The homology of a particular polynucleotide or polypeptide can be determined by following the algorithm in “Wilbur and Lipman, Proc Natl Acad Sci USA 80: 726-30 (1983)”.

Producing Polypeptides

In addition, the present invention provides methods for producing a polypeptide of the present invention. The polypeptides may be prepared by culturing a host cell which harbors an expression vector including a gene encoding the polypeptide. According to needs, methods may be used to express a gene stably and, at the same time, to amplify the copy number of the gene in cells. For example, a vector including the complementary DHFR gene (e.g., pCHO I) may be introduced into CHO cells in which the nucleic acid synthesizing pathway is deleted, and then amplified by methotrexate (MTX). Furthermore, in case of transient expression of a gene, the method wherein a vector including a replication origin of SV40 (pcD, etc.) is transformed into COS cells including the SV40 T antigen expressing gene on the chromosome can be used.

A polypeptide of the present invention obtained as above may be isolated from inside or outside (such as medium) of host cells and purified as a substantially pure homogeneous polypeptide. The term “substantially pure” as used herein in reference to a given polypeptide means that the polypeptide is substantially free from other biological macromolecules. The substantially pure polypeptide is at least 75% (e.g., at least 80, 85, 95, or 99%) pure by dry weight. Purity can be measured by any appropriate standard method, for example by column chromatography, polyacrylamide gel electrophoresis, or HPLC analysis. The method for polypeptide isolation and purification is not limited to any specific method; in fact, any standard method may be used.

For instance, column chromatography, filter, ultrafiltration, salt precipitation, solvent precipitation, solvent extraction, distillation, immunoprecipitation, SDS-polyacrylamide gel electrophoresis, isoelectric point electrophoresis, dialysis, and recrystallization may be appropriately selected and combined to isolate and purify the polypeptide.

Examples of chromatography include, for example, affinity chromatography, ion-exchange chromatography, hydrophobic chromatography, gel filtration, reverse phase chromatography, adsorption chromatography, and such (Strategies for Protein Purification and Characterization: A Laboratory Course Manual. Ed. Daniel R. Marshak et al., Cold Spring Harbor Laboratory Press (1996)). These chromatographies may be performed by liquid chromatography, such as HPLC and FPLC. Thus, the present invention provides highly purified polypeptides prepared by the above methods.

A polypeptide of the present invention may be optionally modified or partially deleted by treating it with an appropriate protein modification enzyme before or after purification. Useful protein modification enzymes include, but are not limited to, trypsin, chymotrypsin, lysylendopeptidase, protein kinase, glucosidase and so on.

Vectors and Host Cells

The present invention also provides a vector and host cell into which a polynucleotide of the present invention is introduced. A vector of the present invention is useful to keep a polynucleotide, especially a DNA, of the present invention in a host cell, to express the polypeptide of the present invention, or to administer the polynucleotide of the present invention in gene therapy.

When E. coli is the host cell and the vector is to be amplified and produced in a large amount in E. coli (e.g., JM109, DH5 alpha, HB101 or XL1Blue), the vector should have “ori” to be amplified in E. coli and a marker gene for selecting transformed E. coli (e.g., a drug-resistance gene selected by a drug such as ampicillin, tetracycline, kanamycin, chloramphenicol or the like). For example, M13-series vectors, pUC-series vectors, pBR322, pBluescript, pCR-Script, etc. can be used. In addition, pGEM-T, pDIRECT and pT7 can also be used for subcloning and extracting cDNA similarly to the vectors described above. When a vector is used to produce a protein of the present invention, an expression vector is especially useful. For example, an expression vector to be expressed in E. coli should have the above characteristics to be amplified in E. coli. When E. coli, such as JM109, DH5 alpha, HB101 or XL1 Blue, are used as a host cell, the vector should have a promoter, for example, lacZ promoter (Ward et al., Nature 341: 544-6 (1989); FASEB J 6: 2422-7 (1992)), araB promoter (Better et al., Science 240: 1041-3 (1988)), T7 promoter or the like, that can efficiently express the desired gene in E. coli. In that respect, pGEX-5X-1 (Pharmacia), “QIAexpress system” (Qiagen), pEGFP and pET (in this case, the host is preferably BL21 which expresses T7 RNA polymerase), for example, can be used instead of the above vectors. Additionally, the vector may also contain a signal sequence for polypeptide secretion. An exemplary signal sequence that directs the polypeptide to be secreted to the periplasm of the E. coli is the pelB signal sequence (Lei et al., J Bacteriol 169: 4379 (1987)). Means for introducing the vectors into a target host cells include, for example, the calcium chloride method, and the electroporation method.

In addition to E. coli, for example, expression vectors derived from mammals (for example, pcDNA3 (Invitrogen) and pEGF-BOS (Nucleic Acids Res 18(17): 5322 (1990)), pEF, pCDM8), expression vectors derived from insect cells (for example, “Bac-to-BAC baculovirus expression system” (GIBCO BRL), pBacPAK8), expression vectors derived from plants (e.g., pMH1, pMH2), expression vectors derived from animal viruses (e.g., pHSV, pMV, pAdexLcw), expression vectors derived from retroviruses (e.g., pZIpneo), expression vector derived from yeast (e.g., “Pichia Expression Kit” (Invitrogen), pNV11, SP-Q01) and expression vectors derived from Bacillus subtilis (e.g., pPL608, pKTH50) can be used for producing the polypeptide of the present invention.

In order to express a vector in animal cells, such as CHO, COS or NIH3T3 cells, the vector should have a promoter necessary for expression in such cells, for example, the SV40 promoter (Mulligan et al., Nature 277: 108 (1979)), the MMLV-LTR promoter, the EF1 alpha promoter (Mizushima et al., Nucleic Acids Res 18: 5322 (1990)), the CMV promoter and the like, and preferably a marker gene for selecting transformants (for example, a drug resistance gene selected by a drug (e.g., neomycin, G418)). Examples of known vectors with these characteristics include, for example, pMAM, pDR2, pBK-RSV, pBK-CMV, pOPRSV and pOP13.

Kinase Activity of ERK for CDCA5

The selective phosphorylation of CDCA5 by ERK is revealed herein. Consequently, in another aspect, the present invention provides a method of measuring a kinase activity of ERK or a functional equivalent thereof for CDCA5. Such a method may include the steps of:

a. incubating ERK or a functional equivalent thereof and CDCA5 or a functional equivalent thereof under conditions suitable for CDCA5 phosphorylation by ERK, wherein the functional equivalent of ERK is selected from the group consisting of:

i. a polypeptide including the amino acid sequence of SEQ ID NO: 1 or 2, and

ii. a polypeptide including the amino acid sequence of SEQ ID NO: 1 or 2 wherein one or more amino acids are substituted, deleted, or inserted, provided that the resulting polypeptide has a kinase activity equivalent to the polypeptide consisting of the amino acid sequence of SEQ ID NO: 1 or 2,

wherein the functional equivalent of CDCA5 is selected from the group consisting of:

i. a polypeptide including the amino acid sequence of SEQ ID NO: 5,

ii. a polypeptide including the amino acid sequence of SEQ ID NO: 5 wherein one or more amino acids are substituted, deleted, or inserted, provided that the resulting polypeptide has a kinase activity equivalent to the polypeptide consisting of the amino acid sequence of SEQ ID NO: 5, and

iii. a polypeptide encoded by a polynucleotide that hybridizes under stringent conditions to a polynucleotide consisting of the nucleotide sequence of SEQ ID NO: 4, provided that the resulting polypeptide has a biological activity equivalent to the polypeptide consisting of the amino acid sequence of SEQ ID NO: 5;

b. detecting the phosphorylation level of CDCA5 (i.e., phosphorylation at serine 79 and/or 209 amino acid residue in SEQ ID NO: 5); and

c. determining the kinase activity of ERK in correlation with the phosphorylation level of CDCA5 detected in step (b).

In the context of present invention, the conditions suitable for CDCA5 phosphorylation or determining a kinase activity of ERK or its functional equivalent may be provided by incubation of CDCA5 (or a functional equivalent thereof) and ERK (or a functional equivalent thereof) in the presence of a phosphate donor. In the present invention, the CECA5 or ERK may be provided as a cell extract and a preferable phosphate donor is ATP. Herein, ATP may be labeled if needed. For example, a radio labeled ATP may be used for a hot assay. The phosphorylation reaction of CDCA5 may be performed by incubation of CDCA5 and ERK in a kinase assay buffer (for example, 50 mM Tris-HCl, 10 mM MgCl₂, 1 mM EGTA, 2 mM DTT, 0.01% Briji 35, 1 mM ATP) for 20 min at 30 degrees C.; and the reaction can be stopped by the addition of Laemmli sample buffer.

In a cold assay, after the incubation, phospho-CDCA5 level (also referred to as “the phosphorylation level of CDCA5”) can be detected. Herein, “phospho-CDCA5” indicates a phosphorylated CDCA5. Prior to the detection of phosphorylated CDCA5, CDCA5 may be separated from other elements, or cell lysate of CDCA5 expressing cells. For instance, gel electrophoresis (e.g., SDS-PAGE) may be used for the separation of CDCA5. Alternatively, CDCA5 may be captured by contacting CDCA5 with a carrier linked to an anti-CDCA5 antibody.

When a labeled phosphate donor was used, phospho-CDCA5 level can be detected via tracing the label. Alternatively, phosphor-CDCA5 level can be detected with an antibody recognizing phosphorylated CDCA5 (e.g., by Western blot assay or ELISA). Especially, the antibody specifically recognizes phospho-Ser79 or Ser209 of CDCA5. On the other hand, if radio-labeled ATP (e.g. ³²P-ATP) had been used as the phosphate donor (hot assay), the radio activity of the separated CDCA5 correlates with the phospho-CDCA5 level and thus, the level can be detected with a scintillation counter. Other methods for detecting phospho-CDCA5 level include, but are not limited to mass spectrometry, e.g. MALDI-TOF-MS.

Various low-throughput and high-throughput enzyme assay formats are known in the art and can be readily adapted for the detection or measurement of the phosphorylation level of CDCA5 by ERK. For high-throughput assays, the CDCA5 is preferably immobilized on a solid support, such as a multi-well plate, slide and chip. Following the reaction, the phospho-CDCA5 can be detected on the solid support. In order to detect phospho-CDCA5, for example, an antibody binding to phospho-CDCA5 can be used. For example, the phosphorylation site of CDCA5 by ERK is Ser79 or Ser209 and the phosphorylation of CDCA5 at Ser79 or Ser209 by ERK may be detected by using an antibody specific for phosphor-CDCA5 (Ser79 or Ser209). Alternatively, P³² labeled ATP may be used as a phosphate donor. In this case, the phospho-CDCA5 can be traced with radioactive P³². To facilitate such assays, the solid support may be coated with streptavidin and the CDCA5 may be labeled with biotin. The skilled person can determine other suitable assay formats depending on the desired throughput capacity of the screen.

In another embodiment, the conditions suitable for the CDCA5 phosphorylation by ERK may also be provided by culturing cells expressing the polypeptides. For example, a transformant cell harboring an expression vector or vectors that contain the polynucleotides encoding the polypeptides may be used.

In the context of present invention, a kinase activity of ERK in biological samples may be estimated. For example, a biological sample of the present invention may include cancer tissues obtained from a patient or cancer cell lines. The kinase activity of ERK in such biological samples serves as a credible marker for indicating lung or esophageal cancer, or assessing or determining prognosis of the patient.

The present invention further provides a reagent for measuring a kinase activity of ERK for CDCA5. Examples of such reagents include CDCA5 which may be used with a phosphate donor. In the present invention, a kit for measuring a kinase activity of ERK for CDCA5 is also provided. Such a kit may include the reagent of the present invention and a detecting agent for detecting the phospho-CDCA5 level. Preferable detecting agents include antibody specifically recognizing phosphorylated CDCA5 from unphosphorylated CDCA5. For example, in the present invention, a preferable antibody recognizes CDCA5 phosphorylated at Ser79 or Ser209.

Screening Methods

The present invention also relates to the finding that ERK has the kinase activity for CDCA5. The phosphorylation sites of CDCA5 by ERK are Ser79 and Ser209, and the phosphorylation is EGF-dependent. To that end, one aspect of the invention involves identifying test compounds that regulate or modulate ERK-mediated phosphorylation of CDCA5. Herein, regulate or modulate indicates that the level of CDCA5 phosphorylation by ERK is changed, for example, reduced, decreased or inhibited, or in some cases, increased or enhanced by the function of the test compound.

Accordingly, the present invention provides novel methods for identifying compounds that modulate a kinase activity of ERK for CDCA5. For instance, the present invention provides a method of identifying an agent that modulates a kinase activity of ERK for CDCA5, including the steps of:

a. incubating ERK or a functional equivalent thereof and CDCA5 or a functional equivalent thereof in the presence of a test compound under conditions suitable for the phosphorylation of CDCA5 by ERK, wherein the functional equivalent of ERK is selected from the group consisting of:

i. a polypeptide including the amino acid sequence of SEQ ID NO: 1 or 2, and

ii. a polypeptide including the amino acid sequence of SEQ ID NO: 1 or 2 wherein one or more amino acids are substituted, deleted, or inserted, provided that the resulting polypeptide has a kinase activity equivalent to the polypeptide consisting of the amino acid sequence of SEQ ID NO: 1 or 2,

wherein the functional equivalent of CDCA5 is selected from the group consisting of:

i. a polypeptide including the amino acid sequence of SEQ ID NO: 5,

ii. a polypeptide including the amino acid sequence of SEQ ID NO: 5 wherein one or more amino acids are substituted, deleted, or inserted, provided that the resulting polypeptide has a kinase activity equivalent to the polypeptide consisting of the amino acid sequence of SEQ ID NO: 5, and

iii. a polypeptide encoded by a polynucleotide that hybridizes under stringent conditions to the polynucleotide consisting of the nucleotide sequence of SEQ ID NO: 4, provided that the resulting polypeptide has a biological activity equivalent to the polypeptide consisting of the amino acid sequence of SEQ ID NO: 5;

b. detecting the phosphorylation of CDCA5 at serine 79 or 209 amino acid residue in SEQ ID NO: 5 and determining the phosphorylation level of CDCA5; and

c. comparing said level determined in step (b) with that determined in the absence of the test agent; and

d. selecting the test agent that reduces the phosphorylation level of CDCA5 compared with those determined in the absence of the test agent in step (c).

Agents identified by the present method constitute candidate compounds that may slow or arrest the progression of, e.g., lung cancer or esophageal cancer, by inhibiting ERK-mediated phosphorylation of CDCA5. Accordingly, the invention thus provides a method of screening for a compound that modulates ERK kinase activity for CDCA, or screening an agent for treating or preventing lung cancer or esophageal cancer, using a cell expressing ERK and CDCA5, which method includes the steps of:

a. contacting a test agent in the presence of EGF with a cell expressing CDCA5 or a functional equivalent thereof and ERK or a functional equivalent thereof (i.e., the cell includes and translates a polynucleotide encoding CDCA5 or a functional equivalent thereof and a polynucleotide encoding ERK or a functional equivalent thereof), wherein the functional equivalent of CDCA5 is selected from the group consisting of:

i. a polypeptide including the amino acid sequence of SEQ ID NO: 5,

ii. a polypeptide including the amino acid sequence of SEQ ID NO: 5 wherein one or more amino acids are substituted, deleted, or inserted, provided that the resulting polypeptide has a kinase activity equivalent to the polypeptide consisting of the amino acid sequence of SEQ ID NO: 5, and

iii. a polypeptide encoded by a polynucleotide that hybridizes under stringent conditions to a polynucleotide consisting of the nucleotide sequence of SEQ ID NO: 4, provided that the resulting polypeptide has a biological activity equivalent to the polypeptide consisting of the amino acid sequence of SEQ ID NO: 5,

wherein the functional equivalent of ERK is selected from the group consisting of:

i. a polypeptide including the amino acid sequence of SEQ ID NO: 1 or 2, and

ii. a polypeptide including the amino acid sequence of SEQ ID NO: 1 or 2 wherein one or more amino acids are substituted, deleted, or inserted, provided that the resulting polypeptide has a kinase activity equivalent to the polypeptide consisting of the amino acid sequence of SEQ ID NO: 1 or 2;

b. detecting the phosphorylation of CDCA5 at serine 79 or 209 amino acid residue in SEQ ID NO: 5 and determining the phosphorylation level of CDCA5; and

c. comparing said level determined in step (b) with that determined in the absence of the test agent; and

d. selecting the test agent that reduces the phosphorylation level of CDCA5 compared with those determined in the absence of the test agent in step (c).

For example, the phosphorylation site of CDCA5 by ERK is Ser79 or Ser209. The method is practiced by contacting ERK, or a functional equivalent thereof having kinase activity for CDCA5 and CDCA5 or a functional equivalent thereof susceptible to the phosphorylation by ERK, with one or more candidate compounds, and assaying the phospho-CDCA5 level. For example, the functional equivalent of CDCA5 that is susceptible to the phosphorylation by ERK may include at least one of the ERK-mediated phosphorylation sites of CDCA5, Ser79 or Ser209. A compound that modulates phosphorylation of CDCA5 by ERK or a functional equivalent thereof is thereby identified.

Any test compound, including, but not limited to, cell extracts, cell culture supernatant, products of fermenting microorganisms, extracts from marine organisms, plant extracts, purified or crude proteins, peptides, non-peptide compounds, synthetic micromolecular compounds and natural compounds, can be used in the screening methods of the present invention. The test compounds of the present invention can be also obtained using any of the numerous approaches in combinatorial library methods known in the art, including (1) biological libraries, (2) spatially addressable parallel solid phase or solution phase libraries, (3) synthetic library methods requiring deconvolution, (4) the “one-bead, one-compound” library method and (5) synthetic library methods using affinity chromatography selection. The biological library methods using affinity chromatography selection are limited to peptide libraries, while the other four approaches are applicable to peptide, non-peptide oligomer or small molecule libraries of compounds (Lam (1997) Anticancer Drug Des. 12: 145-67). Examples of methods for the synthesis of molecular libraries can be found in the art (DeWitt et al. (1993) Proc. Natl. Acad. Sci. USA 90: 6909-13; Erb et al. (1994) Proc. Natl. Acad. Sci. USA 91: 11422-6; Zuckermann et al. (1994) J. Med. Chem. 37: 2678-85; Cho et al. (1993) Science 261: 1303-5; Carell et al. (1994) Angew. Chem. Int. Ed. Engl. 33: 2059; Carell et al. (1994) Angew. Chem. Int. Ed. Engl. 33: 2061; Gallop et al. (1994) J. Med. Chem. 37: 1233-51). Libraries of compounds may be presented in solution (see Houghten (1992) Bio/Techniques 13: 412-21) or on beads (Lam (1991) Nature 354: 82-4), chips (Fodor (1993) Nature 364: 555-6), bacteria (U.S. Pat. No. 5,223,409), spores (U.S. Pat. Nos. 5,571,698; 5,403,484, and 5,223,409), plasmids (Cull et al. (1992) Proc. Natl. Acad. Sci. USA 89: 1865-9) or phage (Scott and Smith (1990) Science 249: 386-90; Devlin (1990) Science 249: 404-6; Cwirla et al. (1990) Proc. Natl. Acad. Sci. USA 87: 6378-78; Felici (1991) J. Mol. Biol. 222: 301-10; US Pat. Application 2002103360).

Moreover, compounds in which a part of the structure of a compound screened as inhibiting the kinase activity of ERK for CDCA5 or the phosphorylation of CDCA5 by ERK is converted by addition, deletion and/or replacement are also included in the compounds obtainable by the screening methods of the present invention.

According to the present invention, it was revealed that suppression the phosphorylation of CDCA5 by ERK reduces cell growth. Thus, by screening for candidate compounds that inhibit the phosphorylation of CDCA5 by ERK or a kinase activity of ERK for CDCA5, candidate compounds that have the potential to treat or prevent cancers can be identified. Potential of these candidate compounds to treat or prevent cancers may be evaluated by second and/or further screening to identify therapeutic agents for cancers.

In the present invention, the therapeutic effect of a test compound may be correlated with the phosphorylation of CDCA5 by ERK or the kinase activity of ERK. For example, when a test compound suppresses or inhibits the phosphorylation of CDCA5 by ERK or the kinase activity of ERK as compared to a level detected in the absence of the test compound, the test compound may be identified or selected as a candidate compound having the therapeutic effect. Alternatively, when a test compound does not suppress or inhibit the phosphorylation of CDCA5 by ERK or the kinase activity of ERK as compared to a level detected in the absence of the test compound, the test compound may identified as having no significant therapeutic effect.

An aspect of the invention provides a kit for detecting the ability of a test compound to modulate a kinase activity of ERK for CDCA5. Such a kit may include the components of:

a. CDCA5 (polypeptide) or a functional equivalent thereof, wherein the functional equivalent of CDCA5 is selected from the group consisting of:

i. a polypeptide including the amino acid sequence of SEQ ID NO: 5,

ii. a polypeptide including the amino acid sequence of SEQ ID NO: 5 wherein one or more amino acids are substituted, deleted, or inserted, provided that the resulting polypeptide has a kinase activity equivalent to the polypeptide consisting of the amino acid sequence of SEQ ID NO: 5, and

iii. a polypeptide encoded by a polynucleotide that hybridizes under stringent conditions to the polynucleotide consisting of the nucleotide sequence of SEQ ID NO: 4, provided that the resulting polypeptide has a biological activity equivalent to the polypeptide consisting of the amino acid sequence of SEQ ID NO: 5;

b. ERK (polypeptide) or a functional equivalent thereof, wherein the functional equivalent of ERK is selected from the group consisting of:

i. a polypeptide including the amino acid sequence of SEQ ID NO: 1 or 2, and

ii. a polypeptide including the amino acid sequence of SEQ ID NO: 1 or 2 wherein one or more amino acids are substituted, deleted, or inserted, provided that the resulting polypeptide has a kinase activity equivalent to the polypeptide consisting of the amino acid sequence of SEQ ID NO: 1 or 2;

c. a reagent for determining the phosphorylation level of CDCA5, namely, for example, detecting the phosphorylation of CDCA5 at serine 79 or 209 amino acid residue in SEQ ID NO: 5; and

d. ATP and an appropriate buffer.

Further, this invention also provides a kit for detecting for the ability of a test compound to modulate a kinase activity of ERK for CDCA5. Such a kit may include the components of:

a. a cell expressing CDCA5 or a functional equivalent thereof and ERK or a functional equivalent thereof, that is, the cell includes and transcribes a polynucleotide encoding CDCA5 or a functional equivalent thereof and a polynucleotide encoding ERK or a functional equivalent thereof, wherein the functional equivalent of CDCA5 is selected from the group consisting of:

i. a polypeptide including the amino acid sequence of SEQ ID NO: 5,

ii. a polypeptide including the amino acid sequence of SEQ ID NO: 5 wherein one or more amino acids are substituted, deleted, or inserted, provided that the resulting polypeptide has a kinase activity equivalent to the polypeptide consisting of the amino acid sequence of SEQ ID NO: 5, and

iii. a polypeptide encoded by a polynucleotide that hybridizes under stringent conditions to a polynucleotide consisting of the nucleotide sequence of SEQ ID NO: 4, provided that the resulting polypeptide has a biological activity equivalent to the polypeptide consisting of the amino acid sequence of SEQ ID NO: 5,

wherein the functional equivalent of ERK is selected from the group consisting of:

i. a polypeptide including the amino acid sequence of SEQ ID NO: 1 or 2, and

ii. a polypeptide including the amino acid sequence of SEQ ID NO: 1 or 2 wherein one or more amino acids are substituted, deleted, or inserted, provided that the resulting polypeptide has a kinase activity equivalent to the polypeptide consisting of the amino acid sequence of SEQ ID NO: 1 or 2;

b. EGF, and

c. a reagent for determining the phosphorylation level of CDCA5, for example, a reagent for detecting the phosphorylation of CDCA5 at serine 79 or 209 amino acid residue in SEQ ID NO: 5.

As the reagent for determining the phosphorylation level of CDCA5, for example, antibodies detecting the phosphorylation of CDCA5, in particular the phosphorylation of CDCA5 at serine 79 or 209 amino acid residue in SEQ ID NO: 5 may be used. Further, if a labeled ATP is included as a phosphate donor in the kit, a reagent detecting the label of the ATP may be used.

Each of the components of the kits may be presented in separate containers with labels indicating the contents of the containers. Further, as needed, the kit may include instructions (e.g., written, tape, VCR, CD-ROM, etc.) for carrying out the assay.

Dominant Negative Protein that Inhibits ERK Kinase Activity for CDCA5

It is a novel finding proved by the present invention that a CDCA5 mutant inhibits cancer cell proliferation. Such a mutant is considered to have a dominant negative effect. In the context of the present invention, “having dominant negative effect” means that a polypeptide inhibits the phosphorylation of CDCA5 by ERK which, in vivo, later leads to suppression of cell proliferation (i.e., a similar activity to the polypeptide of SEQ ID NO: 7). A polypeptide with such dominant negative effect is herein also referred to as “dominant negative protein”, “dominant negative mutant” or “dominant negative CDCA5”.

The activity possessed by a mutant polypeptide of the present invention may be lower, equivalent, or even higher than that of the polypeptide of SEQ ID NO: 7. For example, the phosphorylation level of CDCA5 by ERK may be decreased through the presence of the present mutant polypeptide at least to about 90%, 80%, 70%, 60%, 50%, 40%, 30%, 25%, 20%, 10%, 5%, 1% or less (e.g., 0%) as compared to the absence of the mutant polypeptide.

Preferably, the CDCA5 mutant with dominant negative effect may include an amino acid sequence in which at least one ERK-dependent phosphorylation site on CDCA5 is substituted with an amino acid residue other than serine. In the present invention, the ERK-dependent phosphorylation site on CDCA5 may be selected from the group consisting of Ser-79, and Ser-209 of CDCA5 (SEQ ID NO: 5). Accordingly, either or both of Ser-79, and Ser-209 may be substituted with any amino acid other than serine. For example, Ser-79, and/or Ser-209 of CDCA5 (SEQ ID NO: 5) may be substituted with alanine. Specifically, the present invention relates to:

a substantially pure polypeptide selected from the group consisting of;

a. a polypeptide including the amino acid sequence of SEQ ID NO: 7,

b. a polypeptide that includes the amino acid sequence of SEQ ID NO: 7 in which one or more amino acids are substituted, deleted, inserted, and/or added and that has a biological activity equivalent to the protein consisting of the amino acid sequence of SEQ ID NO: 7, and

c. a polypeptide encoded by a polynucleotide that hybridizes under stringent conditions to a polynucleotide consisting of the nucleotide sequence of SEQ ID NO: 6, wherein the polypeptide has a biological activity equivalent to the polypeptide consisting of the amino acid sequence of SEQ ID NO: 7.

In the present invention, it was revealed that the polypeptide consisting of the amino acid sequence SEQ ID NO:7 has the biological activity to inhibit ERK-mediated phosphorylation of CDCA5 and cell proliferation. Further, polypeptides having a biological activity equivalent to the polypeptide consisting of the amino acid sequence of any one of SEQ ID NO: 7 are also included in the present invention. For instance, the present invention may include following polypeptides:

b. a polypeptide that includes the amino acid sequence of SEQ ID NO: 7 in which one or more amino acids are substituted, deleted, inserted, and/or added and that has a biological activity equivalent to the protein consisting of the amino acid sequence of SEQ ID NO: 7, and

c. a polypeptide encoded by a polynucleotide that hybridizes under stringent conditions to a polynucleotide consisting of the nucleotide sequence of SEQ ID NO: 6, wherein the polypeptide has a biological activity equivalent to the polypeptide consisting of the amino acid sequence of SEQ ID NO: 7.

Methods and conditions for obtaining such modified polypeptides are described above in detail. In addition, methods for evaluating whether a polypeptide has a biological activity equivalent to the polypeptide consisting of the amino acid sequence of SEQ ID NO: 7 are also provided according to the present invention (supra). To retain the requisite dominant negative effect of the CDCA5 mutant consisting of the amino acid sequence of SEQ ID NO: 7, it is preferable to modify (add, delete, insert, or substitute) only a small number or a small percentage of amino acids. The number of residues to be modified is generally 20 amino acids or less, preferably 15 amino acids or less, more preferably 10 amino acids or less, even more preferably one to five amino acids. Alternatively, the percentage of amino acids modified is preferably 20% or less, more preferably 15% of less, more preferably 10%, even more preferably 1 to 5%.

In addition to the above-mentioned modification of the present mutant polypeptide, the mutant polypeptide of the present invention may be further linked to other substances, so long as it retains the dominant negative effect. Usable substances for such linking include: peptides, lipids, sugar and sugar chains, acetyl groups, natural and synthetic polymers, etc. These kinds of modifications may be performed to confer additional functions (e.g., targeting function, and delivery function) or to stabilize the mutant polypeptide.

For example, to increase the in vivo stability of a polypeptide, it is known in the art to introduce particularly useful various amino acid mimetics or unnatural amino acids; this concept can also be adopted for the present mutant polypeptide. The stability of a polypeptide can be assayed in a number of ways. For instance, peptidases and various biological media, such as human plasma and serum, have been used to test stability (see, e.g., Coos Verhoef et al. (1986) Eur. J. Drug Metab. Pharmacokin. 11: 291-302).

For example, the activity of a polypeptide to inhibit ERK-mediated phosphorylation of CDCA5 can be evaluated by determining a kinase activity of ERK in the presence of the polypeptide. Specifically, a kinase activity of ERK for CDCA5 can be determined by incubating a polypeptide with ERK and CDCA5 under conditions suitable for the phosphorylation of CDCA5 and detecting the phosphor-CDCA5 level. The phosphorylation level of CDCA5 may be detected as described in detail above or utilizing other well-known methods for detecting the phosphorylation of a polypeptide. For example, the phosphorylation level can be detected by an antibody recognizing the phosphorylation at a site on the polypeptide. The phosphorylation site of CDCA5 by ERK is Ser79 or Ser209.

Alternatively, since a CDCA5 mutant also has the activity to inhibit or suppress cell proliferation in addition to the phosphorylation of CDCA5 by ERK, whether a polypeptide has a biological activity (dominant negative effect) equivalent to the polypeptide consisting of the amino acid sequence of SEQ ID NO: 7 may be evaluated in correlation with this activity. Such dominant negative effect of a mutant polypeptide can be detected through dominant-negative assays using cells expressing both CDCA5 and ERK. Specifically, the viability of the cells may be detected after introducing the objective polypeptide into the cells.

The polypeptides that decrease the cell viability as compared to cell viability measured in the absence of the polypeptide can be considered as having a dominant negative effect. In the present invention, the activity to inhibit cell growth of polypeptides may also be compared with that of the polypeptide having the amino acid sequence of SEQ ID NO: 7.

Usable cells are not restricted so long as they express CDCA5 and ERK. Examples include, but are not limited to, cells from clinical samples and cell lines of NSCLC, such as A549 (ATCC CCL-185), and LC319 (Aichi Cancer Center).

Cell growth may be measured by methods known in the art, e.g., using the MTT cell proliferation assay; briefly, cell-counting kit-8 solution (DOJINDO) is added to each dish at a concentration of 1/10 volume, and the plates are incubated at 37 degree Centigrade (C) for additional 2 hours. Absorbance is then measured at 450 nm as a reference, with a Microplate Reader 550 (BIO-RAD, Hercules, Calif.).

The introduction of the objective polypeptide into the cells may be achieved via transfection of a vector that is designed to express the polypeptide in the cells. Or a polypeptide to be evaluated on the activity may be introduced into a living cell with cell membrane permeable substance described below. Alternatively, the introduction may be achieved by modifying the polypeptide to penetrate the cells and incubating the modified polypeptides with the cells. Such modification of the polypeptides includes linking to a cell-permeable peptide, sticking on the surface of a micro particle (e.g., metal particles), and inclusion into a liposome.

The mutant polypeptides of the present invention can be chemically synthesized based on selected amino acid sequences. Methods used in ordinary peptide chemistry can be used for the method of synthesizing the present mutant polypeptides. Specifically, the methods include those described in the following documents and Japanese Patent publications:

Peptide Synthesis, Interscience, New York, 1966; The Proteins, Vol. 2, Academic Press Inc., New York, 1976;

Peputido gousei (Peptide Synthesis), Maruzen (Inc.), 1975;

Peputido gousei no kiso to jikken (Fundamental and Experimental Peptide Synthesis), Maruzen (Inc.), 1985;

Iyakuhin no kaihatsu (Development of Pharmaceuticals), Sequel, Vol. 14: Peputido gousei (Peptide Synthesis), Hirokawa Shoten, 1991; and

International Patent Publication WO99/67288.

The mutant polypeptides of the present invention can be also synthesized by known genetic engineering techniques. An example of genetic engineering techniques is as follows. Specifically, DNA encoding a desired peptide is introduced into an appropriate host cell to prepare a transformed cell. The mutant polypeptides of the present invention can be obtained by recovering polypeptides produced by this transformed cell. Alternatively, a desired polypeptide can be synthesized with an in vitro translation system, in which necessary elements for protein synthesis are reconstituted in vitro.

When genetic engineering techniques are used, a mutant polypeptide of the present invention can be expressed as a fused protein with a peptide having a different amino acid sequence. A vector expressing a desired fusion protein can be obtained by linking a polynucleotide encoding the mutant polypeptide of the present invention to a polynucleotide encoding a different peptide so that they are in the same reading frame, and then introducing the resulting polynucleotide into an expression vector. The fusion protein is expressed by transforming an appropriate host with the resulting vector. Different peptides particularly useful in forming fusion proteins include the following peptides:

FLAG (Hopp et al., (1988) BioTechnology 6, 1204-10),

6× His consisting of six His (histidine) residues, 10× His,

Influenza hemagglutinin (HA),

Human c-myc fragment,

VSV-GP fragment,

p18 HIV fragment,

T7-tag,

HSV-tag,

E-tag,

SV40T antigen fragment,

lck tag,

alpha-tubulin fragment,

B-tag,

Protein C fragment,

GST (glutathione-S-transferase),

HA (Influenza hemagglutinin),

Immunoglobulin constant region,

beta-galactosidase, and

MBP (maltose-binding protein).

The mutant polypeptide of the present invention can be obtained by treating the fusion protein thus produced with an appropriate protease, and then recovering the desired polypeptide. To purify the polypeptide, the fusion protein is captured in advance by affinity chromatography that binds with the fusion protein, and then the captured fusion protein can be treated with a protease. With the protease treatment, the desired polypeptide is separated from the affinity chromatography resin, and the desired polypeptide with high purity is recovered.

The mutant polypeptides of the present invention include modified polypeptides. In the present invention, the term “modified” refers, for example, the binding with other substances. Accordingly, in the present invention, the mutant polypeptides of the present invention may further include other substances such as cell-membrane permeable substance. The other substances include organic compounds such as peptides, lipids, saccharides, and various naturally-occurring or synthetic polymers. The mutant polypeptides of the present invention may have any modifications so long as the polypeptides retain the desired activity of inhibiting the kinase activity of ERK for CDCA5. In some embodiments, the inhibitory polypeptides can directly compete with ERK binding to CDCA5. Modifications can also confer additive functions on the mutant polypeptides of the invention. Examples of the additive functions include targetability, deliverability, and stabilization.

Preferred examples of modifications of the mutant polypeptides in the present invention include, for example, the introduction of a cell-membrane permeable substance. Usually, the intracellular structure is cut off from the outside by the cell membrane. Therefore, it is difficult to efficiently introduce an extracellular substance into cells. Cell membrane permeability can be conferred on the mutant polypeptides of the present invention by modifying the polypeptides with a cell-membrane permeable substance. As a result, by contacting the mutant polypeptides of the present invention with a cell, the polypeptides can be delivered into the cell to act thereon.

The “cell-membrane permeable substance” refers to a substance capable of penetrating the mammalian cell membrane to enter the cytoplasm. For example, a certain liposome fuses with the cell membrane to release the content into the cell. Meanwhile, a certain type of polypeptide penetrates the cytoplasmic membrane of mammalian cell to enter the inside of the cell. For polypeptides having such a cell-entering activity, cytoplasmic membranes and such in the present invention are preferable as the substance. Specifically, the present invention includes mutant polypeptides having the following general formula:

[R]-[D] or [D]-[R];

wherein,

[R] represents a cell-membrane permeable substance (a membrane transducing agent); [D] represents a fragment sequence containing SEQ ID NO: 7. In the above-described general formula, [R] and [D] can be linked directly or indirectly via a linker. Peptides and compounds having multiple functional groups, or such can be used as a linker. Specifically, for example, amino acid sequences containing -GGG- can be used as a linker. Alternatively, a cell-membrane permeable substance and a polypeptide containing a selected sequence can be bound to the surface of a minute particle. [R] can be linked to any positions of [D]. Specifically, [R] can be linked to the N-terminus or C-terminus of [D], or to a side chain of an amino acid constituting [D]. Furthermore, more than one [R] molecule can be linked to one molecule of [D]. In this case, the [R] molecules can be introduced to different positions on the [D] molecule. Alternatively, [D] can be modified with a number of [R]s linked together.

For example, there have been reported a variety of naturally-occurring or artificially synthesized polypeptides having cell-membrane permeability (Joliot A. & Prochiantz A., Nat Cell Biol. 2004; 6: 189-96). All of these known cell-membrane permeable substances can be used for modifying the mutant polypeptides in the present invention.

The membrane transducing agent can be selected from the group listed below:

[poly-arginine], Matsushita, M. et al, J Neurosci. 21, 6000-7 (2003);

[Tat/RKKRRQRRR] (SEQ ID NO: 6) Frankel, A. et al, Cell 55, 1189-93 (1988) and Green, M. & Loewenstein, P. M. Cell 55, 1179-88 (1988);

[Penetratin/RQIKIWFQNRRMKWKK] (SEQ ID NO: 7), Derossi, D. et al, J. Biol. Chem. 269, 10444-50 (1994);

[Buforin II/TRSSRAGLQFPVGRVHRLLRK] (SEQ ID NO: 8) Park, C. B. et al. Proc. Natl Acad. Sci. USA 97, 8245-50 (2000);

[Transportan/GWTLNSAGYLLGKINLKALAALAKKIL] (SEQ ID NO: 9) Pooga, M. et al. FASEB J. 12, 67-77 (1998);

[MAP (model amphipathic peptide)/KLALKLALKALKAALKLA] (SEQ ID NO: 10), Oehlke, J. et al. Biochim. Biophys. Acta. 1414, 127-39 (1998);

[K-FGF/AAVALLPAVLLALLAP] (SEQ ID NO: 11), Lin, Y. Z. et al. J. Biol. Chem. 270, 14255-14258 (1995);

[Ku70/VPMLK] (SEQ ID NO: 12), Sawada, M. et al. Nature Cell Biol. 5, 352-7 (2003);

[Ku70/PMLKE] (SEQ ID NO: 13), Sawada, M. et al. Nature Cell Biol. 5, 352-7 (2003);

[Prion/MANLGYWLLALFVTMWTDVGLCKKRPKP] (SEQ ID NO: 14), Lundberg, P. et al. Biochem. Biophys. Res. Commun. 299, 85-90 (2002);

[pVEC/LLIILRRRIRKQAHAHSK] (SEQ ID NO: 15), Elmquist, A. et al. Exp. Cell Res. 269, 237-44 (2001);

[Pep-1/KETWWETWWTEWSQPKKKRKV] (SEQ ID NO: 16), Morris, M. C. et al. Nature Biotechnol. 19, 1173-6 (2001);

[SynB1/RGGRLSYSRRRFSTSTGR] (SEQ ID NO: 17), Rousselle, C. et al. Mol. Pharmacol. 57, 679-86 (2000);

[Pep-7/SDLWEMMMVSLACQY] (SEQ ID NO: 18), Gao, C. et al. Bioorg. Med. Chem. 10, 4057-65 (2002); and

[HN-1/TSPLNIHNGQKL] (SEQ ID NO: 19), Hong, F. D. & Clayman, G. L. Cancer Res. 60, 6551-6 (2000).

In the present invention, the number of arginine residues that constitute the poly-arginine is not limited. In some preferred embodiments, 5 to 20 contiguous arginine residues may be exemplified. In a preferred embodiment, the number of arginine residues of the poly-arginine is 11 (SEQ ID NO: 22).

The present invention further provides an isolated polynucleotide encoding the dominant negative protein of the present invention and vectors and host cells including the polynucleotides. Polynucleotides, vectors and host cells are already defined above.

Treating and preventing lung or esophageal cancer with dominant negative mutants

The dominant negative mutants of CDCR5 disclosed herein can be used for treating or preventing lung or esophageal cancer. For example, the present invention provides methods for either or both treating and preventing lung or esophageal cancer in a subject by administering a CDCA5 mutant having a dominant negative effect, a polynucleotide encoding such a mutant, or a vector including the polynucleotide.

In some preferred embodiments, the CDCA5 mutant is linked to a membrane transducing agent for the administration to a subject. A number of peptide sequences have been characterized for their ability to translocate into live cells and can be used for this purpose in the present invention. Such membrane transducing agents (typically peptides) are defined by their ability to reach the cytoplasmic and/or nuclear compartments in live cells after internalization. Examples of proteins from which transducing agents may be derived include HIV Tat transactivator1, 2, the Drosophila melanogaster transcription factor Antennapedia3. In addition, nonnatural peptides with transducing activity have been used. These peptides are typically small peptides known for their membrane-interacting properties which are tested for translocation. The hydrophobic region within the secretion signal sequence of K-fibroblast growth factor (FGF), the venom toxin mastoparan (transportan)13, and Buforin I14 (an amphibian antimicrobial peptide) have been shown to be useful as transducing agents. For a review of transducing agents useful in the present invention see Joliot et al. Nature Cell Biology 6:189-96 (2004).

The CDCA5 mutant use for the treatment or prevention may preferably have the general formula:

[R]-[D], or

[D]-[R],

wherein [R] is a membrane transducing agent, and [D] is a polypeptide having the amino acid sequence of SEQ ID NO: 7 as defined above under the item of “Dominant negative protein that inhibits ERK kinase activity for CDCA5”.

In another embodiment, the present invention provides methods for treating or preventing lung or esophageal cancer in a subject, including the step of administering a vector expressing the CDCA5 mutant of the present invention. In this embodiment, the CDCA5 mutant may be introduced into cells without a membrane transducing agent, since such vector expresses the CDCA5 within the cells.

In order to express the vector in animal cells, such as CHO, COS or NIH3T3 cells, the vector should have a promoter necessary for expression in such cells, for example, the SV40 promoter (Mulligan et al., Nature 277: 108 (1979)), the MMLV-LTR promoter, the EF1 alpha promoter (Mizushima et al., Nucleic Acids Res 18: 5322 (1990)), the CMV promoter and the like, and preferably a marker gene for selecting transformants (for example, a drug resistance gene selected by a drug (e.g., neomycin, G418)). Examples of known vectors with these characteristics include, for example, pMAM, pDR2, pBK-RSV, pBK-CMV, pOPRSV and pOP13.

Pharmaceutical Compositions

The present invention further provides a composition for treating or preventing lung cancer or esophageal cancer, which is composed of a pharmaceutically effective amount of a compound that decreases a kinase activity of ERK for CDCA5 or inhibits the phosphorylation of CDCA5 by ERK, and a pharmaceutically acceptable carrier. Whether or not a subject compound has the target activity can be determined in accordance with, for example, the screening methods of the present invention. For example, the kinase activity for CDCA5 can be determined by incubating the subject compound under conditions suitable for phosphorylation of CDCA5 and detecting the phosphor-CDCA5 level. The phosphorylation site of CDCA5 by ERK can be Ser79 or Ser209.

Further, the dominant negative polypeptides of the present invention suppress the ERK-dependent phosphorylation on CDCA5. Therefore, according to another aspect of the present invention, the present pharmaceutical composition, composed of either the dominant negative polypeptide of the present invention or a polynucleotide encoding the polypeptide, can be used to inhibit the ERK-dependent phosphorylation on CDCA5. Moreover, the present inventors revealed that the ERK-dependent phosphorylation on CDCA5 is indispensable for lung cancer or esophageal cell growth and/or survival. Thus, the present pharmaceutical compositions, composed of either the polypeptide or the polynucleotide can be used to treat or prevent lung or esophageal cancer, in particular, NSCLC. That is, in one embodiment of the present invention, the present invention provides:

[1] A composition for treating or preventing lung or esophageal cancer, said composition including a pharmaceutically effective amount of a CDCA5 mutant having a dominant negative effect, a polynucleotide encoding said mutant, or a vector including the polynucleotide as an active ingredient, and a pharmaceutically acceptable carrier,

[2] the composition of claim [1], wherein the CDCA5 mutant includes an amino acid sequence in which at least one ERK-dependent phosphorylation site on CDCA5 is substituted with an amino acid residue other than that of the wild type,

[3] the composition of [2], wherein the ERK-dependent phosphorylation site is either or both Ser-79, and Ser-209,

[4] the composition of [3], wherein the CDCA5 mutant includes the amino acid sequence of SEQ ID NO: 7,

[5] the composition of [4], wherein the CDCA5 mutant has the general formula:

[R]-[D],

wherein [R] is a membrane transducing agent, and [D] is a polypeptide including the amino acid sequence of SEQ ID NO: 7,

[6] the composition of [5], wherein the membrane transducing agent is selected from group consisting of;

poly-arginine;
SEQ ID NO: 8
Tat/RKKRRQRRR/;
SEQ ID NO: 9
Penetratin/RQIKIWFQNRRMKWKK/;
SEQ ID NO: 10
Buforin II/TRSSRAGLQFPVGRVHRLLRK/;
SEQ ID NO: 11
Transportan/GWTLNSAGYLLGKINLKALAALAKKIL/;
SEQ ID NO: 12
MAP (model amphipathic peptide)/
KLALKLALKALKAALKLA/;
SEQ ID NO: 13
K-FGF/AAVALLPAVLLALLAP/;
SEQ ID NO: 14
Ku70/VPMLK/;
SEQ ID NO: 15
Ku70/PMLKE/;
SEQ ID NO: 16
Prion/MANLGYWLLALFVTMWTDVGLCKKRPKP/;
SEQ ID NO: 17
pVEC/LLIILRRRIRKQAHAHSK/;
SEQ ID NO: 18
Pep-1/KETWWETWWTEWSQPKKKRKV/;
SEQ ID NO: 19
SynB1/RGGRLSYSRRRFSTSTGR/;
SEQ ID NO: 20
Pep-7/SDLWEMMMVSLACQY/;
and
SEQ ID NO: 21
HN-1/TSPLNIHNGQKL/.

Since the present dominant negative polypeptides exert their functions to suppress the growth of lung or esophageal cancer cell, the polypeptides in the pharmaceutical composition are required to be introduced into the cells in order to be effective. Therefore, polypeptides suitable for the pharmaceutical composition include those that are modified so as to penetrate into the cells. Examples of such modifications include, but are not limited to, linking to a cell-permeable peptide, sticking on the surface of a micro particle (e.g., metal particles), and inclusion into a liposome as detailed above.

In another embodiment, the present invention also provides the use of the dominant negative polypeptide of the present invention or the compounds screened by the present invention in manufacturing a pharmaceutical composition for treating cancer, such as lung and esophageal cancer.

Alternatively, the present invention further provides the dominant negative polypeptide of the present invention or the compounds screened by the present invention for use in treating cancer, such as lung and esophageal cancer.

Alternatively, the present invention further provides a method or process for manufacturing a pharmaceutical composition for treating cancer, such as lung and esophageal cancer, wherein the method or process includes the step for formulating a pharmaceutically or physiologically acceptable carrier with the polypeptide or compound as active ingredients.

In another embodiment, the present invention also provides a method or process for manufacturing a pharmaceutical composition for treating cancer, such as lung and esophageal cancer, wherein the method or process includes step for admixing an active ingredient with a pharmaceutically or physiologically acceptable carrier, wherein the active ingredient is the dominant negative polypeptide of the present invention or the compounds screened by the present invention.

The pharmaceutical compositions of the present invention may also be used to treat and/or prevent disorders in human and any other mammal including, but not limited to, mouse, rat, guinea-pig, rabbit, cat, dog, sheep, goat, pig, cattle, horse, monkey, baboon, and chimpanzee, particularly a commercially important animal or a domesticated animal.

The pharmaceutical compositions of the present invention include the active ingredients (the dominant negative polypeptide of the present invention, a polynucleotide encoding the polypeptide, or a compound isolated by the screening methods of the present invention) at a pharmaceutically effective amount. A “pharmaceutically effective amount” of a compound (including proteins and polynucleotides) is a quantity that is sufficient to treat and/or prevent objective disorders wherein the ERK-dependent phosphorylation on CDCA5 plays important roles. An example of a pharmaceutically effective amount may be an amount that is needed to decrease the ERK-dependent phosphorylation level on CDCA5 when administered to a patient, so as to thereby treat or prevent the disorders. The decrease in the phosphorylation level may be, for example, at least a decrease of about 5%, 10%, 20%, 30%, 40%, 50%, 75%, 80%, 90%, 95%, 99%, or 100%. Alternatively, a pharmaceutically effective amount may be an amount that leads to a decrease in size, prevalence, or metastatic potential of lung or esophageal cancer in a subject. Furthermore, when the pharmaceutical composition of the present invention is applied prophylactically, the “pharmaceutically effective amount” may be an amount which retards or prevents occurrence of lung or esophageal cancer or alleviates a clinical symptom of lung or esophageal cancer.

The assessment of lung or esophageal cancer to determine such a pharmaceutically effective amount of a polypeptide or polynucleotide of the present invention can be made using standard clinical protocols, including histopathologic diagnosis or through identification of symptomatic anomalies such as chronic cough, hoarseness, coughing up blood, weight loss, loss of appetite, shortness of breath, wheezing, repeated bouts of bronchitis or pneumonia, and chest pain.

The dose employed will depend upon a number of factors, including the age and sex of the subject, the precise disorder being treated, and its severity. In addition, the route of administration may vary depending upon the condition and its severity. However, the determination of an effective dose range for the medicaments of the present invention is well within the capability of those skilled in the art, especially in light of the detailed disclosure provide herein. The pharmaceutically or preventively effective amount (dose) of the dominant negative polypeptide of the present invention, a polynucleotide encoding the polypeptide, or a compound isolated by the screening methods of the present invention can be estimated initially from cell culture assays and/or animal models. For example, a dose can be formulated in animal models to achieve a circulating concentration range that includes the IC₅₀ (the dose where 50% of the cells show the desired effects) as determined in cell culture. Toxicity and therapeutic efficacy of the polypeptide, polynucleotide or compound also can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (i.e., the ratio between LD₅₀ and ED₅₀). Polypeptides, polynucleotides and compounds which exhibit high therapeutic indices are preferable. The data obtained from these cell culture assays and animal studies may be used in formulating a dosage range for use in humans. The dosage of such polypeptides and polynucleotides may lie within a range of circulating concentrations that include the ED₅₀ with little or no toxicity. The dosage may vary within this range depending upon the dosage form employed and the route of administration utilized. The exact formulation, route of administration and dosage can be chosen by an individual physician in view of a patient's condition (see, e.g., Fingl et al. (1975) in “The Pharmacological Basis of Therapeutics”, Ch. 1 p 1). Dosage amount and interval may be adjusted individually to provide plasma levels of the active ingredient sufficient to maintain the desired effect.

If needed, the pharmaceutical compositions of the present invention, composed of the dominant negative polypeptide of the present invention, a polynucleotide encoding the polypeptide, or a compound isolated by the present screening methods may optionally include other therapeutic substances as an active ingredient, so long as the substance does not inhibit the in vivo dominant negative effect of the polypeptide of interest. For example, formulations may include anti-inflammatory agents, pain killers, chemotherapeutics, and the like. In addition to including other therapeutic substances in the medicament itself, the medicaments of the present invention may also be administered sequentially or concurrently with the one or more other pharmacologic agents. The amounts of medicament and pharmacologic agent depend, for example, on what type of pharmacologic agent(s) is/are used, the disease being treated, and the scheduling and routes of administration.

It should be understood that in addition to the ingredients particularly mentioned herein, the formulations of this invention may include other agents conventional in the art having regard to the type of formulation in question.

In one embodiment of the present invention, the present pharmaceutical compositions may be included in articles of manufacture and kits containing materials useful for treating the pathological conditions of lung or esophageal cancer, particularly NSCLC. The article of manufacture may include a container of any of the present pharmaceutical compositions with a label. Suitable containers include bottles, vials, and test tubes. The containers may be formed from a variety of materials, such as glass or plastic. The label on the container should indicate the composition is used for treating or preventing one or more conditions of the disease. The label may also indicate directions for administration and so on.

In addition to the container described above, a kit including a pharmaceutical composition of the present invention may optionally further include a second container housing a pharmaceutically-acceptable diluent. It may further include other materials desirable from a commercial and user standpoint, including other buffers, diluents, filters, needles, syringes, and package inserts with instructions for use.

The pharmaceutical compositions may, if desired, be presented in a pack or dispenser device which may contain one or more unit dosage forms containing the active ingredient. The pack may, for example, include metal or plastic foil, such as a blister pack. The pack or dispenser device may be accompanied by instructions for administration.

(1) Pharmaceutical Compositions Containing a Polypeptide as an Active Ingredient:

The present invention provides a pharmaceutical composition which contains as an active ingredient the dominant negative polypeptide of the present invention. Since the present polypeptides exert their functions to suppress the proliferation of CDCA5 expressing cells, the polypeptides in the pharmaceutical composition are required to be introduced into the cells in order to be effective. Therefore, polypeptides suitable for the pharmaceutical composition include those that are modified so as to penetrate into the cells. Examples of such modifications include, but are not limited to, linking to a cell-permeable peptide, sticking on the surface of a micro particle (e.g., metal particles), and inclusion into a liposome. The use of a cell-permeable peptide is particularly preferred for the present pharmaceutical compounds.

For the treatment and/or prevention of cancer, such as lung and esophageal cancer, the dominant negative polypeptides of the present invention may be directly administered as a pharmaceutical composition to the patient or may be formulated according to conventional formulation methods. For example, if needed, the polypeptides may be formulated into a form suitable for oral, rectal, nasal, topical (including buccal and sub-lingual), vaginal or parenteral (including intramuscular, subcutaneous, intravenous, intratumoral) administration, or for administration by inhalation or insufflation. Thus, the present invention encompasses pharmaceutical compositions which include any pharmaceutically acceptable excipient or carrier in addition to the polypeptide. The phrase “pharmaceutically acceptable” indicates that the substance is inert and includes conventional substances used as diluent or vehicle for a drug. Suitable excipients and their formulations are described, for example, in Remington's Pharmaceutical Sciences, 16^{th} ed. (1980) Mack Publishing Co., ed. Oslo et al.

Excipients may be used, for example, to maintain the correct pH of the formulation. For optimal shelf life, the pH of solutions containing a polypeptide is preferably from about 5 to about 8, and more preferably form about 7 to about 7.5. The formulation may also include a lyophilized powder or other optional excipients suitable for the present invention.

For aqueous preparations, an appropriate amount of a pharmaceutically-acceptable salt is typically used in the formulation to render the formulation isotonic. Examples of the pharmaceutically-acceptable isotonic excipients include, but are not limited to, liquids such as saline, Ringer's solution, Hanks's solution, and dextrose solution. Isotonic excipients are particularly important for injectable formulations.

Pharmaceutical formulations suitable for oral administration include, but are not limited to, capsules, cachets, and tablets, each containing a predetermined amount of the active ingredient. Formulations also include drags, liquids, gels, syrups, slurries, pills, powders, granules, solutions, suspension, emulsions, and the like. The active ingredient is optionally administered as a bolus electuary or paste. Tablets and capsules for oral administration may contain conventional excipients, such as binding agents, fillers, lubricants, disintegrants, and wetting agents. Suitable excipients are, in particular, fillers such as sugars, including lactose, sucrose, mannitol, and sorbitol; cellulose preparations such as, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents may be added, such as the cross-linked polyvinyl pyrrolidone, agar, and alginic acid or a salt thereof such as sodium alginate. A tablet may be made by compression or molding, optionally with one or more formulational ingredients. Compressed tablets may be prepared by compressing in a suitable machine the active ingredient in a free-flowing form such as powder or granules, optionally mixed with a binder, lubricant, inert diluent, lubricating surface active or dispersing agent. Molded tablets may be made via molding in a suitable machine a mixture of the powdered compound moistened with an inert liquid diluent. The tablets may be coated according to methods well known in the art. Oral fluid preparations may be in the form of, for example, aqueous or oily suspensions, solutions, emulsions, syrups, or elixirs, or may be presented as a dry product for reconstitution with water or other suitable vehicle prior to use. Such liquid preparations may contain conventional additives such as suspending agents, emulsifying agents, non-aqueous vehicles (which may include edible oils), and preservatives. The formulation or dose of medicament in these preparations makes a suitable dosage within the indicated range acquirable.

Although dosages may vary according to the symptoms, an exemplary dose of the polypeptide or fragments thereof for treating or preventing lung or esophageal cancer is about 0.1 mg to about 100 mg per day, preferably about 1.0 mg to about 50 mg per day and more preferably about 1.0 mg to about 20 mg per day, when administered orally to a normal adult (weight 60 kg).

When administering parenterally, in the form of an injection to a normal adult (weight 60 kg), although there are some differences according to the condition of the patient, symptoms of the disease and method of administration, it is convenient to intravenously inject a dose of about 0.01 mg to about 30 mg per day, preferably about 0.1 to about 20 mg per day and more preferably about 0.1 to about 10 mg per day. Also, in the case of other animals too, it is possible to administer an amount converted to 60 kg of body-weight.

Formulations for parenteral administration include aqueous and non-aqueous sterile injection solutions which may additionally contain anti-oxidants, buffers, bacteriostats, and solutes which render the formulation isotonic with the blood of the intended recipient; likewise, aqueous and non-aqueous sterile suspensions which may include suspending agents and thickening agents. The formulations may be presented in unit dose or multi-dose containers, for example, sealed ampoules and vials, and may be stored in a freeze-dried (lyophilized) condition requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules, and tablets of the kind previously described.

Formulations for rectal administration include suppositories with standard carriers, such as cocoa butter or polyethylene glycol. Formulation for topical administration in the mouth, for example, buccally or sublingually, include lozenges, which contain the active ingredient in a flavored base such as sucrose and acacia or tragacanth, and pastilles containing the active ingredient in a base such as gelatin, glycerin, sucrose, or acacia. For intra-nasal administration of an active ingredient, a liquid spray or dispersible powder or in the form of drops may be used. Drops may be formulated with an aqueous or non-aqueous base also containing one or more dispersing agents, solubilizing agents, or suspending agents.

For administration by inhalation the composition is conveniently delivered from an insufflator, nebulizer, pressurized packs, or other convenient means of delivering an aerosol spray. Pressurized packs typically include a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Alternatively, for administration by inhalation or insufflation, the compositions may take the form of a dry powder composition, for example, a powder mix of an active ingredient and a suitable powder base such as lactose or starch. The powder composition may be presented in unit dosage form in, for example, capsules, cartridges, gelatin, or blister packs from which the powder may be administered with the aid of an inhalator or insufflators.

Other formulations include implantable devices and adhesive patches which release the therapeutic agent.

When desired, the above-described formulations, adapted to give slow, controlled, or sustained in vivo release of the active ingredient, may be employed.

(2) Pharmaceutical Compositions Containing a Polynucleotide as an Active Ingredient:

In addition, the present invention provides a pharmaceutical composition which contains, as an active ingredient, a polynucleotide encoding the dominant negative polypeptide of the present invention in an expressible form. Herein, the phrase “in an expressible form” means that the polynucleotide, when introduced into a cell, will be expressed in vivo as a polypeptide that has dominant negative effect. In a preferred embodiment, the nucleic acid sequence of the polynucleotide of interest includes regulatory elements necessary for expression of the polynucleotide in a target cell. The polynucleotide may be equipped to be stably inserted into the genome of the target cell (see, e.g., Thomas K. R. & Capecchi M. R. (1987) Cell 51: 503-12. for a description of homologous recombination cassette vectors).

Delivery of a polynucleotide into a patient may be either direct, in which case the patient is directly exposed to a polynucleotide-carrying vector, or indirect, in which case, cells are first transformed with the polynucleotide of interest in vitro, and then transplanted into the patient. Theses two approaches are known, respectively, as in vivo or ex vivo gene therapy.

For general reviews of the methods of gene therapy, see Goldspiel et al., (1993) Clinical Pharmacy 12: 488-505; Wu and Wu (1991) Biotherapy 3: 87-95; Tolstoshev (1993) Ann. Rev. Pharmacol. Toxicol. 33: 573-96; Mulligan (1993) Science 260: 926-32; Morgan & Anderson (1993) Ann. Rev. Biochem. 62: 191-217; and (1993) Trends in Biotechnology 11(5): 155-215. Methods commonly known in the art of recombinant DNA technology which can be used are described in eds. Ausubel et al. (1993) Current Protocols in Molecular Biology, John Wiley & Sons, NY; and Krieger (1990) Gene Transfer and Expression, A Laboratory Manual, Stockton Press, NY.

(3) Pharmaceutical Compositions Containing Compounds Selected By the Screening Methods of the Invention

The present invention provides compositions for treating or preventing lung cancer or esophageal cancer containing any of the compounds selected by the screening methods of the present invention.

When administrating a compound isolated by a method of the present invention as a pharmaceutical for humans and other mammals, such as mice, rats, guinea-pigs, rabbits, cats, dogs, sheep, pigs, cattle, monkeys, baboons, and chimpanzees, the isolated compound can be directly administered or, alternatively, can be formulated into a dosage form using conventional pharmaceutical preparation methods. For example, according to the need, the drugs can be taken orally, as sugar-coated tablets, capsules, elixirs and microcapsules, or non-orally, in the form of injections of sterile solutions or suspensions with water or any other pharmaceutically acceptable liquid. For example, the compound can be mixed with pharmaceutically acceptable carriers or media, specifically, sterilized water, physiological saline, plant-oils, emulsifiers, suspending agents, surfactants, stabilizers, flavoring agents, excipients, vehicles, preservatives, binders, and such, in a unit dose form required for generally accepted drug implementation. The amount of active ingredients in these preparations makes a suitable dosage within the indicated range acquirable.

Examples of additives that can be mixed to form tablets and capsules include, for example, binders, such as gelatin, corn starch, tragacanth gum and arabic gum; excipients, such as crystalline cellulose; swelling agents, such as corn starch, gelatin and alginic acid; lubricants, such as magnesium stearate; sweeteners, such as sucrose, lactose or saccharin; and flavoring agents, such as peppermint, Gaultheria adenothrix oil and cherry. When the unit-dose form is a capsule, a liquid carrier, such as an oil, can also be further included in the above ingredients. Sterile composites for injections can be formulated following normal drug implementations using vehicles such as distilled water used for injections.

Physiological saline, glucose, and other isotonic liquids, including adjuvants, such as D-sorbitol, D-mannose, D-mannitol, and sodium chloride, can be used as aqueous solutions for injections. These can be used in conjunction with suitable solubilizers, such as alcohol, specifically ethanol, polyalcohols such as propylene glycol and polyethylene glycol, non-ionic surfactants, such as Polysorbate 80 (TM) and HCO-50. Sesame oil and soy-bean oil are examples of suitable oleaginous liquids and may be used in conjunction with benzyl benzoate or benzyl alcohol as solubilizers. They may be further formulated with a buffer, such as phosphate buffer or sodium acetate buffer; a pain-killer, such as procaine hydrochloride; a stabilizer, such as benzyl alcohol or phenol; and an anti-oxidant. The prepared injection may be filled into a suitable ampule.

Methods well known to those skilled in the art may be used to administer a pharmaceutical composition of the present invention to patients, for example, as intra-arterial, intravenous, or percutaneous injections and also as intranasal, transbronchial, intramuscular or oral administrations. The dosage and method of administration may vary according to the body-weight and age of the patient and the selected administration method; however, one skilled in the art can routinely select a suitable method of administration and dosage. If said compound is encodable by a DNA, the DNA can be inserted into a vector for gene therapy and the vector can be administered to a patient to perform the therapy. The dosage and method of administration may again vary according to the body-weight, age, and symptoms of the patient; however, one skilled in the art can suitably select them.

For example, although the dose of a compound that binds to ERK and regulates its activity or that inhibits the phosphorylation of CDCA5 depends on the symptoms, a suitable dose is generally about 0.1 mg to about 100 mg per day, preferably about 1.0 mg to about 50 mg per day and more preferably about 1.0 mg to about 20 mg per day, when administered orally to a normal adult (weight 60 kg).

When administering parenterally, in the form of an injection to a normal adult (weight 60 kg), although there are some differences according to the patient, target organ, symptoms and method of administration, it is convenient to intravenously inject a dose of about 0.01 mg to about 30 mg per day, preferably about 0.1 to about 20 mg per day and more preferably about 0.1 to about 10 mg per day. Also, in the case of other animals too, it is possible to administer an amount converted to 60 kg of body-weight.

The present invention includes pharmaceutical, or therapeutic, compositions containing one or more therapeutic compounds described herein. Pharmaceutical formulations may include those suitable for oral, rectal, nasal, topical (including buccal and sub-lingual), vaginal or parenteral (including intramuscular, subcutaneous and intravenous) administration, or for administration by inhalation or insufflation. The formulations may, where appropriate, be conveniently presented in discrete dosage units and may be prepared by any of the methods conventional in the art of pharmacy. All such Pharmaceutical methods herein include the steps of bringing into association the active compound with liquid carriers or finely divided solid carriers or both as needed and then, if necessary, shaping the product into the desired formulation.

Pharmaceutical formulations suitable for oral administration may conveniently be presented as discrete units, such as capsules, cachets or tablets, each containing a pre-determined amount of the active ingredient; as a powder or granules; or as a solution, a suspension or as an emulsion. The active ingredient may also be presented as a bolus electuary or paste, and be in a pure form, i.e., without a carrier. Tablets and capsules for oral administration may contain conventional excipients, such as binding agents, fillers, lubricants, disintegrant or wetting agents. A tablet may be made by compression or molding, optionally with one or more formulational ingredients. Compressed tablets may be prepared by compressing in a suitable machine the active ingredients in a free-flowing form, such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may be coated according to methods well known in the art. Oral fluid preparations may be in the form of, for example, aqueous or oily suspensions, solutions, emulsions, syrups or elixirs, or may be presented as a dry product for constitution with water or other suitable vehicle before use. Such liquid preparations may contain conventional additives, such as suspending agents, emulsifying agents, non-aqueous vehicles (which may include edible oils), or preservatives. Furthermore, the tablets may optionally be formulated so as to provide slow or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit dose or multi-dose containers, for example, sealed ampoules and vials, and may be stored in a freeze-dried (lyophilized) condition requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

Formulations for rectal administration may be presented as a suppository with the usual carriers, such as cocoa butter or polyethylene glycol. Formulations for topical administration in the mouth, for example, buccally or sublingually, include lozenges, containing the active ingredient in a flavored base, such as sucrose and acacia or tragacanth, and pastilles containing the active ingredient in a base, such as gelatin and glycerin or sucrose and acacia. For intra-nasal administration, the compounds of the present invention may be used as a liquid spray or dispersible powder or in the form of drops. Drops may be formulated with an aqueous or non-aqueous base also including one or more dispersing agents, solubilizing agents or suspending agents. Liquid sprays are conveniently delivered from pressurized packs.

For administration by inhalation, the compounds of the present invention are conveniently delivered from an insufflator, nebulizer, pressurized pack or other convenient aerosol spray delivery means. Pressurized packs may include a suitable propellant, such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds of the present invention may take the form of a dry powder composition, for example a powder mix of the compound and a suitable powder base, such as lactose or starch. The powder composition may be presented in a unit dosage form, in for example, capsules, cartridges, gelatin or blister packs from which the powder may be administered with the aid of an inhalator or insufflators.

When desired, the above-described formulations, adapted to give sustained release of the active ingredient, may be employed. The pharmaceutical compositions of the present invention may also contain other active ingredients, such as antimicrobial agents, immunosuppressants or preservatives.

It should be understood that in addition to the ingredients particularly mentioned above, the formulations of the present invention may include other agents conventional in the art, having regard to the type of formulation in question; for example, those suitable for oral administration may include flavoring agents.

Preferred unit dosage formulations are those containing an effective dose, as recited below, or an appropriate fraction thereof, of the active ingredient.

For each of the aforementioned conditions, the compositions may be administered orally or via injection at a dose ranging from about 0.1 to about 250 mg/kg per day. The dose range for adult humans is generally from about 5 mg to about 17.5 g/day, preferably about 5 mg to about 10 g/day, and most preferably about 100 mg to about 3 g/day. Tablets or other unit dosage forms of presentation provided in discrete units may conveniently contain an amount which is effective at such dosage or as a multiple of the same, for instance, units containing about 5 mg to about 500 mg, usually from about 100 mg to about 500 mg.

The pharmaceutical composition preferably is administered orally or by injection (intravenous or subcutaneous), and the precise amount administered to a subject will be the responsibility of the attendant physician. However, the dose employed will depend upon a number of factors, including the age and sex of the subject, the precise disorder being treated, and its severity. In addition, the route of administration may vary depending upon the condition and its severity.

Method for Assessing the Prognosis of Cancer:

The present invention relates to the novel discovery that CDCA5 expression is significantly associated with poorer prognosis of patients. Thus, the present invention provides a method for determining or assessing the prognosis of a patient with cancer, in particular lung cancer, by detecting the expression level of the CDCA5 gene in a biological sample of the patient; comparing the detected expression level to a control level; and determining an increased expression level to the control level as indicative of poor prognosis (poor survival).

Herein, the term “prognosis” refers to a forecast as to the probable outcome of the disease as well as the prospect of recovery from the disease as indicated by the nature and symptoms of the case. Accordingly, a less favorable, negative, poor prognosis is defined by a lower post-treatment survival term or survival rate. Conversely, a positive, favorable, or good prognosis is defined by an elevated post-treatment survival term or survival rate.

The terms “assessing the prognosis” refer to the ability of predicting, forecasting or correlating a given detection or measurement with a future outcome of cancer of the patient (e.g., malignancy, likelihood of curing cancer, survival, and the like). For example, a determination of the expression level of CDCA5 over time enables a predicting of an outcome for the patient (e.g., increase or decrease in malignancy, increase or decrease in grade of a cancer, likelihood of curing cancer, survival, and the like).

In the context of the present invention, the phrase “assessing (or determining) the prognosis” is intended to encompass predictions and likelihood analysis of cancer, progression, particularly cancer recurrence, metastatic spread and disease relapse. The present method for assessing prognosis is intended to be used clinically in making decisions concerning treatment modalities, including therapeutic intervention, diagnostic criteria such as disease staging, and disease monitoring and surveillance for metastasis or recurrence of neoplastic disease.

The patient-derived biological sample used for the method may be any sample derived from the subject to be assessed so long as the CDCA5 gene can be detected in the sample. Preferably, the biological sample is a lung cell (a cell obtained from the lung). Furthermore, the biological sample may include bodily fluids such as sputum, blood, serum, or plasma. Moreover, the sample may be cells purified from a tissue. The biological samples may be obtained from a patient at various time points, including before, during, and/or after a treatment.

According to the present invention, it was shown that the higher the expression level of the CDCA5 gene measured in the patient-derived biological sample, the poorer the prognosis for post-treatment remission, recovery, and/or survival and the higher the likelihood of poor clinical outcome. Thus, according to the present method, the “control level” used for comparison may be, for example, the expression level of the CDCA5 gene detected before any kind of treatment in an individual or a population of individuals who showed good or positive prognosis of cancer, after the treatment, which herein will be referred to as “good prognosis control level”. Alternatively, the “control level” may be the expression level of the CDCA5 gene detected before any kind of treatment in an individual or a population of individuals who showed poor or negative prognosis of cancer, after the treatment, which herein will be referred to as “poor prognosis control level”. The “control level” is a single expression pattern derived from a single reference population or from a plurality of expression patterns. Thus, the control level may be determined based on the expression level of the CDCA5 gene detected before any kind of treatment in a patient of cancer, or a population of the patients whose disease state (good or poor prognosis) is known. Preferably, cancer is lung cancer. It is preferred, to use the standard value of the expression levels of the CDCA5 gene in a patient group with a known disease state. The standard value may be obtained by any method known in the art. For example, a range of mean +/−2 S.D. or mean +/−3 S.D. may be used as standard value.

The control level may be determined at the same time with the test biological sample by using a sample(s) previously collected and stored before any kind of treatment from cancer patient(s) (control or control group) whose disease state (good prognosis or poor prognosis) are known.

Alternatively, the control level may be determined by a statistical method based on the results obtained by analyzing the expression level of the CDCA5 gene in samples previously collected and stored from a control group. Furthermore, the control level can be a database of expression patterns from previously tested cells.

Moreover, according to an aspect of the present invention, the expression level of the CDCA5 gene in a biological sample may be compared to multiple control levels, which control levels are determined from multiple reference samples. It is preferred to use a control level determined from a reference sample derived from a tissue type similar to that of the patient-derived biological sample.

According to the present invention, a similarity in the expression level of the CDCA5 gene to a good prognosis control level indicates a more favorable prognosis of the patient and an increase in the expression level to the good prognosis control level indicates less favorable, poorer prognosis for post-treatment remission, recovery, survival, and/or clinical outcome. On the other hand, a decrease in the expression level of the CDCA5 gene to the poor prognosis control level indicates a more favorable prognosis of the patient and a similarity in the expression level to the poor prognosis control level indicates less favorable, poorer prognosis for post-treatment remission, recovery, survival, and/or clinical outcome.

The expression level of the CDCA5 gene in a biological sample can be considered altered when the expression level differs from the control level by more than 1.0, 1.5, 2.0, 5.0, 10.0, or more fold.

The difference in the expression level between the test biological sample and the control level can be normalized to a control, e.g., housekeeping gene. For example, polynucleotides whose expression levels are known not to differ between the cancerous and non-cancerous cells, including those coding for beta-actin, glyceraldehyde 3-phosphate dehydrogenase, and ribosomal protein P1, may be used to normalize the expression levels of the CDCA5 genes.

The expression level may be determined by detecting the gene transcript in the patient-derived biological sample using techniques well known in the art. The gene transcripts detected by the present method include both the transcription and translation products, such as mRNA and protein.

For instance, the transcription product of the CDCA5 gene can be detected by hybridization, e.g., Northern blot hybridization analyses, that use a CDCA5 gene probe to the gene transcript. The detection may be carried out on a chip or an array. The use of an array is preferable for detecting the expression level of a plurality of genes including the CDCA5 gene. As another example, amplification-based detection methods, such as reverse-transcription based polymerase chain reaction (RT-PCR) which use primers specific to the CDCA5 gene may be employed for the detection (see Example). The CDCA5 gene-specific probe or primers may be designed and prepared using conventional techniques by referring to the whole sequence of the CDCA5 gene (SEQ ID NO: 4). For example, the primers (SEQ ID NOs: 25-28) used in the Example may be employed for the detection by RT-PCR, but the present invention is not restricted thereto.

Specifically, a probe or primer used for the present method hybridizes under stringent, moderately stringent, or low stringent conditions to the mRNA of the CDCA5 gene. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5 degrees C. lower than the thermal melting point (Tm) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degree Centigrade for short probes or primers (e.g., 10 to 50 nucleotides) and at least about 60 degree Centigrade for longer probes or primers. Stringent conditions may also be achieved with the addition of destabilizing agents, such as formamide.

Alternatively, the translation product may be detected for the assessment of the present invention. For example, the quantity of the CDCA5 protein may be determined. A method for determining the quantity of the protein as the translation product includes immunoassay methods that use an antibody specifically recognizing the CDCA5 protein. The antibody may be monoclonal or polyclonal. Furthermore, any fragment or modification (e.g., chimeric antibody, scFv, Fab, F(ab′)2, Fv, etc.) of the antibody may be used for the detection, so long as the fragment retains the binding ability to the CDCA5 protein. Methods to prepare these kinds of antibodies for the detection of proteins are well known in the art, and any method may be employed in the present invention to prepare such antibodies and equivalents thereof.

As another method to detect the expression level of the CDCA5 gene based on its translation product, the intensity of staining may be observed via immunohistochemical analysis using an antibody against CDCA5 protein. Namely, the observation of strong staining indicates increased presence of the CDCA5 protein and at the same time high expression level of the CDCA5 gene.

Furthermore, the CDCA5 protein is known to have a cell proliferating activity. Therefore, the expression level of the CDCA5 gene can be determined using such cell proliferating activity as an index. For example, cells which express CDCA5 are prepared and cultured in the presence of a biological sample, and then by detecting the speed of proliferation, or by measuring the cell cycle or the colony forming ability the cell proliferating activity of the biological sample can be determined.

Moreover, in addition to the expression level of the CDCA5 gene, the expression level of other lung cancer-associated genes, for example, genes known to be differentially expressed in lung cancer may also be determined to improve the accuracy of the assessment. Examples of such other lung cell-associated genes include those described in WO 2004/031413 and WO 2005/090603, the contents of which are incorporated by reference herein.

Alternatively, according to the present invention, an intermediate result may also be provided in addition to other test results for assessing the prognosis of a subject. Such intermediate result may assist a doctor, nurse, or other practitioner to assess, determine, or estimate the prognosis of a subject. Additional information that may be considered, in combination with the intermediate result obtained by the present invention, to assess prognosis includes clinical symptoms and physical conditions of a subject.

The patient to be assessed for the prognosis of cancer according to the method is preferably a mammal and includes human, non-human primate, mouse, rat, dog, cat, horse, and cow.

A Kit for Diagnosing Cancer or Assessing the Prognosis of Cancer:

The present invention provides a kit for diagnosing cancer or assessing the prognosis of cancer. Preferably, the cancer is lung cancer. Specifically, the kit includes at least one reagent for detecting the expression of the CDCA5 gene in a patient-derived biological sample, which reagent may be selected from the group of:

(a) a reagent for detecting mRNA of the CDCA5 gene;

(b) a reagent for detecting the CDCA5 protein; and

(c) a reagent for detecting the biological activity of the CDCA5 protein.

Suitable reagents for detecting mRNA of the CDCA5 gene include nucleic acids that specifically bind to or identify the CDCA5 mRNA, such as oligonucleotides which have a complementary sequence to a part of the CDCA5 mRNA. These kinds of oligonucleotides are exemplified by primers and probes that are specific to the CDCA5 mRNA. These kinds of oligonucleotides may be prepared based on methods well known in the art. If needed, the reagent for detecting the CDCA5 mRNA may be immobilized on a solid matrix. Moreover, more than one reagent for detecting the CDCA5 mRNA may be included in the kit.

On the other hand, suitable reagents for detecting the CDCA5 protein include antibodies to the CDCA5 protein. The antibody may be monoclonal or polyclonal. Furthermore, any fragment or modification (e.g., chimeric antibody, scFv, Fab, F(ab′)2, Fv, etc.) of the antibody may be used as the reagent, so long as the fragment retains the binding ability to the CDCA5 protein. Methods to prepare these kinds of antibodies for the detection of proteins are well known in the art, and any method may be employed in the present invention to prepare such antibodies and equivalents thereof. Furthermore, the antibody may be labeled with signal generating molecules via direct linkage or an indirect labeling technique. Labels and methods for labeling antibodies and detecting the binding of antibodies to their targets are well known in the art and any labels and methods may be employed for the present invention. Moreover, more than one reagent for detecting the CDCA5 protein may be included in the kit.

Furthermore, the biological activity can be determined by, for example, measuring the cell proliferating activity due to the expressed CDCA5 protein in the biological sample. For example, the cell is cultured in the presence of a patient-derived biological sample, and then by detecting the speed of proliferation, or by measuring the cell cycle or the colony forming ability the cell proliferating activity of the biological sample can be determined. If needed, the reagent for detecting the CDCA5 mRNA may be immobilized on a solid matrix. Moreover, more than one reagent for detecting the biological activity of the CDCA5 protein may be included in the kit.

The kit may contain more than one of the aforementioned reagents. Furthermore, the kit may include a solid matrix and reagent for binding a probe against the CDCA5 gene or antibody against the CDCA5 protein, a medium and container for culturing cells, positive and negative control reagents, and a secondary antibody for detecting an antibody against the CDCA5 protein. For example, tissue samples obtained from patient with good prognosis or poor prognosis may serve as useful control reagents. A kit of the present invention may further include other materials desirable from a commercial and user standpoint, including buffers, diluents, filters, needles, syringes, and package inserts (e.g., written, tape, CD-ROM, etc.) with instructions for use. These reagents and such may be comprised in a container with a label. Suitable containers include bottles, vials, and test tubes. The containers may be formed from a variety of materials, such as glass or plastic.

As an embodiment of the present invention, when the reagent is a probe against the CDCA5 mRNA, the reagent may be immobilized on a solid matrix, such as a porous strip, to form at least one detection site. The measurement or detection region of the porous strip may include a plurality of sites, each containing a nucleic acid (probe). A test strip may also contain sites for negative and/or positive controls. Alternatively, control sites may be located on a strip separated from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of CDCA5 mRNA present in the sample. The detection sites may be configured in any suitably detectable shape and are typically in the shape of a bar or dot spanning the width of a test strip.

The kit of the present invention may further include a positive control sample or CDCA5 standard sample. The positive control sample of the present invention may be prepared by collecting CDCA5 positive blood samples and then those CDCA5 level are assayed. Alternatively, purified CDCA5 protein or polynucleotide may be added to CDCA5 free serum to form the positive sample or the CDCA5 standard. In the present invention, purified KDD 1 may be recombinant protein. The CDCA5 level of the positive control sample is, for example more than cut off value.

### EXAMPLES

Materials and Methods

Cell lines and clinical samples. The 23 human lung-cancer cell lines used for this study included nineteen NSCLCs (A427, A549, NCI-H1373, LC319, PC-14, PC-3, PC-9, NCI-H1666, NCI-H1781, NCI-H647, NCI-H226, NCI-H1703, NCI-H520, LU61, RERF-LC-AI, SK-MES-1, EBC-1, LX1, and NCI-H2170) and four small-cell lung cancers (SCLC: DMS114, DMS273, SBC-3, and SBC-5). The human esophageal carcinoma cell lines used in this study were as follows: nine SCC cell lines (TE1, TE2, TE3, TE4, TE5, TE6, TE8, TE9, and TE10) and one adenocarcinoma (ADC) cell line (TE7) (Nishihira T et al. J Cancer Res Clin Oncol 1993; 119: 441-49). All cells were grown in monolayers in appropriate media supplemented with 10% fetal calf serum (FCS) and were maintained at 37 degrees C. in an atmosphere of humidified air with 5% CO₂. Human airway epithelial cells, SAEC (Cambrex Bio Science Inc., East Rutherford, N.J.) was also included in the panel of the cells used in this study. Primary NSCLC and ESCC samples had been obtained earlier as reported elsewhere. All tumors were staged on the basis of the pTNM pathological classification of the UICC (International Union Against Cancer) (Sobin L, Wittekind C. Anonymous. New York: Wiley-Liss. 2002). A total of 262 NSCLCs (156 adenocarcinomas (ADCs), 88 squamous-cell carcinomas (SCCs), 2 adenosquamous carcinomas (ASCs), 16 large-cell carcinomas (LCCs); 88 female and 174 male patients; median age of 65.0 with a range of 26 to 84 years; 111 pT1, 125 pT2, 26 pT3 tumor size; 204 pN0, 24 pN1, 34 pN2 node status) and adjacent normal lung-tissue samples for immunostaining on formalin-fixed tissue microarray were also obtained from patients who had undergone surgery at Hokkaido University and its affiliated hospitals (Sapporo, Japan). This study and the use of all clinical materials mentioned were approved by individual institutional Ethical Committees.

Semiquantitative RT-PCR.

Appropriate dilutions of each single-stranded cDNA were prepared from mRNAs of clinical lung and esophageal cancer samples, taking the level of beta-actin (ACTB) expression as a quantitative control. The primer sets for amplification were as follows: ACTB-F (5′-GAGGTGATAGCATTGCTTTCG-3′; SEQ ID NO: 23) and ACTB-R (5′-CAAGTCAGTGTACAGGTAAGC-3′; SEQ ID NO: 24) for ACTB, CDCA5-F (5′-CGCCAGAGACTTGGAAATGT-3′; SEQ ID NO: 25) and CDCA5-R (5′-GTTTCTGTTTCTCGGGTGGT-3′; SEQ ID NO: 26) for CDCA5. All reactions involved initial denaturation at 95 degrees C. for 5 min followed by 22 (for ACTB) or 30 (for CDCA5) cycles of 95 degrees C. for 30 s, 56 degrees C. for 30 s, and 72 degrees C. for 60 s on a GeneAmp PCR system 9700 (Applied Biosystems, Foster City, Calif.).

Immunocytochemical Analysis.

COS-7 cells were transiently transfected with c-Myc-tagged CDCA5 (pcDNA3.1/myc-His-CDCA5) on glass coverslips (Becton Dickinson Labware, Franklin Lakes, N.J.). After 48 hours, the cells were fixed with 4% paraformaldehyde and then rendered permeable with PBS (−) containing 0.1% Triton X-100 for 3 min at room temperature. Nonspecific binding was blocked by Casblock (ZYMED, San Francisco, Calif.) for 10 min at room temperature. The cells were then incubated for 60 min at room temperature with primary antibodies diluted in PBS containing 3% BSA (rabbit polyclonal anti-c-Myc antibody, Santa Cruz Biotechnology, Santa Cruz, Calif.). After being washed with PBS, the cells were stained by Alexa 488-conjugated anti-rabbit secondary antibody (Molecular Probes) at 1:1,000 dilutions for 60 min at room temperature. After another wash with PBS (−), each specimen was mounted with Vectashield (Vector Laboratories, Inc., Burlingame, Calif.) containing 4′,6-diamidino-2-phenylindole and visualized with Spectral Confocal Scanning Systems (TSC SP2 AOBS; Leica Microsystems, Wetzlar, Germany).

Northern-Blot Analysis.

Human multiple-tissue blots (23 normal tissues including heart, brain, placenta, lung, liver, skeletal muscle, kidney, pancreas, spleen, thymus, prostate, testis, ovary, small intestine, colon, leukocyte, stomach, thyroid, spinal cord, lymph node, trachea, adrenal gland, bone marrow; BD Biosciences Clontech, Palo Alto, Calif.) were hybridized with a ³²P-labeled PCR product of CDCA5. The partial-length cDNA of CDCA5 was prepared by RT-PCR using primers CDCA5-F1 (GCTTGTAAAGTCCTCGGAAAGTT; SEQ ID NO: 27) and CDCA5-R1 (ATCTCAACTCTGCATCATCTGGT; SEQ ID NO: 28). Prehybridization, hybridization, and washing were performed according to the supplier's recommendations. The blots were autoradiographed with intensifying screens at −80 degrees C. for 7 days.

Anti-CDCA5 Antibodies.

Plasmids expressing full length CDCA5 that contained His-tagged epitopes at their N-terminals were prepared using pET28 vector (Novagen) and primers: CDCA5-F3 (5′-CCGGAATTCATGTCTGGGAGGCGAACGCG-3′; SEQ ID NO: 36) and CDCA5-R3 (5′-CCGCTCGAGTTCAACCAGGAGATCAAACTGCTC-3′; SEQ ID NO: 37). The recombinant proteins were expressed in Escherichia coli, BL21 codon-plus strain (Stratagene), and purified using Ni-NTA (QIAGEN) according to the supplier's protocol. The protein was inoculated into rabbits; the immune sera were purified on affinity columns according to standard methodology. Affinity-purified anti-CDCA5 antibodies were used for western blotting as well as immunocytochemical and immunohistochemical studies. It was confirmed that the antibody was specific to CDCA5 on western blots using lysates from cell lines that had been transfected with CDCA5 expression vector and those from lung cancer cell lines and airway epithelial cells, SAEC, either of which expressed CDCA5 endogenously or not.

Western-Blotting.

Cells were lysed in lysis buffer; 50 mM Tris-HCl (pH 8.0), 150 mM NaCl, 0.5% NP-40, 0.5% deoxycholate-Na, 0.1% SDS, plus protease inhibitor (Protease Inhibitor Cocktail Set III; Calbiochem). An ECL western-blotting analysis system (GE Healthcare Bio-sciences), as previously described (Kato T, Daigo Y, Hayama S, et al. Cancer Res 2005; 65:5638-46) was used.

Immunocytochemical Analysis.

Cultured cells were washed twice with PBS (−), fixed in 4% paraformaldehyde solution for 30 minutes at 37 degrees C., and then rendered permeable with PBS (−) containing 0.1% Triton X-100 for 3 minutes. Prior to the primary antibody reaction, cells were covered with blocking solution [3% bovine serum albumin in PBS (−)] for 10 minutes to block nonspecific antibody binding. After the cells were incubated with antibodies to human CDCA5 (generated to recombinant CDCA5; please see above), Alexa Fluor 488 goat anti-rabbit secondary antibody (Molecular Probes) was added to detect endogenous CDCA5. Nuclei were stained with 4′,6-diamidino-2-phenylindole (DAPI). The antibody-stained cells were viewed with a laser-confocal microscope (TSC SP2 AOBS: Leica Microsystems).

Immunohistochemistry and Tissue Microarray Analysis.

To investigate the significance of CDCA5 expression in clinical NSCLCs, tissue sections were stained using ENVISION+kit/horseradish peroxidase (HRP; DakoCytomation). Affinity-purified anti-CDCA5 antibody was added after blocking of endogenous peroxidase and proteins, and each section was incubated with HRP-labeled anti-rabbit IgG as the secondary antibody. Substrate-chromogen was added, and the specimens were counterstained with hematoxylin. Tumor tissue microarrays were constructed as published previously, using formalin-fixed NSCLCs (Callagy G, Cattaneo E, Daigo Y, et al. Diagn Mol Pathol 2003; 12:27-34, Callagy G, Pharoah P, Chin S F, et al. J Pathol 2005; 205:388-96, Chin S F, Daigo Y, Huang H E, et al. Molecular Pathology 2003; 56:275-9). Tissue areas for sampling were selected based on visual alignment with the corresponding H&E stained sections on slides. Three, four, or five tissue cores (diameter, 0.6 mm; height, 3-4 mm) taken from donor tumor blocks were placed into recipient paraffin blocks using a tissue microarrayer (Beecher Instruments). A core of normal tissue was punched from each case. Five-micrometer sections of the resulting microarray block were used for immunohistochemical analysis. Three independent investigators semi-quantitatively assessed CDCA5 positivity without prior knowledge of clinicopathological data. Since the intensity of staining within each tumor tissue core was mostly homogenous, the intensity of CDCA5 staining in the nucleus and cytoplasm was evaluated by recording as negative (no appreciable staining in tumor cells) or positive (brown staining appreciable in the nucleus and cytoplasm of tumor cells). Cases were accepted as positive only if all three reviewers independently defined them as such.

Statistical Analysis.

Statistical analyses were done using the StatView statistical program (SAS). It was used contingency tables to correlate clinicopathologic variables, such as age, gender, and pathologic tumor-node-metastasis (TNM) stage, with the positivity of CDCA5 protein determined by tissue-microarray analysis. Tumor-specific survival curves were calculated from the date of surgery to the time of death related to NSCLC, or to the last follow-up observation. Kaplan-Meier curves were calculated for each relevant variable and for CDCA5 expression; differences in survival times among patient subgroups were analyzed using the log-rank test. Univariate analysis was done with the Cox proportional-hazard regression model to determine associations between clinicopathologic variables and cancer-related mortality.

RNA Interference Assay.

Two independent CDCA5 siRNA oligonucleotides were designed using the CDCA5 sequences. The siRNAs (600 pM) were transfected into NSCLC cell lines LC319 and A549, using 30 micro-l of Lipofectamine 2000 (Invitrogen, Carlsbad, Calif.) following the manufacturer's protocol. The transfected cells were cultured for seven days. Cell numbers and viability were measured by Giemsa staining and triplicate 3-(4,5-dimethylthiazol-2-yl)-2,5-diphenyltetrazolium bromide (MTT) assay (cell counting kit-8 solution; Dojindo Laboratories). The siRNA sequences used were as follows: control-1 (si-LUC: luciferase gene from Photinus pyralis), 5′-NNCGUACGCGGAAUACUUCGA-3′ (SEQ ID NO: 29); control-2 (CNT: ON-TARGETplus siCONTROL Non-targeting siRNAs pool of 5′-UGGUUUACAUGUCGACUAA-3′ (SEQ ID NO: 30); 5′-UGGUUUACAUGUUUUCUGA-3′ (SEQ ID NO: 31); 5′-UGGUUUACAUGUUUUCCUA-3′ (SEQ ID NO: 32); 5′-UGGUUUACAUGUUGUGUGA-3′ (SEQ ID NO: 33)); siRNA-CDCA5-#1 (si-CDCA5-#1: 5′-GCAGUUUGAUCUCCUGGUUU-3′ (SEQ ID NO: 34)); siRNA-CDCA5-#2 (si-CDCA5-#2: 5′-GCCAGAGACUUGGAAAUGUUU-3′ (SEQ ID NO: 35)). Down-regulation of CDCA5 expression by functional siRNAs, but not by controls, was confirmed in the cell lines used for this assay.

Cell Growth Assay.

c-Myc/His-tagged CDCA5 expression vector (pcDNA3.1-c-Myc/His-CDCA5) or mock vector (pcDNA3.1-c-Myc/His) was transfected into COS-7 or NIH3T3 cells using FuGENE6 transfection reagent (Roche). Transfected cells were incubated in the culture medium containing 0.4 mg/ml, neomycin (Geneticin, Invitrogen). 7 days later, viability of cells was evaluated by MTT assay.

To establish COS-7 cells stably expressing CDCA5, non-tagged CDCA5 expression vector (pCAGGSn-CDCA5) or mock vector (pCAGGSn was transfected into COS-7 cells that weakly expressed endogenous CDCA5 using FuGENE6 transfection reagent (Roche). Transfected cells were incubated in the culture medium containing 0.4 mg/mL neomycin (Geneticin, Invitrogen) for 14 days. Then, 50 colonies were trypsinized and screened for stable transfectants by a limiting-dilution assay. Expression of CDCA5 was determined in each clone by RT-PCR, Western blotting and immunocytochemical staining. Viability of cells was evaluated by MTT assay at days 1, 3, 5, and 7.

In vitro Kinase Assay.

In vitro kinase assay was performed using full-length recombinant GST-CDCA5 (pGEX-6p-1/CDCA5 cleaved with Precision Protease). Briefly, 1.0 micro-g each of GST-CDCA5, Histone H1 (Upstate), MBP, or GST was incubated in 20 micro-l of kinase buffer (50 mM Tris-HCl, 10 mM MgCl2, 1 mM EGTA, 2 mM DTT, 0.01% Briji 35, 1 mMATP, pH7.5, 25 degrees C.) supplemented with 1 micro-Ci of [gamma-32P1-ATP (GE Healthcare) and 2 unit of CDC2 (BioLabs) or 50 ng of ERK2 (Upstate) for 20 min at 30 degrees C. The reactions were terminated with Laemmli SDS sample buffer to a final volume of 30 micro-l, and half of the samples were subjected to 5-15% gradient gel (Bio-Rad Laboratories), and phosphorylation were visualized by autoradiography. MBP was used as ERK substrate, and H1 as CDC2 substrate (positive control). GST was served as a negative control substrate.

MALDI-TOF Mass Spectrometry Analysis.

CDCA5 recombinant protein was incubated with ERK kinase (rhERK2) or CDC2 for 3.5 hours at 37 degrees C. Samples ware separated on SDS-PAGE gel. After electrophoresis, the gels were stained by R-250 (Bio-Rad). Specific bands corresponding to CDCA5 were digested with trypsin as previously described (Kato T et al. Clin Cancer Res 2008; 14: 2363-70) and served for analysis by matrix-assisted laser desorption/ionization mass spectrometry analysis (MALDI-QIT-TOF; Shimadzu Biotech, Kyoto, Japan). The mass spectral data was evaluated using the Mascot search engine (http://www.matrixscience.com) to identify proteins from primary sequence databases.

To determine in vivo ERK-dependent phosphorylation sites on CDCA5 in cultured cells, it was performed Colloidal CBB staining after immunoprecipitation of exogenously expressed CDCA5 protein in EGF-treated HeLa cells that were transfected with both CDCA5-expressing vectors and ERK2-expressing vectors, using anti-CDCA5 antibody. The bands corresponding to CDCA5 was excised for MS analysis as mentioned above.

EGF Stimulation Assay.

Cultured cervical squamous cell carcinoma HeLa cells were cultured in FCS free medium for 20 hours. Then, the cells were stimulated by 50 micro-g/ml EGF for 20 min with or without 10 micro-M MEK inhibitor U0126 (Promega).

Flow Cytometric Analysis.

The A549, LC319, and HeLa cells were synchronized their cell cycle by treatment with aphidicolin (Sigma-Aldrich) for 16 hours, washing three times with PBS (−), and adding fresh culture medium to release from the cell cycle arrest. For 14 hours (every 1 to 2 hours) after release from G1/S, cells were collected and fixed with 70% ethanol, and then kept at 4 degrees C. before use. The cells were incubated with 100 micro-g/ml RNase (Sigma-Aldrich) in PBS (−) at 37 degrees C. for 30 min and stained with 50 micro-g/ml propidium iodide (Sigma-Aldrich) at 4 degrees C. for 30 min. The cell suspensions at each time point were analyzed with FACScan (Becton Dickinson, Franklin Lakes, N.J.).

Results

Expression of CDCA5 in lung and esophageal cancers and normal tissues.

27,648 genes on a cDNA microarray were previously screened to detect transcripts indicating 3-fold or higher expression in cancer cells than in normal control cells in more than 40% of clinical lung and esophageal cancer samples analyzed. Among the up-regulated genes, the CDCA5 transcript was identified and its increased expression was confirmed in 9 of 10 representative NSCLC cases, all of 5 SCLC cases (FIG. 1A), and in all of the 23 lung-cancer cell lines by semiquantitative RT-PCR experiments. High levels of CDCA5 expression was also observed in all of the lung-cancer cell lines (FIG. 1B), whereas CDCA5 transcript was hardly detected in cells derived from normal small airway epithelia (SAEC) and normal esophagus samples. Furthermore, strong expression of endogenous CDCA5 protein was confirmed by western-blot analysis in lung cancer and esophageal cancer cell lines using anti-CDCA5 antibody (FIG. 1C). Immunofluorescence analysis was performed to examine the subcellular localization of endogenous CDCA5 in lung cancer LC319 cells and indicated that CDCA5 was located at the nucleus of interphase cells and weakly in cytoplasm (FIG. 1D). Northern blot analysis using a CDCA5 cDNA fragment as a probe identified a 2.8-kb transcript to be highly expressed in testis, but its transcript was hardly detectable in any other normal tissues (FIG. 2A). Furthermore, compared CDCA5 protein expressions in 5 normal tissues (heart, lung, liver, kidney, and testis) were compared with those in lung cancers by immunohistochemistry using anti-CDCA5 polyclonal antibodies. CDCA5 expression was detected abundantly in nucleus and weakly in cytoplasm of testis and lung cancer cells, but hardly detectable in the remaining four normal tissues (FIG. 2B).

Association of CDCA5 Expression with Poor Prognosis for NSCLC Patients.

Using tissue microarrays prepared from archived NSCLCs, it was performed immunohistochemical analysis with anti-CDCA5 polyclonal antibodies. The patterns of CDCA5 expression as negative or positive were classified (FIG. 2C). Of the 262 NSCLC cases examined, it was found positive staining in 192 cases (73.3%) and negative staining in 70 cases (26.7%) or in any of their adjacent non-cancerous cells (Table 1A). It was then examined the association of CDCA5 expression with various clinicopathological parameters of NSCLC patients who had undergone curative surgery, and found a significant association between CDCA5-positivity in NSCLCs and worse tumor-specific survival (P=0.0143 by log-rank test; FIG. 2D). It was also applied univariate analysis to evaluate associations between patient prognosis and several factors including age, gender, histological type (ADC versus non-ADC), pT stage (tumor size; T1 versus T2+T3), pN stage (node status; N0 versus N1+N2), and CDCA5 status (negative versus positive expression). All those parameters were significantly associated with poor prognosis (Table 1B). In multivariate analysis, CDCA5 status was indicated to be an independent prognostic factor for surgically treated lung cancer patients enrolled in this study (P=0.0237), while pT and pN stages as well as age did so.

[Table 1]

TABLE 1A
Association between CDCA5-positivity in NSCLC tissues
and patients' characteristics (n = 322)

| | | | CDCA5 | CDCA5 | | P- value |
|---|---|---|---|---|---|---|
| | | Total | positive | negative | | positive vs |
| | | n = 262 | n = 192 | n = 70 | χ² | negative |
| Age (years) | <65 | 128 | 98 | 30 | 1.375 | 0.2409 |
| | >=65 | 134 | 91 | 40 | | |
| Gender | Female | 88 | 64 | 24 | 0.021 | 0.8852 |
| | Male | 174 | 128 | 46 | | |
| Histological | ADC | 156 | 109 | 47 | 2.291 | 0.1301 |
| type | non-ADC | 106 | 83 | 23 | | |
| pT factor | T1 | 111 | 82 | 29 | 0.034 | 0.8528 |
| | T2 + T3 | 151 | 110 | 41 | | |
| pN factor | N0 | 204 | 150 | 54 | 0.029 | 0.8655 |
| | N1 + N2 | 58 | 42 | 16 | | |

ADC, adenocarcinoma
non-ADC, squamous-cell carcinoma plus large-cell carcinoma and adenosquamous-cell carcinoma
*P < 0.05 (χ² test)

TABLE 1B
Cox's proportional hazards model analysis
of prognostic factors in patients with NSCLCs

| | | | | |
|---|---|---|---|---|
| | Hazards | | Unfavorable/ | |
| Variables | ratio | 95% CI | Favorable | P-value |

Univariate analysis

| | | | | |
|---|---|---|---|---|
| CDCA5 | 2.266 | 1.156-4.442 | Positive/Negative | 0.0172* |
| Age (years) | 2.165 | 1.347-3.482 | >=65/65> | 0.0014* |
| Gender | 2.066 | 1.200-3.557 | Male/Female | 0.0089* |
| Histological type | 2.322 | 1.459-3.694 | non-ADC/ADC | 0.0004* |
| pT factor | 3.461 | 2.010-5.961 | T2 + T3/T1 | <0.0001* |
| pN factor | 4.456 | 2.807-7.072 | N1 + N2/N0 | <0.0001* |

Multivariate analysis

| | | | | |
|---|---|---|---|---|
| CDCA5 | 2.215 | 1.112-4.413 | Positive/Negative | 0.0237* |
| Age (years) | 1.852 | 1.103-3.112 | >=65/65> | 0.0198* |
| Gender | 1.285 | 0.675-2.444 | Male/Female | 0.4453 |
| Histological type | 1.516 | 0.843-2.726 | non-ADC/ADC | 0.1644 |
| pT factor | 2.373 | 1.234-4.565 | T2 + T3/T1 | 0.0096* |
| pN factor | 3.633 | 2.216-5.957 | N1 + N2/N0 | <0.0001* |

ADC, adenocarcinoma
non-ADC, squamous-cell carcinoma plus large-cell carcinoma and adenosquamous-cell carcinoma
*P < 0.05

Growth Promotive Activity of CDCA5.

The expression of endogenous CDCA5 was knocked down by siRNA in lung cancer cell lines A549 and LC319, which showed high levels of CDCA5 expression. The mRNA and protein expression levels of CDCA5 were examined, and it was found that two CDCA5-specific siRNAs (si-CDCA5-#1 and si-CDCA5-#2) significantly suppressed expression of CDCA5 mRNA and protein as compared with a control siRNA construct (si-LUC and si-CNT; FIG. 3A). Colony formation and MTT assays revealed that reduction of CDCA5 expression by the two si-CDCA5s significantly suppressed the growth of both A549 and LC319 cells (FIGS. 3B and 3C), in accordance with its knockdown effect on CDCA5 expression. Next, a possible role of CDCA5 was examined in cell growth-promoting effect. Plasmids designed to express non-tagged full-length CDCA5 (pcDNA3.1-CDCA5) or mock plasmids were prepared and transfected into COS-7 or NIH3T3 cells, and established two independent COS-7 cell lines overexpressing exogenous CDCA5 (COS-7-CDCA5-#A and -#B) and two control cells (COS-7-Mock-#A and -#B). It was then carried out MTT assay of these COS-7-derived transfectants and compared the growth of COS-7-CDCA5 cells with control COS-7-Mock cells. Transfection of CDCA5 cDNA into COS-7 or NIH3T3 cells significantly enhanced cell growth, compared with that of mock vector. Growth of the two COS-7-CDCA5 cells was promoted at a significant degree in accordance with the expression level of CDCA5 as detected by western-blot analysis (FIG. 3D).

Phosphorylation of CDCA5 by ERK and CDC2 Protein Kinases in vitro.

To analyze the function of CDCA5 in carcinogenesis, the possible phosphorylation of CDCA5 protein were focused, because in silico approach suggested several consensus phosphorylation sites on CDCA5 protein by ERK kinase [x-x-S/T-P] that is one of important downstream components of oncogenic MAPK pathway. According to the previous report using proteomic phospho-peptides screening, CDCA5 was supposed to be phosphorylated at Serine-75, Serine-79, and Threonine-115 (Olsen Jv et al. Cell 2006; 127(3):635-648). To identify the cognate kinase for CDCA5 phosphorylation, the peptide sequence of CDCA5 including Serine-75, Serine-79, and Threonine-115 with possible phosphorylation sites were compared, and it was found that Serine-75 of CDCA5 completely matched the consensus CDC2 protein kinase phosphorylation site [S/T-P-x-R/K], while Serine-79 and Threonine-115 concordantly matched the ERK phosphorylation site [x-x-S/T-P]. These consensus sequences were highly conserved in many species. In vitro kinase assay were subsequently performed by incubating recombinant rhERK2 with rhCDCA5, and indicated that CDCA5 was directly phosphorylated by both ERK kinase (FIG. 4A). The results suggested that CDCA5 could be involved in the ERK pathway.

To determine the direct phosphorylation sites on CDCA5 by ERK, in vitro kinase assay coupled with subsequent Mass spectrometric analysis. After the kinase assay and following separation of proteins by SDS-PAGE, three bands corresponding to rhCDCA5 proteins that were incubated with or without rhERK2 were digested with trypsin and served for MALDI-QIT-TOF MS analysis was performed (FIG. 4B).

Identification of ERK-Dependent in vivo Phosphorylation of CDCA5.

To prove that endogenous CDCA5 was phosphorylated by ERK in mammalian cells, serum-starved HeLa cells were stimulated with EGF in the presence or absence of MEK inhibitor U0126. Western blotting using anti-ERK antibody detected upper shifted bands indicated that ERK was highly activated at 15 and 30 minutes after EGF stimulation, but the level was decreased at 60 minute (FIG. 5A, left panel). In accordance with the increased levels of ERK phosphorylation, a CDCA5 band detected by anti-CDCA5 antibody shifted to higher molecular weights. In contrast, treatment of the cells with both EGF and MEK inhibitor U0126 reduced the levels of ERK phosphorylation and reduced the levels of ERK phosphorylation and completely inhibited the upper shift of CDCA5 band (FIG. 5A, right panels). These results demonstrate the possible phosphorylation of endogenous CDCA5 protein by ERK pathway.

To confirm MAP kinase pathway-dependent phosphorylation of CDCA5 and identify the phosphorylation sites in cultured cells, HeLa cells transfected with plasmids designed to express myc-tagged CDCA5 were stimulated with EGF in the presence or absence of MEK inhibitor U0126, and their cell extracts were served for 2D-Western-blotting using anti-myc antibody. In HeLa cells without treatment of EGF and U0126, 2 spots were detected (spots no. 1 and 2), however, treatment with EGF resulted in relatively remarkable increase in the signal of one of the spots (spot no. 2), while it induced two new spot signals (spots no. 3 and 4) with more acidic pI values. These shifted spots with more acidic pI were significantly reduced by pre-incubation of the cells with MEK inhibitor U0126. In addition, the signal of spot no. 2 that had been increased by EGF stimulation was also reduced by U0126 treatment. These results suggest that CDCA5 was specifically phosphorylated by MAPK cascade in response to EGF ligand stimulation.

Identification of ERK-Dependent in vivo Phosphorylation of CDCA5.

According to in vitro kinase assay using ERK kinase and CDCA5 as a substrate, phosphorylated CDCA5 was detected as shifted band. In addition, endogenous CDCA5 was detected as a shifted band in HeLa cells under the EGF stimulation condition (FIG. 5A). To confirm the phosphorylation status of none-tagged CDCA5 in cultured cells, HeLa cells transfected with exogenous CDCA5 expression vector was stimulated with EGF. The shifted band of ERK2 indicated the activation of endogenous ERK in cells. But, no phosphorylated CDCA5 protein was detected as shifted bands (FIG. 5B). To identify ERK-dependent phosphorylation sites in cultures cells, immunoprecipitation assay was performed using anti-CDCA5 antibody to immunoprecipitate non-tagged CDCA5 protein from EGF-non-treated cell lysates. Only Ser21 was confirmed in all samples. These results indicated that endogenous ERK in cancer cells might not be enough to stimulated overexpressed exogenous CDCA5 protein. Subsequently, exogenous ERK1 or 2 were transfected to HeLa cells. After stimulation of the cells with EGF, the activation of exogenous ERK1 or 2 as well as endogenous ERK were detected in EGF stimulation cells, but not in U0126 MEK inhibitor treatment. In accordant with ERK activation, the phosphorylated exogenous CDCA5 could be detected as shifted bands (FIG. 6A). These results indicated that CDCA5 was phosphorylated by overexpressed ERK in HeLa cells. To determine ERK-dependent phosphorylation sites on CDCA5, Colloidal CBB staining was performed after immunoprecipitation of this non-tagged CDCA5 protein using anti-CDCA5 antibody. The cells were prepared under conditions with EGF-non-treated or EGF stimulated with or without MEK inhibitor, U0126. The bands corresponding to CDCA5 were excised for MS analysis using 4800 plus MALDI-TOF-TOF analyzer (FIG. 6B). The MS analysis showed that 70%, 77% and 66% of CDCA5 sequence were covered in non-treated, EGF stimulated, and EGF stimulated with MEK inhibitor, respectively. Two ERK-dependent phosphorylation sites Ser 79 and Ser209 were identified. Phospho-Ser21 was found in all samples, indicating that it is not an ERK-dependent phosphorylation site (FIGS. 6C-6E). To confirm the results of the MS analysis, Western-blot analysis was performed using cells transfected with non-tagged CDCA5 vector whose Ser79 and/or Ser209 residues were replaced with alanine. ERK2 expressing vector was co-transfected with wild type and mutant CDCA5 expressing vectors under non-treated or EGF stimulation conditions. As shown in the Western blotting, the shifted band of ERK2 showed the activation of ERK by EGF stimulation. Wild type CDCA5 was detected as shifted bands after EGF stimulation. The levels of upper-shifted bands for CDCA5-S79A and CDCA5-S209A were weaker compared to wild type CDCA5, while no shifted-band of double mutant CDCA5 was detected (FIG. 6F). These results indicated that Ser79 and Ser209 might be phosphorylated by ERK in cells.

To date, there are many evidences reporting that MARK pathway promotes carcinogenesis. Furthermore, these data suggested that CDCA5 was characterized as cancer testis antigen, one of the causatives in lung or esophageal carcinogenesis. To examine whether the function of ERK-dependent phosphorylation sites on CDCA5 might play important roles in cancer progression, the dominant negative effect of these phosphorylation sites on cancer cell growth were investigated. Growth assay was performed using CDCA5-Ser79 or Ser209 alanine substitutes. The MTT assay showed that transfection of Ser209 alanine substitute inhibited the growth of lung cancer cell line A549 and LC319 lung cancer cell lines. This might suggest that Ser209 alanine substitute showed dominant negative effect on these cell lines (FIG. 7A). To confirm the effect of these phosphorylation sites on cell growth, Ser79 or Ser209 phospho-mimicking constructs whose Ser residues were replaced with aspargine acid or glutamine acid were expressed in LC319 lung cancer cell line. The MTT assay showed that only cells transfected with the Ser209 phospho-mimicking vector could significantly promote growth of LC319 and A549 cells (FIG. 7B). The results could strongly suggest that phosphorylation of Ser209 on CDCA5 plays an important role for cancer cell growth.

Discussion

Molecular-targeted drugs are expected to be highly specific to malignant cells, and to have minimal adverse effects due to their well-defined mechanisms of action. In spite of improvement of model surgical techniques and adjuvant chemo-radiotherapy, lung cancer and ESCC are known to reveal the worst prognosis among malignant tumors. Therefore, it is now urgently required to develop novel diagnostic biomarkers for the early detection of these cancers and for the better choice of adjuvant treatment modalities to individual patients, as well as new types of anti-cancer drugs and/or cancer vaccines. To identify appropriate diagnostic and therapeutic target molecules, genome-wide expression analysis was combined (Kikuchi T et al. Oncogene 2003; 22:2192-2205, Kakiuchi S et al. Mol Cancer Res 2003; 1:485-499, Kakiuchi S et al. Hum Mol Genet 2004; 13:3029-3043, Kikuchi T et al. Int J Oncol 2006; 28:799-805, Taniwaki M et al. Int J Oncol 2006; 29:567-75, Yamabuki T et al. Int J Oncol 2006; 28:1375-84) for selecting genes that were overexpressed in lung and esophageal cancer cells by high-throughput screening of loss-of-function effects by means of the RNAi technique (Suzuki C et al. Cancer Res 2003; 63:7038-7041, Ishikawa N et al. Clin Cancer Res 2004; 10:8363-8370, Kato T et al. Cancer Res. 2005; 65:5638-46, Furukawa C et al. Cancer Res. 2005; 65(16):7102-10, Ishikawa N et al. Cancer Res. 2005; 65(20):9176-84, Suzuki C et al. Cancer Res. 2005; 65:11314-25, Ishikawa Net al. Cancer Sci 2006; 97:737-45, Takahashi K et al. Cancer Res 2006; 66:9408-19, Hayama S et al. Cancer Res 2006; 66:10339-48, Kato T et al. Clin Cancer Res 2007; 13:434-42, Suzuki C et al. Mol Cancer Ther 2007; 6:542-551, Yamabuki T et al. Cancer Res 2007; 67:2517-25, Hayama S et al. Cancer Res 2007; 67:4113-22, Kato T et al. Cancer Res 2007; 67:8544-53, Taniwaki M et al. Clin Cancer Res 2007; 13:6624-31, Ishikawa N, et al. Cancer Res 2007; 67:11601-11, Mano Yet al. Cancer Sci 2007; 98:1902-13, Suda T et al. Cancer Sci 2007; 98: 1803-8, Mizukami Y et al. Cancer Sci 2008; 99:1448-54). Using this systematic approach, it was found CDCA5 to be frequently overexpressed in clinical lung cancer and ESCC samples, and showed that overexpression of this gene product plays an indispensable role in the growth of lung-cancer cells.

Previous studies have demonstrated that CDCA5 interacts with cohesion on chromatin and functions there during interphase to support sister chromatid cohesion, and sister chromatids are further separated than normally in most G2 cells, suggesting the possibility that CDCA5 is already required for establishment of cohesion during S phase (Schmitz J et al. Curr Biol 2007; 17: 630-636). So far only one other protein is known to be specifically required for cohesion establishment: the budding yeast acetyl-transferase Eco1/Ctf7 (Skibbens R V et al. Genes Dev 1999; 13:307-319, Toth A et al. Genes Dev 1999; 13:320-333, Ivanov D et al. Curr. Biol 2002; 12:323-328). Homologs of this enzyme are also required for cohesion in Drosophila and human cells (Williams B C et al. Curr. Biol 2003; 13: 2025-2036, Hou F and Zou H. Mol Biol Cell 2005; 16:3908-3918), although it is not yet known whether these proteins also function in S phase. It will therefore be interesting to address whether CDCA5 and Eco1/Ctf7 homologs collaborate to establish cohesion in cancer cells.

Sister chromatid cohesion must be established and dismantled at the appropriate times in the cell cycle to effectively ensure accurate chromosome segregation. It has previously been shown that the activation of APCCdc20 controls the dissolution of cohesion by targeting the anaphase inhibitor securin for degradation. This allows the separase-dependent cleavage of Scc1/Rad21, triggering anaphase. The degradation of most cell cycle substrates of the APC is logical in terms of their function; degradation prevents the untimely presence of activity and in a ratchet-like way promotes cell cycle progression. The function of CDCA5 may also be redundant with that of other factors that regulate cohesion, with their combined activities ensuring the fidelity of chromosome replication and segregation (Rankin S et al. Mol. Cell 2005; 18:185-200). According to our microarray data, APC, and CDC20 were also highly expressed in lung and esophageal cancers; although their expressions in normal tissues are low. Furthermore, high expression of CDC20 was also confirmed in clinical small cell lung cancer using semi-quantitative RT-PCR and immunohistochemical analysis (Taniwaki M et al. Int J Oncol 2006; 29:567-75). These data implied that CDCA5 in collaboration with CDC20 might enhance the growth of cancer cells, by promoting cell cycle progression, although, no evidence shows that these molecules could interact directly with CDCA5.

CDCA5 was previously reported to be located in the nucleus at interphase, cytosolic in Mitosis (Rankin S et al. Mol. Cell 2005; 18:185-200). However, its physiological function remains unclear. It was confirmed that CDCA5 localized at the nucleus. The nucleus contains genetic materials and its main function is to maintain the integrity of the genes and regulate gene expression. The nucleus is a dynamic structure that changes according to the cells requirements. In order to control the nuclear functions, the processes of entry and exit from the nucleus are regulated. The localization of CDCA5 in the nucleus indicates that this molecule may play roles as an essential factor to control cell cycle (Kho C J, et al. Cell Growth Differ 1996; 7:1157-1166, Bader N, et al. Exp Gerontol 2007 [Epub ahead of print]). Although, CDCA5 was known to play important roles in cell cycle control through its interaction with cohesion on chromatin (Schmitz J, Watrin E, Lenart P, et al. Curr Biol 2007; 17:630-636), no studies proved that CDCA5 has any relationship with carcinogenesis process. It was confirmed that CDCA5 is a putative oncogene that is aberrantly expressed in lung cancer cells. It was found by tissue microarray analysis that patients with NSCLC showing higher expression of CDCA5 protein represented a shorter tumor-specific survival period, strongly indicating that CDCA5 plays a crucial role for progression of lung cancers.

Our data also suggested that the CDCA5 was likely to be phosphorylated by ERK at two phosphorylation sites serine-79 and serine-209, whose sequences as a consensus ERK phosphorylation site were highly conserved in many species (data not shown) and that phosphorylation of serine-209 by ERK seemed to be important for cell growth. The ERK is a member of the MAP kinase family proteins that function as an integration point for multiple biochemical signals, and are involved in a wide variety of cellular processes such as proliferation, differentiation, transcription regulation, and development (Roux P P and Blenis J. Microbiol. Mol Biol Rev 2004; 68:320-44, Chang L and Karin M. Nature 2001; 410:37-40, Ferrell J E Jr. Trends Biochem Sci 1996; 21:460-6). Aberrant regulation of MAPK cascades was well known to contribute to carcinogenesis (Cowley S, Paterson H, Kemp P, Marshall C J. Cell 1994; 77:841-52, Mansour S J, Matten W T, Hermann A S, et al. Science 1994; 265:966-70). The Raf-MEK-ERK pathway is a key downstream effecter of the Ras small GTPase, which is a key downstream effecter of the EGFR (Roux P P and Blenis J. Microbiol. Mol Biol Rev 2004; 68:320-44, Chang L and Karin M. Nature 2001; 410:37-40, Ferrell J E Jr. Trends Biochem Sci 1996; 21:460-6). Thus, the EGFR-Ras-Raf-MEK-ERK signaling network has been the subject of intense research scrutiny leading to the discovery of new anti-cancer drugs. Currently, inhibitors of the Raf-MEK-ERK cascade like geldanamycin analog 17-allylamino-17-demethoxygeldanamycin (17-AAG) are under evaluation in clinical trials (Smith R A, Dumas J, Adnane L, Wilhelm S M. Curr Top Med Chem 2006; 6:1071-89). Furthermore, small molecule MEK inhibitors, Ras inhibitors were being developed (English J M and Cobb M H. Trends Pharmacol Sci 2002; 23:40-5). Although these drugs had proven to be effective in preclinical studies and/or in certain proportion of cancer patients, clinical response was not likely to be precisely correlated with the activation level of target proteins. In addition, severe adverse reactions due to their non-specific cytotoxicity were reported in some cases. Therefore, more specific targeting of this pathway based on the clarification of unknown downstream oncogenic signals may be one of the promising approaches.

It was demonstrated that growth of lung-cancer cells over-expressing CDCA5 could be suppressed effectively by dominant negative effect by mutant CDCA5 protein whose ERK-dependent phosphorylated residue of serine-209 was replaced with alanine. Since the phosphorylation of CDCA5 at the site was likely to be indispensable for the growth/survival of lung cancer cells, and CDCA5 could belong to cancer-testis antigens, selective targeting of CDCA5-ERK enzymatic activity as well as cancer immunotherapy such as cancer vaccine could be a promising therapeutic strategy that is expected to have a powerful biological activity against cancer with a minimal risk of adverse events.

In summary, CDCA5 is likely to play a significant role in lung carcinogenesis through its phosphorylation at serine-209 by MAPK pathway. Inhibition of CDCA5 expression as well as its functional interaction with ERK kinase could be a promising therapeutic strategy for the development of new type of anti-cancer drugs.

### INDUSTRIAL APPLICABILITY

As demonstrated herein, ERK has kinase activity for CDCA5, and the suppression of this activity leads to the inhibition of cell proliferation of cancer cells. Thus, agents that inhibit the kinase activity of ERK for CDCA5 find therapeutic utility as anti-cancer agents for the treatment of lung cancer or esophageal cancer. The phosphorylation site of CDCA5 by ERK is, for example, Ser79 or Ser209.

In addition, the present invention provides a screening method for anti-cancer agents that inhibit the kinase activity of ERK for CDCA5. Accordingly, it is expected that candidate compounds that inhibit the critical step for cell proliferation can be isolated by the present invention.

In addition, the present invention demonstrates that treatment of cancer cells with CDCA5 mutant, such as CDCA5 (S209A), suppresses the kinase activity of ERK for CDCA5 at Ser209, and, thus, suppresses growth of cancer cells. This data implies that up-regulation of ERK function and enhancement of the kinase activity of ERK for CDCA5 are common features of pulmonary carcinogenesis. Accordingly, the selective suppression of ERK kinase activity may be a promising therapeutic strategy for the treatment of lung and esophageal cancer patients.

All patents, patent applications, and publications cited herein are incorporated by reference in their entirety. However, nothing herein should be construed as an admission that the invention is not entitled to antedate such disclosure by virtue of prior invention.

While the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Further advantages and features will become apparent from the claims filed hereafter, with the scope of such claims to be determined by their reasonable equivalents, as would be understood by those skilled in the art. Thus, the invention is intended to be defined not by the above description, but by the following claims and their equivalents.

1.-6. (canceled)

7. A substantially pure polypeptide selected from the group consisting of
a. a polypeptide comprising the amino acid sequence of SEQ ID NO: 7;
b. a polypeptide that comprises the amino acid sequence of SEQ ID NO: 7 in which one or more amino acids are substituted, deleted, inserted, and/or added and that has a biological activity equivalent to the polypeptide consisting of the amino acid sequence of SEQ ID NO: 7; and
c. a polypeptide encoded by a polynucleotide that hybridizes under stringent conditions to a polynucleotide consisting of the nucleotide sequence of SEQ ID NO: 6, wherein the polypeptide has a biological activity equivalent to the polypeptide consisting of the amino acid sequence of SEQ ID NO: 7.

8. An isolated polynucleotide encoding the polypeptide of claim

9. A vector comprising the polynucleotide of claim 8.

10. A host cell harboring the polynucleotide of claim 8.

11. A method for either or both treating and preventing lung or esophageal cancer in a subject, said method comprising the step of administering a CDCA5 polypeptide mutant having a dominant negative effect, a polynucleotide encoding said mutant, or a vector comprising the polynucleotide.

12. The method of claim 11, wherein the CDCA5 polypeptide mutant comprises an amino acid sequence in which at least one ERK-dependent phosphorylation site on CDCA5 polypeptide is substituted with an amino acid residue other than that of the wild type.

13. The method of claim 12, wherein the ERK-dependent phosphorylation site is either or both Ser-79, and Ser-209.

14. The method of claim 13, wherein the CDCA5 polypeptide mutant comprises the amino acid sequence of SEQ ID NO: 7.

15. The method of claim 14, wherein the CDCA5 polypeptide mutant has the general formula:
[R]-[D],
wherein [R] is a membrane transducing agent, and [D] is a polypeptide comprising the amino acid sequence of SEQ ID NO: 7.

16. The method of claim 15, wherein the membrane transducing agent is selected from group consisting of;
poly-arginine;
SEQ ID NO: 8
Tat/RKKRRQRRR/;
SEQ ID NO: 9
Penetratin/RQIKIWFQNRRMKWKK/;
SEQ ID NO: 10
Buforin II/TRSSRAGLQFPVGRVHRLLRK/;
SEQ ID NO: 11
Transportan/GWTLNSAGYLLGKINLKALAALAKKIL/;
SEQ ID NO: 12
MAP (model amphipathic peptide)/
KLALKLALKALKAALKLA/;
SEQ ID NO: 13
K-FGF/AAVALLPAVLLALLAP/;
SEQ ID NO: 14
Ku70/VPMLK/;
SEQ ID NO: 15
Ku70/PMLKE/;
SEQ ID NO: 16
Prion/MANLGYWLLALFVTMWTDVGLCKKRPKP/;
SEQ ID NO: 17
pVEC/LLIILRRRIRKQAHAHSK/;
SEQ ID NO: 18
Pep-1/KETWWETWWTEWSQPKKKRKV/;
SEQ ID NO: 19
SynB1/RGGRLSYSRRRFSTSTGR/;
SEQ ID NO: 20
Pep-7/SDLWEMMMVSLACQY/;
and
SEQ ID NO: 21
HN-1/TSPLNIHNGQKL/.

17. A composition for either or both treating and preventing lung or esophageal cancer, said composition comprising a pharmaceutically effective amount of a CDCA5 polypeptide mutant having a dominant negative effect, a polynucleotide encoding said mutant, or a vector comprising the polynucleotide as an active ingredient, and a pharmaceutically acceptable carrier.

18.-25. (canceled)

26. A host cell harboring the vector of claim 9.
